SEQUENCE LISTING (28 sequences)

SEQ ID NO: 1 - DNA, 5899 bp, Synthetic construct
  source 1..5899: mol_type = other DNA
agctttgcagccgggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtaattgaaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtaaccgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagggctgcagcatgcctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtatcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgatatcaagcttggtaccgagctcggatccactagtaacggccgccagtgtgctggaattcgcccttccgcagcgccagcatgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatctcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgagacatcagggtggcaagccgaattccccctagcggaagggcgaattctgcagaatcggatccatcgataccgtcgacctcgagggggggcccggtacccaattcgccctatagtgagtcgtattacgcgcgcagcggccgaccatggcccaacttgtttattgcagcttataatggttacaaataaagcaatagcatcacaaatttcacaaataaagcatttttttcactgcattctagttgtggtttgtccaaactcatcaatgtatcttatcatgtctggatctccggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggtctgagacaataaccctgataaatgcttcaataatgta

SEQ ID NO: 2 - DNA, 7096 bp, Synthetic construct
  source 1..7096: mol_type = other DNA
agctttgcagccgggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtaattgaaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtaaccgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagggctgcagcatgcctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctggtggggtagggatgagggagggaggggcattgtgatgtacagggctgctctgtgagatcaagggtctcttaagggtgggagctggggcagggactacgagagcagccagatgggctgaaagtggaactcaaggggtttctggcacctacctacctgcttcccgctggggggtggggagttggcccagagtcttaagattggggcagggtggagaggtgggctcttcctgcttcccactcatcttatagctttctttccccagatccgaattcgagatccaaaccaaggaggaaaggatatcacagaggagacctcgagcccatcaacaagtttgtacaaaaaagcaggcaccatgctgccgtgcctcgtagtgctgctggcggcgctcctcagcctccgtcttggctcagacgctcatgggacagagctgcccagccctccgtctgtgtggtttgaagcagaatttttccaccacatcctccactggacacccatcccaaatcagtctgaaagtacctgctatgaagtggcgctcctgaggtatggaatagagtcctggaactccatctccaactgtagccagaccctgtcctatgaccttaccgcagtgaccttggacctgtaccacagcaatggctaccgggccagagtgcgggctgtggacggcagccggcactccaactggaccgtcaccaacacccgcttctctgtggatgaagtgactctgacagttggcagtgtgaacctagagatccacaatggcttcatcctcgggaagattcagctacccaggcccaagatggcccccgcaaatgacacatatgaaagcatcttcagtcacttccgagagtatgagattgccattcgcaaggtgccgggaaacttcacgttcacacacaagaaagtaaaacatgaaaacttcagcctcctaacctctggagaagtgggagagttctgtgtccaggtgaaaccatctgtcgcttcccgaagtaacaaggggatgtggtctaaagaggagtgcatctccctcaccaggcagtatttcaccgtgaccaacgtcatcatcttctttgcctttgtcctgctgctctccggagccctcgcctactgcctggccctccagctgtatgtgcggcgccgaaagaagctacccagtgtcctgctcttcaagaagcccagccccttcatcttcatcagccagcgtccctccccagagacccaagacaccatccacccgcttgatgaggaggcctttttgaaggtgtccccagagctgaagaacttggacctgcacggcagcacagacagtggctttggcagcaccaagccatccctgcagactgaagagccccagttcctcctccctgaccctcacccccaggctgacagaacgctgggaaacggggagccccctgtgctgggggacagctgcagtagtggcagcagcaatagcacagacagcgggatctgcctgcaggagcccagcctgagccccagcacagggcccacctgggagcaacaggtggggagcaacagcaggggccaggatgacagtggcattgacttagttcaaaactctgagggccgggctggggacacacagggtggctcggccttgggccaccacagtcccccggagcctgaggtgcctggggaagaagacccagctgctgtggcattccagggttacctgaggcagaccagatgtgctgaagagaaggcaaccaagacaggctgcctggaggaagaatcgcccttgacagatggccttggccccaaattcgggagatgcctggttgatgaggcaggcttgcatccaccagccctggccaagggctatttgaaacaggatcctctagaaatgactctggcttcctcaggggccccaacgggacagtggaaccagcccactgaggaatggtcactcctggccttgagcagctgcagtgacctgggaatatctgactggagctttgcccatgaccttgcccctctaggctgtgtggcagccccaggtggtctcctgggcagctttaactcagacctggtcaccctgcccctcatctctagcctgcagtcaagtgagcgggccaagcggcagctgctgaacttcgacctgctgaagctggccggcgacgtggagagcaaccccgggcccatgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatctcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgaacccagctttcttgtacaaagtggtgctagcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtaccggttagtaatgatcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggcgatggtacccaattcgccctatagtgagtcgtattacgcgcgcagcggccgaccatggcccaacttgtttattgcagcttataatggttacaaataaagcaatagcatcacaaatttcacaaataaagcatttttttcactgcattctagttgtggtttgtccaaactcatcaatgtatcttatcatgtctggatctccggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggtctgagacaataaccctgataaatgcttcaataatgta

SEQ ID NO: 3 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 4 - protein, 178 aa, Rattus norvegicus
MPGSALLCCLLLLAGVKTSKGHSIRGDNNCTHFPVSQTHMLRELRAAFSQVKTFFQKKDQLDNILLTDSLLQDFKGYLGCQALSEMIKFYLVEVMPQAENHGPEIKEHLNSLGEKLKTLWIQLRRCHRFLPCENKSKAVEQVKNDFNKLQDKGVYKAMNEFDIFINCIEAYVTLKMKN

SEQ ID NO: 5 - protein, 178 aa, Rattus norvegicus
MLGSALLCCLLLLAGVKTSKGHSIRGDNNCTHFPVSQTHMLRELRAAFSQVKTFFQKKDQLDNIVLTDSLLQDFKGYLGCQALSEMIKFYLVEVMPQAENHGPEIKEHLNSLGEKLKTLWIQLRRCHRFLPCENKSKAVEQVKNDFNKLQDKGVYKAMNEFDIFINCIEAYVTLKMKN

SEQ ID NO: 6 - protein, 178 aa, Mus musculus
MPGSALLCCLLLLTGMRISRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 7 - protein, 178 aa, Equus caballus
MHSSALLCYLVFLAGVGASRDRGTQSENSCTHFPTSLPHMLHELRAAFSRVKTFFQMKDQLDNMLLNGSLLEDFKGYLGCQALSEMIQFYLEEVMPQAENHGPDIKEHVNSLGEKLKTLRVRLRRCHRFLPCENKSKAVEQVKSAFSKLQEKGVYKAMSEFDIFINYIEAYMTTKMKN

SEQ ID NO: 8 - protein, 178 aa, Felis catus
MHSSALLCFLVFLAGVGASRHQSTLSEDNCTHFSVSLPHMLRELRAAFGKVKTFFQTKDELHSILLTRSLLEDFKGYLGCQALSEMIQFYLEEVMPQAENEDPDIKQHVNSLGEKLKTLRLRLRRCHRFLPCENKSKVVEQVKSTFSKLQEKGVYKAMGEFDIFINYIEAYMTMKMKI

SEQ ID NO: 9 - protein, 178 aa, Cavia porcellus
MPGSALLCCLALLAGVKASQGTNTQSEDSCAHFPAGLPHMLRELRAAFGRVKTFFQTQDQLDNVLLNKSLLEDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 10 - protein, 178 aa, Lama glama
MPRSALLCCLILLAGVAASRDQGTQSENSCAHFPASLPHMLRELRAAFGRVKTFFQMKDQLDNMLLTRSLLEDFKGYLGCQALSEMIQFYLEEVMPQAENHGPDIKEHVNSLGEKLKTLRLRLRRCHRFLPCENKSKAVEQVRGVFSKLQEKGVYKAMSEFDIFINYIEAYMTMKMKN

SEQ ID NO: 11 - protein, 178 aa, Meriones unguiculatus
MPSSALLYCLILLAGVRPSRGEYPRNESNCTHFPVSQTHMLRELRAAFSQVKTFFQKKDQLDNILLTDSLQQDFKGYLGCQALSEMIQFYLVEVMPQAENHGPEIKENLNSLGEKLKTLRMQLRRCHRFLPCENKSKAVEQVKNDFNKLQEKGVYKAMNEFDIFINCIEAYMTIKMKS

SEQ ID NO: 12 - protein, 178 aa, Pan troglodytes
MHSSALLCCLVLLTGVRASPGQGTQSENSCTHFPGNLPNMLRDLRDAFSRVKTFFQMKDQLDNLLLKESLLEDFKGYLGCQALXEMIQFYLEEVMPQAENQDPDIKVHVNSLGENLKTLRLRLRRCHRFLPCENKSKAVEQVKNAFNKLQEKGIYKAMSEFDIFINYIEAYMTMKIRN

SEQ ID NO: 13 - protein, 178 aa, Macaca mulatta
MHSSALLCCLVLLTGVRASPGQGTQSENSCTRFPGNLPHMLRDLRDAFSRVKTFFQMKDQLDNILLKESLLEDFKGYLGCQALSEMIQFYLEEVMPQAENHDPDIKEHVNSLGENLKTLRLRLRRCHRFLPCENKSKAVEQVKNAFSKLQEKGVYKAMSEFDIFINYIEAYMTMKIQN

SEQ ID NO: 14 - protein, 178 aa, Callithrix jacchus
MHSSALLCCLVFLTGVRASPGQGTQSENSCTHFPGSLPHMLRELRVAFSRVKTFFQKKDQLDSMLLKESLLEDFKGYLGCQALSEMIQFYLEEVMPQAENHDPDIKEHVNSLGEKLKTFRLRLRRCHRFLPCENKSKAVVQVKNAVSKLQEKGIYKAMSEFDIFIDYIEAYMTMKAQN

SEQ ID NO: 15 - protein, 178 aa, Macaca nemestrina
MHSSALLCCLVLLTGVRASPGQGTQSENSCTRFPGNLPHMLRDLRDAFSRVKTFFQMKDQLDNILLKESLLEDFKGYLGCQALSEMIQFYLEEVMPQAENHDPDIKEHVNSLGENLKTLRLRLRRCHRFLPCENKSKAVEQVMNAFSKLQEKGVYKAMSEFDIFINYIEAYMTMKIQN

SEQ ID NO: 16 - protein, 178 aa, Macaca fascicularis
MHSSALLCCLVLLTGVRASPGQGTQSENSCTRFPGNLPHMLRDLRDAFSRVKTFFQMKDQLDNILLKESLLEDFKGYLGCQALSEMIQFYLEEVMPQAENHDPDIKEHVNSLGENLKTLRLRLRRCHRFLPCENKSKAVEQVKNAFSKLQEKGVYKAMSEFDIFINYIEAYMTMKIRN

SEQ ID NO: 17 - protein, 118 aa, Cercocebus atys
MHSSALLCCLVLLTGVRASPGQGTQSENSCTRFPGNLPHMLRDLRDVFSRVKTFFQMKDQLDNILLKESLLEDFKGYLGCQALSEMIQFYLEEVMPQAENHDPDIKEHVNSLGENLKT

SEQ ID NO: 18 - protein, 178 aa, Saimiri sciureus
MHSSALLCCLVFLTGVRASPGQGTQSENSCTHFPGSLPHMLRELRVAFGRVKTFFQKKDQLDSMLLKESLLEDFKGYLGCQALSEMIQFYLEEVMPQAENHDPDIKEHVNSLGEKLKTFRLRLRRCHRFLPCENKSKAVAQVKNAVSKLQEKGVYKAMSEFDIFIDYIEAYMTMKTQN

SEQ ID NO: 19 - protein, 177 aa, Capra hircus
MPSSSALLCCLVFLAGVAASRDASTLSDSSCTHFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 20 - protein, 177 aa, Ovis aries
MPSSSAVLCCLVFLAGVAASRDASTLSDSSCTHFPASLPHMLRDVRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 21 - protein, 181 aa, unidentified
  source 1..181: note = Canis familiaris
MHGSALLCCCLVLLAGVGASRHQSTLLEDDCTHFPASLPHMLRELRAAFGRVKIFFQMKDKLDNILLTGSLLEDFKSYLGCQALSEMIQFYLEEVMPRAENHDPDIKNHVNSLGEKLKTLRLRLRLRRCHRFLPCENKSKAVEQVKSAFSKLQEKGVYKAMSEFDIFINYIETYMTMRMKI

SEQ ID NO: 22 - protein, 220 aa, unidentified
  source 1..220: note = Mustela furo
MNGEASPHPHIKGGPVGEGLHTRGLLLQHQTTRPALGRQSFTMPSPALLCCLVLLAGVGASRHQSALSEDNCTHFPASLPHMLRELRAAFGRVKTFFQMKDKLGSILLTGSLLEDFKGYLGCQALSEMIQFYLEEVMPQAENHDPEVKEHVNSLGEKLKTLRLRLRRCHRFLPCENKSKAVEQVKSAFSKLQERGVYKAMSEFDIFINYIETYMTMRMKI

SEQ ID NO: 23 - protein, 178 aa, Oryctolagus cuniculus
MLSSALLCCLVFLGGTGASRGQDTPAENSCIHFPGGLPHMLRELRAAFGRVKTFFQSKDQLNSMLLTESLLEDLKGYLGCQALSEMIQFYLKDVMPQAENHSPAIREHVNSLGENLKTLRLRLRQCHRFLPCENKSKAVEQVKSAFSKLQEEGVYKAMSEFDIFINYIETYMTMKIKS

SEQ ID NO: 24 - protein, 178 aa, Mesocricetus auratus
MLGSALLCCLLLLAGVGPSRGQYTQHESNCTHFPVSQTHMLRELRTAFSQVKTFFQKKDQLDNILLTDSLLQDFKGYLGCQTLSEMIQFYLVEVMPQAENHGPEIKEHLNSLGEKLKTLRRQLQRCHRFLPCENKSKAVEQVKDNFNKLQEKGVFKAMNEFDIFINCIEVYMTIKMKS

SEQ ID NO: 25 - protein, 178 aa, unidentified
  source 1..178: note = Cricetulus griseus
MPVSALLCCLLLLAGVGPSRGQYTQQENNCTHFPVSQTHMLRELRTAFSQVKTFFQKKDQLDNILLTDSLVQDFEGYLGCQTLSEMIQFYLVEVMPQAENHGPEIKEHLNSLGEKLKTLRRQLQRCHRFLPCENKSKAVEKVKSDFNKLQEKGVYKAMNEFDIFINCIETYMTIKMKS

SEQ ID NO: 26 - protein, 15 aa, synthetic construct
LRRCHRFLPCENKSK

SEQ ID NO: 27 - DNA, 538 bp, synthetic construct
  source 1..538: mol_type = other DNA
atgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatctcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgag

SEQ ID NO: 28 - DNA, 538 bp, Homo sapiens
  source 1..538: mol_type = genomic DNA
atgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgag

(19) United States
(12) Patent Application Publication
(10) Pub. No.: US 2025/0051412 A1
(43) Pub. Date: Feb. 13, 2025

(54) DOSING REGIMEN FOR IL-10 ENCODING EXPRESSION CONSTRUCT

(71) Applicant: Xalud Therapeutics, Inc., Charlottesville, VA (US)

(72) Inventors: Jayson Michael Rieger, Charlottesville, VA (US); David Glover, Afton, VA (US); Howard Rutman, New York, NY (US); Stephen Collins, Lake Forest, IL (US); Diem Nguyen, New York, NY (US)

(73) Assignee: Xalud Therapeutics, Inc., Charlottisville, VA (US)

(21) Appl. No.: 18/720,414

(22) PCT Filed: Dec. 16, 2022
(86) PCT No.: PCT/US2022/081736

§ 371 Date: Jun. 14, 2024

Related U.S. Application Data
(60) Provisional application No. 63/290,092, filed on Dec. 16, 2021.

(51) Int. Cl. C07K 14/54; A61K 38/00; A61K 47/26; A61K 48/00; A61P 19/02; A61P 29/00; C12N 15/86

(52) U.S. Cl. CPC C07K 14/5428; A61K 47/26; A61K 48/0008; A61P 19/02; A61P 29/00; C12N 15/86; A61K 38/00; C12N 2750/14143; C12N 2830/50

(57) ABSTRACT

The present disclosure describes a dosing regimen of an IL-10^{F129S} expression construct for treating an inflammatory joint disease (e.g., osteoarthritis) in a subject. Methods are provided for treating an inflammatory joint disease in a subject by injecting a first dose and second dose of a composition comprising an IL-10^{F129S} expression construct into an inflamed joint of the subject according to a dosing regimen wherein the two doses of the expression construct are administered to a subject approximately 150-360 days apart. The present disclosure also provides kits comprising a first and second dose of an IL-10^{F129S} expression construct. The kits may be useful for treating an inflammatory joint disease in a subject.

### RELATED APPLICATIONS

The present application claims priority under 35 U.S.C. § 119(e) to U.S. provisional application, U.S. Ser. No. 63/290,092, filed Dec. 16, 2021, which is incorporated herein by reference.

### BACKGROUND OF THE INVENTION

Joint pain may flare up in a subject for various reasons, including, for example, as a result of an inflammatory joint disease (e.g., arthritis, such as osteoarthritis (OA)). When injured, a chain of events in the immune system known as the inflammatory cascade is triggered, causing redness, swelling, and pain. Anti-inflammatory cytokines turn off the inflammatory cascade once the threat of injury has diminished. This process, known as local or acute inflammation, is a sign of a healthy immune system. When the inflammatory cascade persists, however, it can lead to chronic inflammation. Treatment with an expression construct encoding IL-10 (such as human IL-10, or a variant thereof, e.g., an IL-10^{F129S} mutant) has been shown to alleviate the symptoms of inflammatory joint disease, as described in U.S. Pat. No. 10,512,672 and U.S. Patent Application Publication No. 2019-0112354, which are incorporated herein by reference.

### SUMMARY OF THE INVENTION

Due to the long-term treatment required to manage inflammatory joint diseases (e.g., osteoarthritis (e.g., osteoarthritis of the knee)), clinical studies are needed to optimize the dosing regimen for IL-10-encoding expression constructs. Such improvements could significantly improve the quality of life for patients with inflammatory joint disease. Previous work has demonstrated that an expression construct (e.g., a plasmid) encoding a variant of IL-10 (e.g., human IL-10 comprising an F129S mutation) shows positive results when injected into inflamed joints (e.g., knee) of patients suffering from osteoarthritis. In particular, XT-150, a specific expression construct encoding IL-10^{F129S} described in detail hereinafter, has shown promising efficacy. Unlike other pain medications, XT-150 is not a short-acting analgesic but rather is “disease-altering” at the site of inflammation. The sustained release of IL-10^{F129S} seeks to restore cytokine homeostasis and inflammatory balance in the knee. This mechanism of action delivers a long-lasting benefit to patients, and thus it sought to determine whether a second dose maintained or improved efficacy from the first dose. In order to evaluate the safety and efficacy of XT-150, a randomized, double-blind, placebo-controlled Phase 2 study of XT-150 was conducted in patients with moderate-to-severe pain due to osteoarthritis of the knee. A benefit of repeat dosing was observed across a broad population of patients with moderate-to-severe osteoarthritis. A prolonged benefit of at least 360 days was observed. These patients were administered two doses of either 150 μg or 450 μg of XT-150 by injection into the knee space approximately 180-210 days apart (and up to 330 days apart). Patients with a baseline WOMAC pain score of greater than 9 receiving two injections of the IL-10^{F129S} expression construct (XT-150) showed statistically significant improvement compared to WOMAC pain scores observed in patients administered a single active dose. Enhanced improvements in WOMAC function scores were also observed with repeat dosing at approximately 180-210 days with statistical significance. Finally, administration of XT-150 showed an exemplary safety profile, and no drug related serious adverse events were observed in the 286 patients dosed. No adverse immune response to IL-10 (e.g., no production of IL-10 antibodies) was observed up to day 360.

Thus, in one aspect provided herein is a method for treating an inflammatory joint disease in a subject, the method comprising injecting into an inflamed joint of the subject a first dose of a composition comprising an IL-10^{F129S} expression construct, wherein the first dose of the composition comprises 100 μg to 600 μg of the IL-10^{F129S} expression construct; and injecting into the inflamed joint of the subject a second dose of the composition 150-360 days after injection of the first dose, wherein the second dose of the composition comprises 100 μg to 600 μg of the IL-10^{F129S} expression construct. Any subject with inflammatory joint disease may benefit from repeat dosing, using any of the dosing regimens described herein.

In some embodiments, the second dose of the composition is injected 150 or more days after injection of the first dose. In some embodiments, the second dose of the composition is injected about 180 days after injection of the first dose. In some embodiments, the first dose comprises about 150 μg of the IL-10^{F129S} expression construct, and the second dose comprises about 450 μg of the IL-10^{F129S} expression construct. In some embodiments, the first dose comprises about 450 μg of the IL-10^{F129S} expression construct, and the second dose comprises about 450 μg of the IL-10^{F129S} expression construct. In some embodiments, the second dose of the composition is injected 150 or more days after injection of the first dose. In some embodiments, the second dose of the composition is injected about 180 days after injection of the first dose. In some embodiments, the first dose comprises about 150 μg of the IL-10^{F129S} expression construct, and the second dose comprises about 450 μg of the IL-10^{F129S} expression construct. In some embodiments, the first dose comprises about 450 μg of the IL-10^{F129S} expression construct, and the second dose comprises about 450 μg of the IL-10^{F129S} expression construct. In some embodiments, the first and the second dose of the composition are formulated in a solution of phosphate-buffered saline (pH 7.4) with 3% w/v sucrose and 2.5 mg-mL D-mannose. In some embodiments, the composition comprises about 0.15-0.45 mg/mL of IL-10^{F129S} expression construct (e.g., 0.15 or 0.45 mg/mL). In some embodiments, 1 mL of composition is injected into the inflamed joint. In some embodiments, the inflammatory joint disease is osteoarthritis. In some embodiments, the inflammatory joint disease is osteoarthritis of the knee.

In another aspects, provided herein is a method for treating an inflammatory joint disease in a subject, comprising: injecting into an inflamed joint of the subject a first dose of a composition comprising an IL-10^{F129S} expression construct, wherein the first dose of the composition comprises 450 μg of the IL-10^{F129S} expression construct; and injecting into the inflamed joint of the subject a second dose of the composition about 150-180 days after injection of the first dose, wherein the second dose of the composition comprises 450 μg of the IL-10^{F129S} expression construct. In some embodiments, the second dose is injected about 180 days after the first dose. In some embodiments, the first and the second dose of the composition are formulated in a solution of phosphate-buffered saline (pH 7.4) with 3% w/v sucrose and 2.5 mg-mL D-mannose. In some embodiments, the composition comprises about 0.45 mg/mL of IL-10^{F129S} expression construct. In some embodiments, the inflammatory joint disease is osteoarthritis. In some embodiments, the inflammatory joint disease is osteoarthritis of the knee.

In another aspect, provided herein is the use of a composition comprising an IL-10^{F129S} expression construct for the treatment of an inflammatory joint disease, wherein the composition is for administration in a first dose comprising 150 μg or 450 μg of the IL-10^{F129S} expression construct, and wherein the composition is for administration in a second dose comprising 450 μg of the IL-10^{F129S} expression construct 150-360 days after the first dose.

Also provided herein are kits. In some embodiments, the kit comprises a first dose and a second dose of an IL-10^{F129S} expression construct, wherein the first dose comprises 150 μg or 450 μg of the expression construct, and wherein the second dose comprises 450 μg of the expression construct; and wherein the first and/or the second dose of the composition is formulated in a sterile solution of phosphate-buffered saline (pH 7.4) with about 3% w/v sucrose and about 2.5 mg/mL D-mannose.

The details of non-limiting embodiments of the disclosure are set forth in the Detailed Description of Certain Embodiments, as described below. Other features, objects, and advantages of the invention will be apparent from the Definitions, Figures, Examples, and Claims.

### BRIEF DESCRIPTION OF THE DRAWINGS

The following drawings provide non-limiting examples of the invention.

FIGS. 1A-1B show WOMAC pain (FIG. 1A) and function (FIG. 1B) responders of a population of targeted patients (baseline 9-14 WOMAC pain and KL3) at day 180. In the target population, a 450 μg dose shows separation from placebo at 50% responder threshold.

FIG. 2 shows WOMAC pain and function 50% threshold in stage A/stage B of a population of targeted patients (baseline 9-14 WOMAC pain and KL 2/3) at day 360. A double dose of IL-10^{F129S} expression construct was found to be significantly better than a single dose. A repeat dose of 450 μg was also found to be significantly better than a single dose of 450 μg.

FIG. 3 shows WOMAC pain responders in stage B of a population of targeted patients (baseline 9-14 WOMAC pain and KL 2/3) at day 360. Patients that received a double dose of IL-10^{F129S} expression construct showed significant pain reduction over a single dose.

FIG. 4 shows that WOMAC function responder rate is also significantly differentiated in patients receiving a double dose vs. a single dose of XT-150.

FIGS. 5A-5B show stage B WOMAC pain (FIG. 5A) and WOMAC function (FIG. 5B) responders from a broad population of patients (baseline 9+ WOMAC pain and KL 2/3) at day 360.

FIGS. 6A-6B show stage B WOMAC pain (FIG. 6A) and WOMAC function (FIG. 6B) responders from a population of targeted patients (baseline 9-14 WOMAC pain and KL 2/3) at day 360.

FIG. 7 is a plasmid map of XT-150.

FIG. 8 shows the change in pain for subjects at different dosages, including when compared to a subset of responders with ≥30% reduction in pain.

FIG. 9 shows the percent reduction in pain score for subjects at different dosages, including when compared to a subset of responders with ≥30% reduction in pain. Both FIG. 8 and FIG. 9 utilized control with either a true placebo (saline) or a pseudo-placebo (sub therapeutic dose of 15 μg). The placebo/15 μg was compared as a cohort to either 150 μg or 450 μg doses. To gain further insight, for those patients receiving 150 μg or 450 μg who achieved at least a 30% reduction from their baseline WOMAC pain, they were considered “responders”. Those subjects are also presented as a subset labeled “resp” to show that for those patients that did respond, the response was quite robust. As shown, irrespective of dose, there was an improvement of WOMAC pain and function with XT-150.

FIG. 10 shows change in WOMAC pain for patients with WOMAC baseline pain between 1-20 with a repeat dose of XT-150 compared to a single dose over 360 days.

FIG. 11 shows change in WOMAC function for patients with WOMAC baseline pain between 1-20 with a repeat dose of XT-150 compared to a single dose over 360 days.

FIG. 12 shows change in WOMAC pain for patients with WOMAC baseline pain between 9-20 with a repeat dose of XT-150 compared to a single dose over 360 days.

FIG. 13 shows change in WOMAN function for patients with WOMAC baseline pain between 9-20 with a repeat dose of XT-150 compared to a single dose over 360 days.

FIG. 14 shows change in WOMAC pain for patients with WOMAC baseline pain between 9-20 with a repeat dose of XT-150 compared to a single dose over 180 days.

FIG. 15 shows change in WOMAN function for patients with WOMAC baseline pain between 9-20 with a repeat dose of XT-150 compared to a single dose over 180 days.

FIG. 16 shows change in WOMAC pain for patients with WOMAC baseline pain between 9-20 over 360 days with either a single or repeat dose.

FIG. 17 shows the least square mean difference at day 360 of FIG. 16 demonstrating a change from baseline difference of −2.8 points, p=0.0081, and a strong effect size of 0.81 in WOMAC pain.

FIG. 18 shows change in WOMAC function for patients with WOMAC baseline pain between 9-20 over 360 days with either a single or repeat dose.

FIG. 19 shows the least square mean difference at day 360 of FIG. 18 demonstrating a change from baseline difference of −9.6 points, p=0.0101, and a strong effect of 0.71 in WOMAC function.

FIG. 20 shows the least square mean change from baseline of WOMAC pain at day 360 for a single or repeat dose of 450 μg.

FIG. 21 shows the least square mean change from baseline of WOMAC function at day 360 for a single or repeat dose of 450 μg.

FIG. 22 shows the least square mean change from baseline of patient overall assessment at day 360 for a single or repeat dose of 450 μg.

FIG. 23 shows the change in knee range of motion at day 360 with either a single or repeat dose of 450 μg.

FIG. 24 shows the change in walking speed at day 360 with either a single or repeat dose of 450 μg.

FIG. 25 shows a reference total knee arthroplasty (TKA) Gait Changes for both walking speed and flexion range.

FIG. 26 shows the least square meant change in WOMAC pain over 360 days with either a single dose of 450 μg or 150 μg, or repeat dose with either 450 μg and 450 μg, 150 μg and 150 μg, 450 μg and 150 μg, or 150 μg and 450 μg.

FIG. 27 shows the change in WOMAC pain from baseline at day 360 after second injection based on the data in FIG. 26.

FIG. 28 shows the pain response for XT-150 compared to products currently on the market.

FIG. 29 shows the WOMAC pain change from baseline least square mean difference at day 360 for a single dose at either 450 μg or 150 μg, or repeat dose with either 450 μg and 450 μg, 150 μg and 150 μg, 450 μg and 150 μg, or 150 μg and 450 μg.

FIG. 30 shows the WOMAC function change from baseline least square mean difference at day 360 for a single dose at either 450 μg or 150 μg, or repeat dose with either 450 μg and 450 μg, 150 μg and 150 μg, 450 μg and 150 μg, or 150 μg and 450 μg.

FIG. 31 shows the least square mean difference for WOMAC pain score through day 360 for the single dose of 450 μg compared to the repeat doses of 450 μg.

FIG. 32 shows the least square mean difference for WOMAC function score through day 360 for the single dose of 450 μg compared to the repeat doses of 450 μg.

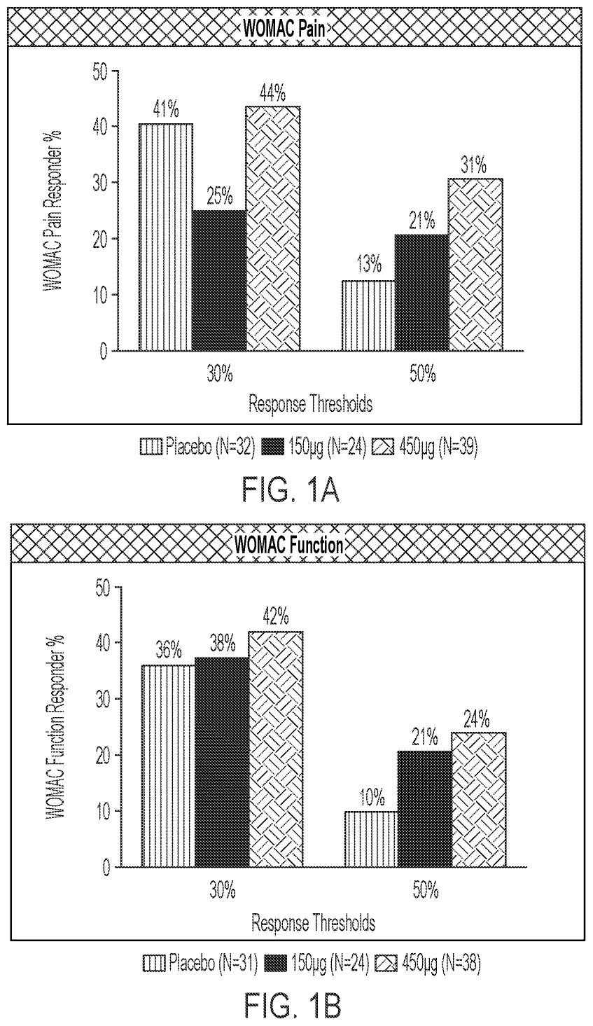

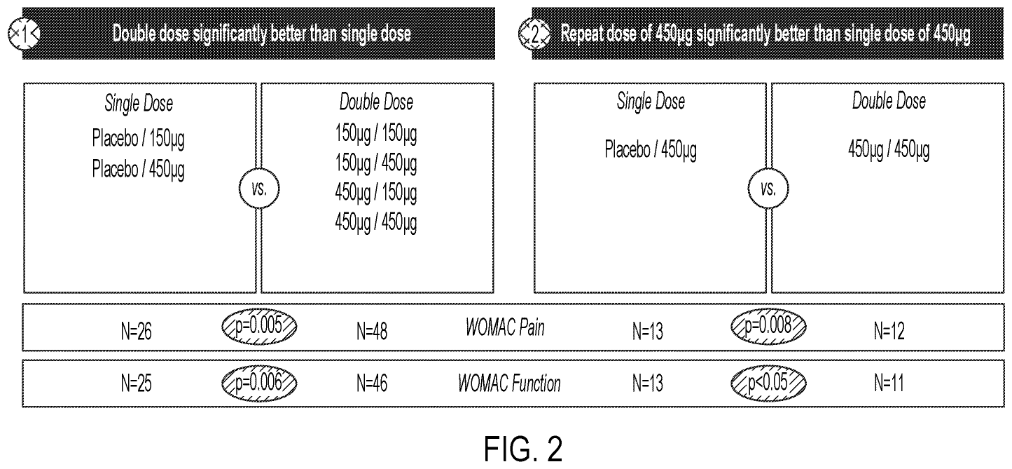

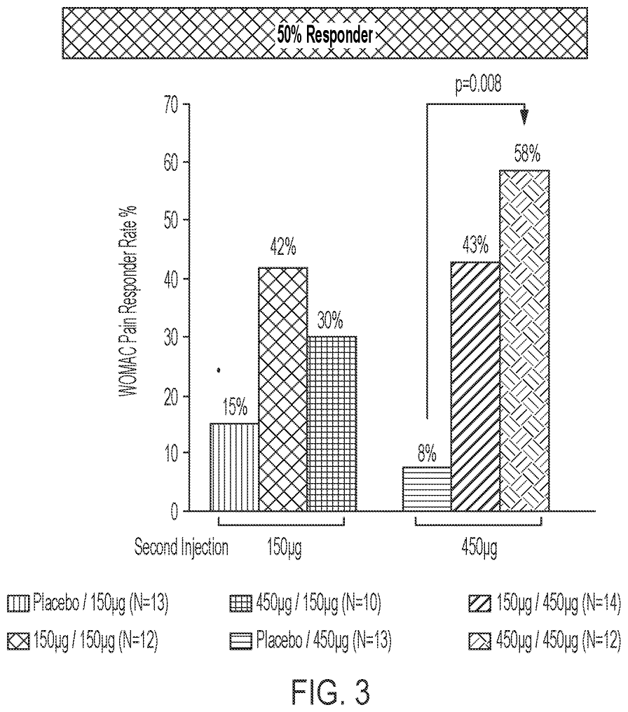

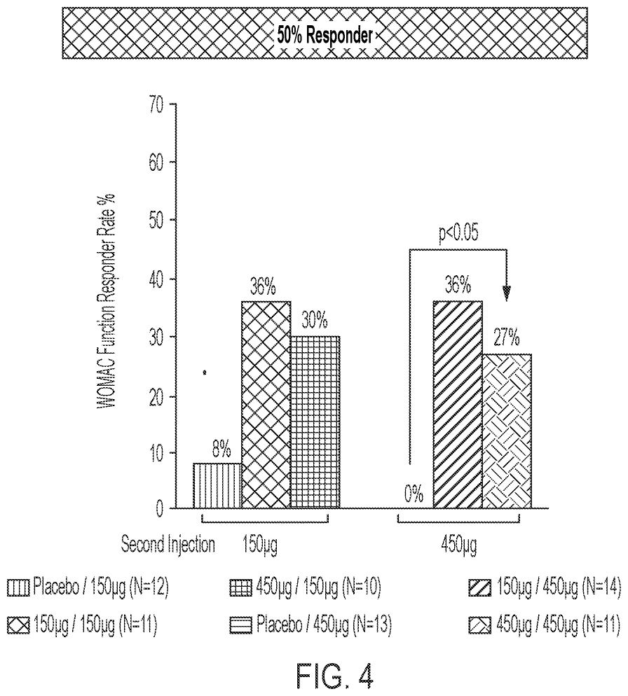

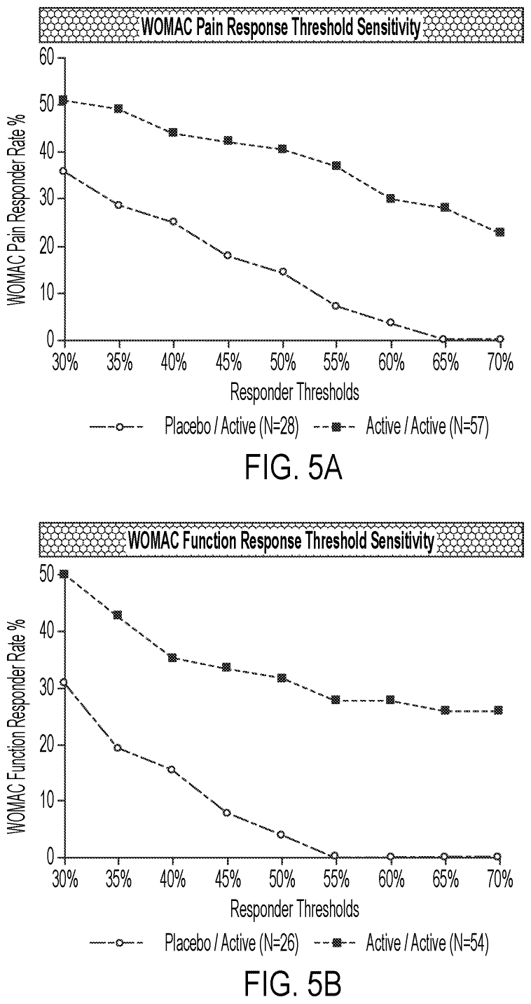

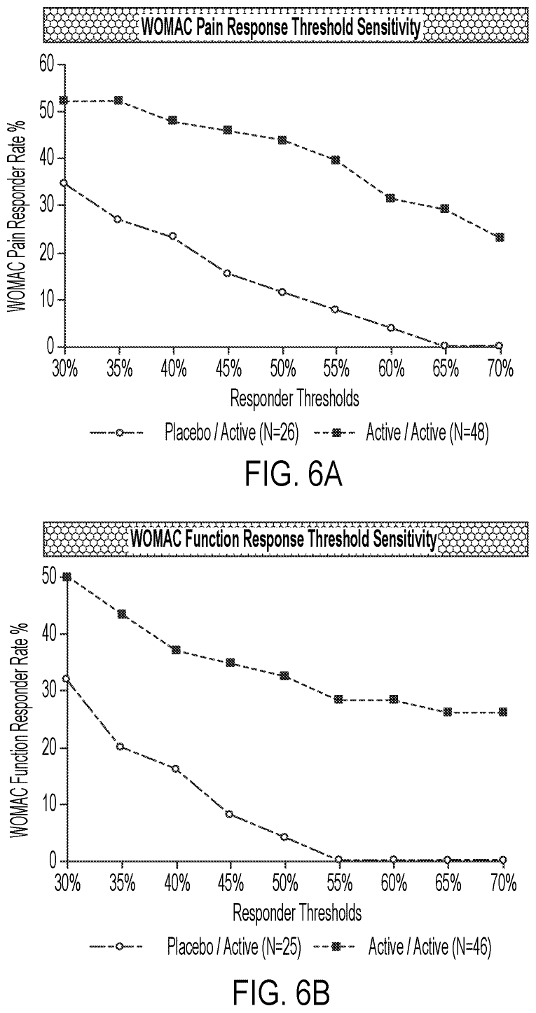

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

The term “about X” or “approximately X,” where X is a number or percentage, refers to a number or percentage that is between 99.5% and 100.5%, between 99% and 101%, between 98% and 102%, between 97% and 103%, between 96% and 104%, between 95% and 105%, between 92% and 108%, or between 90% and 110%, inclusive, of X. When using the term “about” with respect to the number of days, about refers to +/−2 days, 3 days, 4 days, 5 days, 7 days, 10 days, or 14 days. In some embodiments, when using the term “about” with respect to the number of days, about refers to +/−2 days, 3 days, 4 days, 5 days, or 7 days. In some embodiments, when using the term “about” with respect to the number of days, about refers to +/−2 days, 3 days, or 4 days.

The term “excipient that increases the uptake” of another agent, as used herein, refers to a pharmacological or immunological agent that modifies the effect of other agents. In the context of the present application, an excipient that increases the uptake of a vector is used to increase the efficacy of the therapeutic IL-10^{F129S} expression construct used in the dosing regimen described herein. In some embodiments, such excipients include those that enhance the uptake or efficacy of the IL-10^{F129S} expression construct by, e.g., macrophages or other immune cells present in the synovial fluid of the joint. In some embodiments, such an excipient is selected from the group consisting of a sugar, calcium phosphate, a dendrimer, an oligonucleotide, a high molecular weight hyaluronic acid, and a lipid. In some embodiments, the excipient is D-mannose, sucrose, or glucose. In certain embodiments, the excipient is D-mannose. In certain embodiments, a composition provided herein comprises D-mannose. In certain embodiments, a composition provided herein comprises about 2.5 mg/mL D-mannose. In some embodiments, a composition provided herein further comprises an additional sugar. In some embodiments, a composition provided herein comprises mannose and an additional sugar. In some embodiments, a composition provided herein comprises D-mannose and an additional sugar. In certain embodiments, the additional sugar is sucrose. In some embodiments, a composition provided herein comprises mannose and sucrose. In some embodiments, a composition provided herein comprises about 2.5 mg/mL D-mannose and about 3% w/v sucrose.

The term “administer,” “administering,” or “administration” refers to implanting, absorbing, ingesting, injecting, inhaling, or otherwise introducing a composition described herein into a subject. In certain embodiments, “administering” a composition described herein refers to injecting the composition into a joint (e.g., an inflamed joint). In certain embodiments, “administering” a composition described herein refers to intra-articular (IA) administration. In certain embodiments, administration refers to intrathecal injection (e.g., injection into the spinal canal, or into the subarachnoid space such that the administered composition reaches the cerebrospinal fluid). In some embodiments, administration refers to transforaminal injection, intraventricular injection, or intraparenchymal injection.

The term “anti-inflammatory” as used herein, refers to decreasing the action or production of one or more proinflammatory cytokines (i.e., signaling molecules that are secreted from immune cells and promote inflammation) or proteins produced by nerves, neurons, glial cells, endothelial cells, fibroblasts, muscle, immune cells, or other cell types.

The term DNA “control sequences” refers collectively to promoter sequences, polyadenylation signals, transcription termination sequences, upstream regulatory domains, origins of replication, internal ribosome entry sites, enhancers, and the like, which collectively provide for the replication, transcription, and translation of a coding sequence in a recipient cell. The vectors provided herein contain one or more control sequence. Notably, not all types of control sequences need to be present, as long as the selected coding sequence is capable of being replicated, transcribed, and translated in an appropriate host cell.

The term “excipient” or “diluent” refers to a substance added to a therapeutic composition to facilitate administration of the therapeutic IL-10^{F129S} expression constructs provided using the dosing regimen described herein. In certain embodiments, the excipient or diluent is inert. Examples, without limitation, of excipients include saline, calcium carbonate, calcium phosphate, various sugars and types of starch, cellulose derivatives, gelatin, hyaluronic acid (optionally formulated with a surfactant), Plumoic F-68, vegetable oils, and polyethylene glycols. Further examples of diluents and excipients are provided herein. In some embodiments, the diluent is phosphate-buffered saline (pH 7.4).

The term “joint” refers to an anatomical structure where two bones meet, including the ligaments that connect the bones to one another, the tendons that attach muscles to the bones, and the joint capsule, bursae, and synovium. Joints that can be treated using the methods provided herein include fixed, hinge, pivot, and ball-and-socket joints. In some embodiments, the inflamed joint is a hand, a foot, a neck, a hip, a knee, an elbow, a wrist, an ankle, a hip, a shoulder, a jaw, or a spine (including, e.g., the facet joint). In some embodiments, a joint is a knee, an elbow, a wrist, an ankle, a hip, a shoulder, a jaw, or a spine (including, e.g., the facet joint). In some embodiments, a joint is inflamed.

The term “joint inflammation” refers to all types of arthritis caused by inflammation. Rheumatoid arthritis and osteoarthritis are the most common forms of joint inflammation. Other types of joint inflammation include tendonitis, bursitis, inflammation of the ligament, synovitis, gout, facet syndrome, and systemic lupus erythematosus. In certain embodiments, the joint inflammation is osteoarthritis.

The “Kellgren-Lawrence” grading scale refers to a system for classifying the severity of osteoarthritis in a patient. Patients are classified as grade 0 (no symptoms of osteoarthritis), 1 (likely no symptoms of osteoarthritis), 2 (minimal osteoarthritis), 3 (moderate osteoarthritis), or 4 (severe osteoarthritis).

The term “nuclear targeting sequence” refers to a nucleic acid sequence that functions to improve the expression efficiency of an anti-inflammatory cytokine (e.g., IL-10) in a cell.

The terms “polynucleotide”, “nucleotide sequence”, “nucleic acid”, “nucleic acid molecule”, “nucleic acid sequence”, and “oligonucleotide” refer to a series of nucleotide bases (also called “nucleotides”) in DNA and RNA (i.e., any chain of two or more nucleotides). The polynucleotides can be chimeric mixtures or derivatives or modified versions thereof, single-stranded or double-stranded. The oligonucleotide can be modified at the base moiety, sugar moiety, or phosphate backbone, for example, to improve stability of the molecule, its hybridization parameters, etc. A nucleotide sequence typically carries genetic information, including the information used by cellular machinery to make proteins and enzymes. In some embodiments, a nucleotide sequence encodes IL-10^{F129S} as described herein. A nucleotide sequence (e.g., for encoding IL-10^{F129S}) may be placed under the control of a promoter in an expression construct, such as a vector. Vectors can be plasmid, viral, or others known in the art, used for replication and expression in cells. Expression of the sequence encoding IL-10^{F129S} can be facilitated by any promoter known in the art to act in mammalian, preferably human, cells. Such promoters can be inducible or constitutive. Any type of plasmid, cosmid, yeast artificial chromosome, or viral vector can be used to prepare the recombinant expression construct that can be introduced directly into the tissue site.

The polynucleotides may be flanked by natural regulatory (expression control) sequences or may be associated with heterologous sequences, including promoters, internal ribosome entry sites (IRES), and other ribosome binding site sequences, enhancers, response elements, suppressors, signal sequences, polyadenylation sequences, introns, 5′- and 3′-non-coding regions, and the like. In some embodiments, the polynucleotides are flanked by inverted terminal repeat (ITR) sequences. In some embodiments, an ITR sequence is an adeno-associated virus (AAV) ITR sequence.

“Operably linked” refers to an arrangement of elements where the components so described are configured so as to perform their usual function. Thus, control sequences operably linked to a coding sequence are capable of effecting the expression of the coding sequence. The control sequences need not be contiguous with the coding sequence so long as they function to direct the expression thereof. Thus, for example, intervening untranslated yet transcribed sequences can be present between a promoter sequence and the coding sequence, and the promoter sequence can still be considered “operably linked” to the coding sequence.

The term “promoter” is used herein in its ordinary sense to refer to a nucleotide region comprising a DNA regulatory sequence, wherein the regulatory sequence is derived from a gene that is capable of binding RNA polymerase and initiating transcription of a downstream (3′-direction) coding sequence. Transcription promoters can include “inducible promoters” (where expression of a polynucleotide sequence operably linked to the promoter is induced by an analyte, cofactor, regulatory protein, etc.), “repressible promoters” (where expression of a polynucleotide sequence operably linked to the promoter is induced by an analyte, cofactor, regulatory protein, etc.), and “constitutive promoters.” In some embodiments, promoters may be chicken or human β-actin promoters, CAG hybrid promoter, cytomegalovirus immediate early promoters, glyceraldehyde 3-phosphate dehydrogenase (GAPDH) promoters, elongation factor 1α (eF1α) promoters, GFAP promoters, murine leukemia virus (MLV) promoters, herpes simplex virus thymidine kinase (TK) promoters, and woodchuck hepatitis virus post-transcriptional regulatory element (WPRE) promoters. In certain embodiments, a promoter is a cytomegalovirus immediate early promoter. In some embodiments, the promoter (e.g., constitutive promoter) is selected from the group consisting of ubiquitin promoters, CMV promoters, β-actin promoters, CAG hybrid promoters, histone H4 promoters, EF-1α promoters, PGK gene promoters, promoter elements controlled by RNA polymerase II, promoter elements controlled by RNA polymerase I, promoter elements controlled by RNA polymerase III, U6 promoters (e.g., U6-1 promoters, U6-8 promoters, U6-9 promoters), H1 promoter, 7SL promoter, human Y promoters (e.g., hY1 promoters, hY3 promoters, hY4 promoters, and hY5 promoters), human MRP-7-2 promoter, adenovirus VA1 promoter, human tRNA promoters, and 5s ribosomal RNA promoters. In some embodiments the promotor is a chicken or human β-actin promoter, CAG hybrid promoter, cytomegalovirus immediate early promoter, or elongation factor 1α (eF1α) promoter. In some embodiments the promotor is a chicken β-actin promoter. In some embodiments the promotor is a CAG hybrid promoter. In some embodiments the promotor is a CAG hybrid promoter. In some embodiments the promotor is a eF1α promoter.

For the purpose of describing the relative position of nucleotide sequences in a particular nucleic acid molecule throughout the instant application, such as when a particular nucleotide sequence is described as being situated “upstream,” “downstream,” “3 prime (3′),” or “5 prime (5)” relative to another sequence, it is to be understood that it is the position of the sequences in the “sense” or “coding” strand of a DNA molecule that is being referred to, as is conventional in the art.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, camel, cat, or dog), or bird (e.g., commercially relevant bird, such as chicken, duck, goose, or turkey)). The non-human animal may be a male or female at any stage of development. The non-human animal may be a transgenic animal or genetically engineered animal. The term “patient” refers to a human subject in need of treatment of a disease (e.g., an inflammatory joint disease, such as osteoarthritis).

The terms “treatment,” “treat,” and “treating” refer to reversing, alleviating, delaying the onset of, or inhibiting the progress of a disease described herein (e.g., an inflammatory joint disease, such as osteoarthritis). In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms). Treatment may also be continued after symptoms have resolved, for example, to delay or prevent recurrence. “Treatment” or “treating” joint inflammation may include: (1) decreasing inflammation of the joint or causing the inflammation to occur with less intensity in a subject that may be predisposed to joint inflammation but does not yet experience or display symptoms, or (2) inhibiting joint inflammation, i.e., arresting the development of or reversing symptoms or physiological damage caused by inflammation. “Treatment” or “treating” joint inflammation may also refer to reducing pain associated with joint inflammation experienced by a patient, and/or improving the function of the joint. In some embodiments, the methods provided herein are methods for treating pain associated with inflammatory joint disease in a subject.

The Western Ontario and McMaster Universities Osteoarthritis Index (WOMAC) is a set of criteria used by health professionals to evaluate the condition of patients with osteoarthritis of the knee and hip. Criteria include pain, stiffness, and physical functioning of the joints. Higher scores indicate worse pain (score range 0-20), stiffness (score range 0-8) and functional limitations (score range 0-68). Patients with a WOMAC pain score of greater than 9, or 9-20, may be considered to have moderate-to-severe osteoarthritis. WOMAC is discussed in more detail, for example, in Quintana, J. M. et al. Arch. Intern. Med. 2006, 166(2), 220-226.

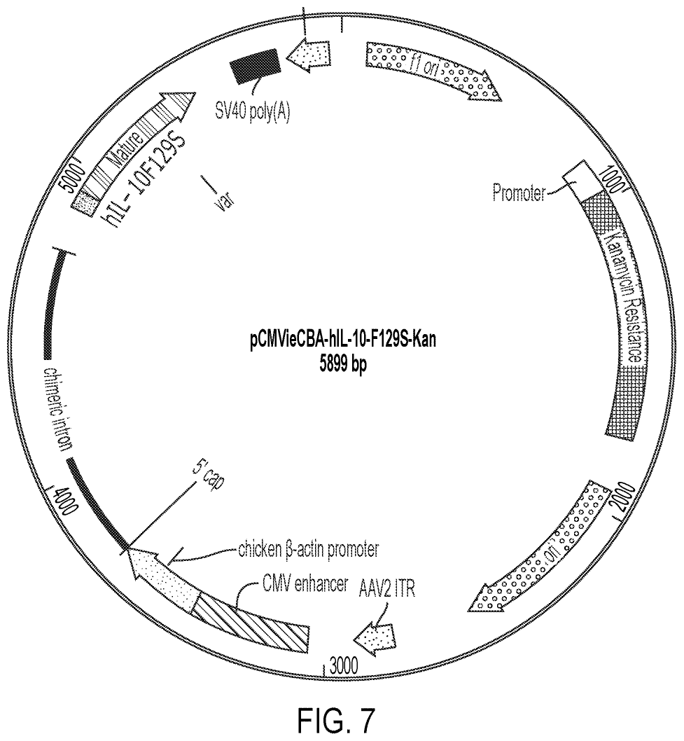

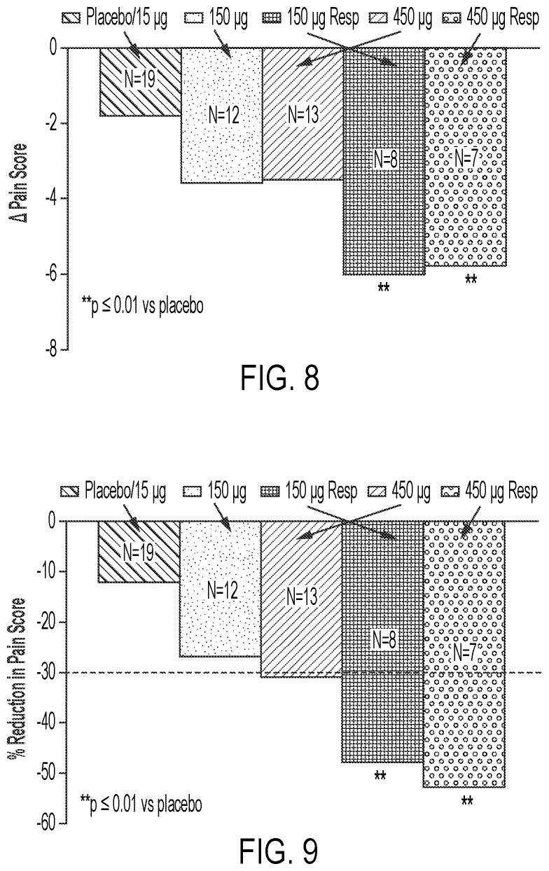

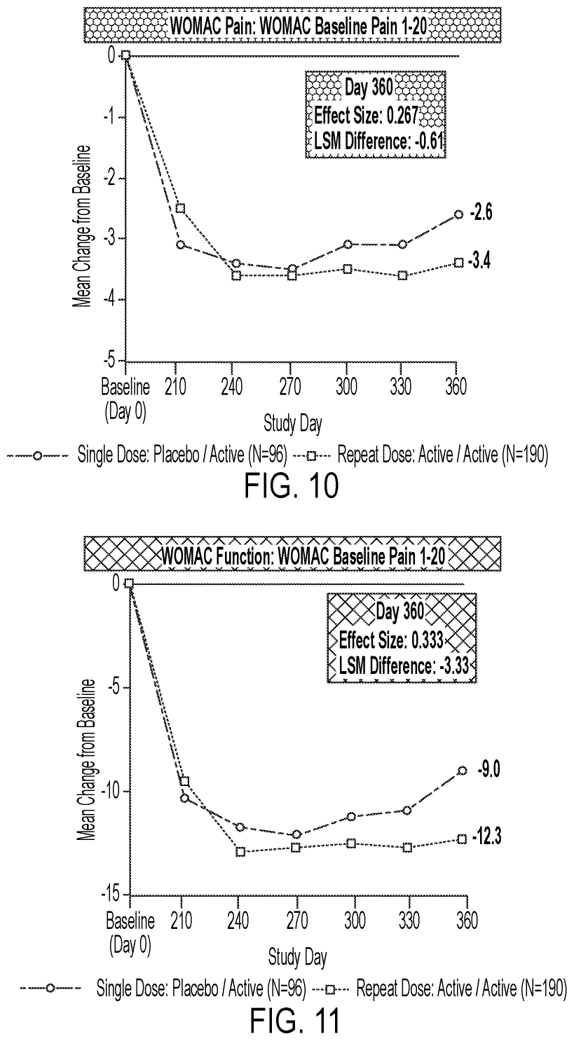

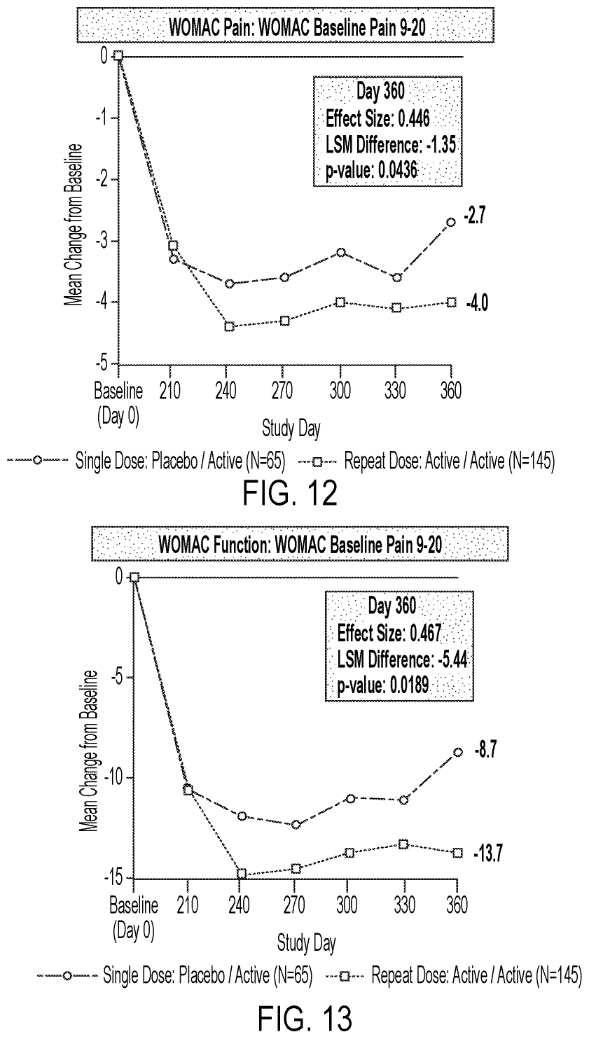

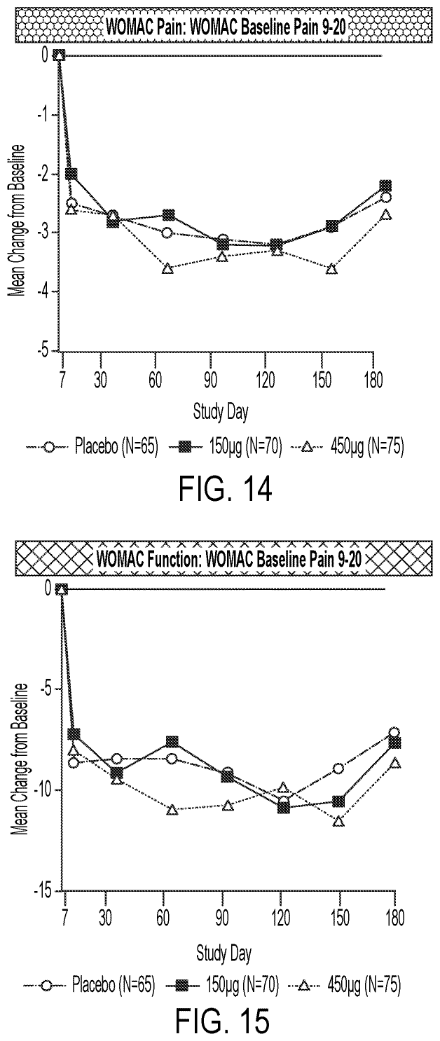

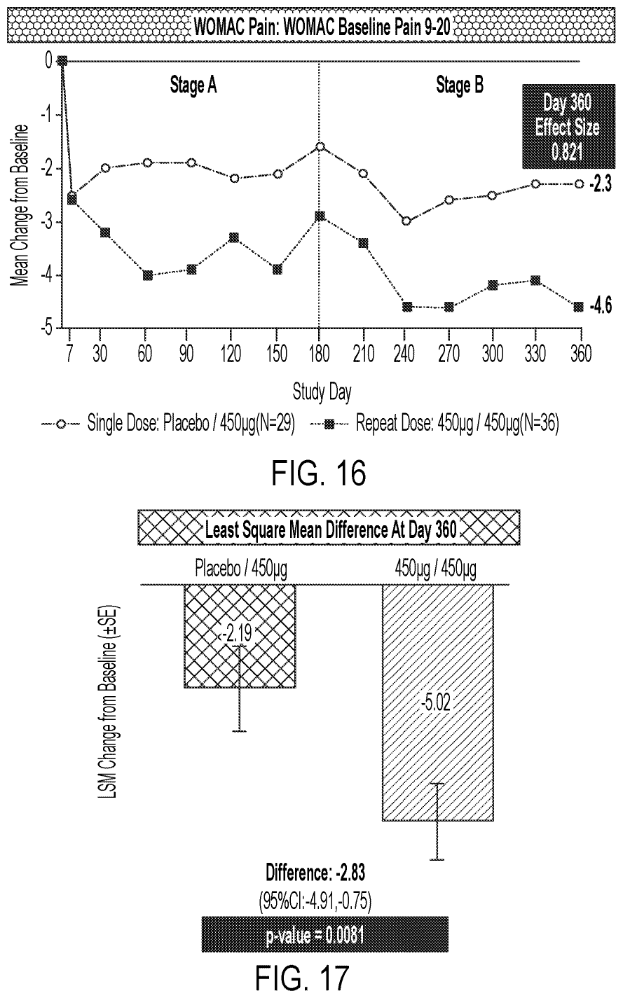

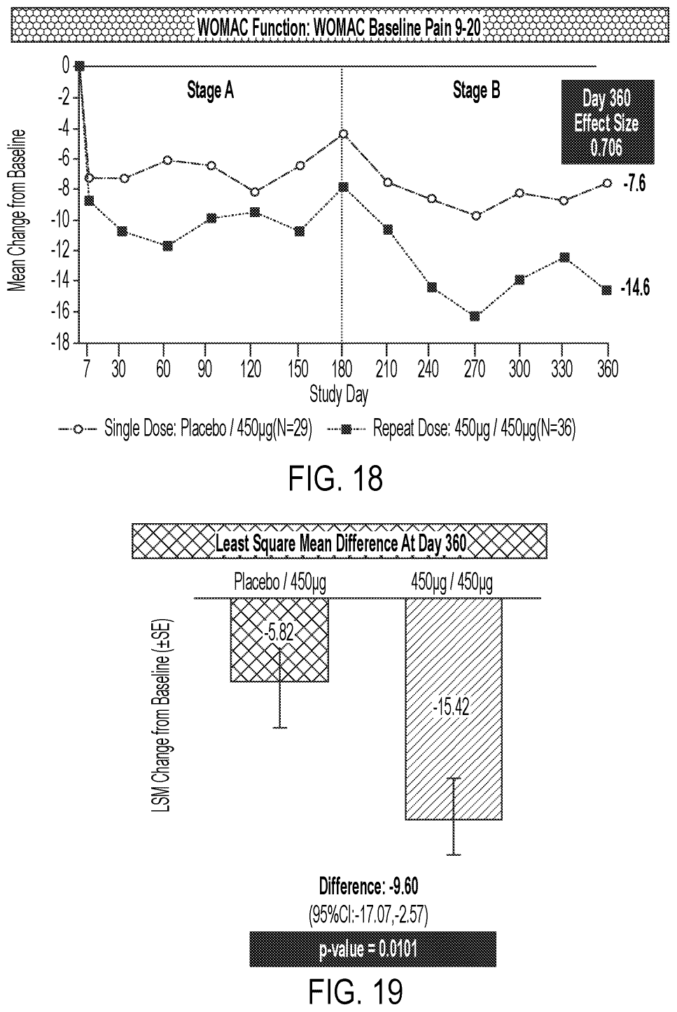

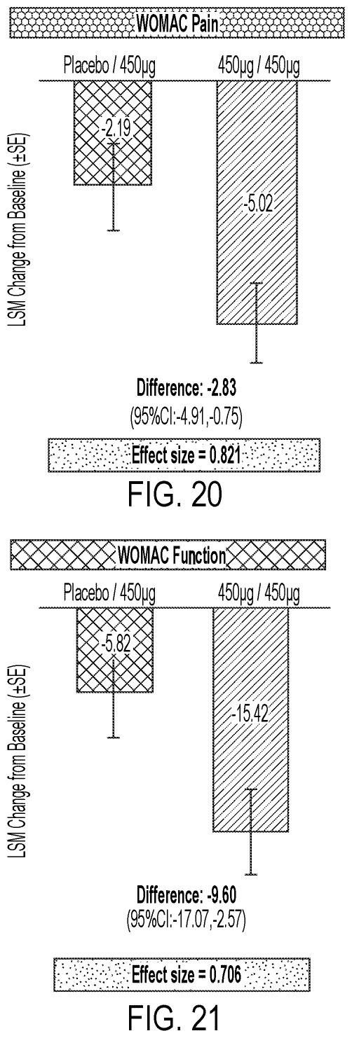

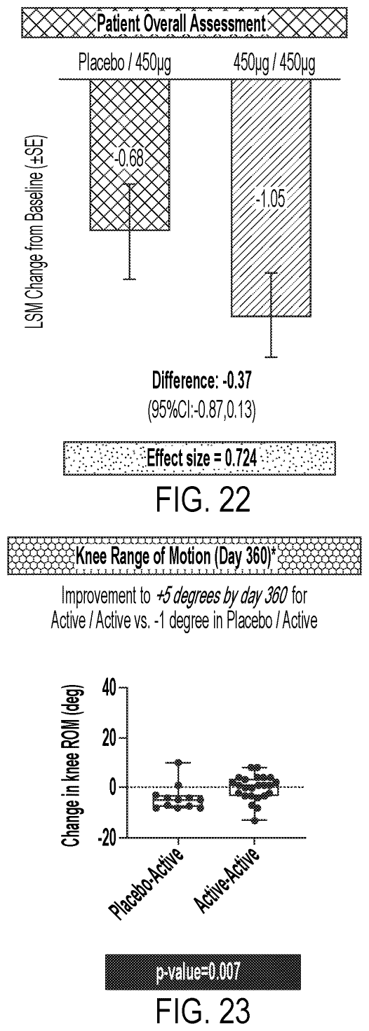

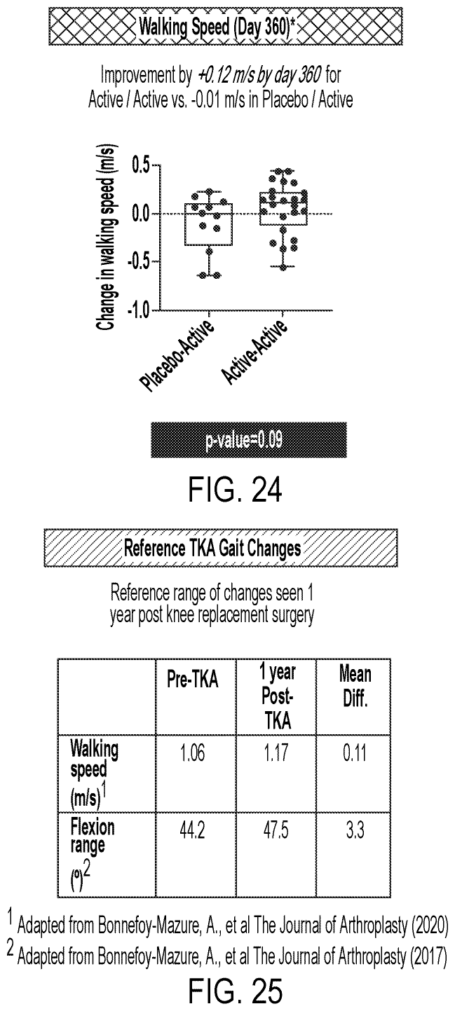

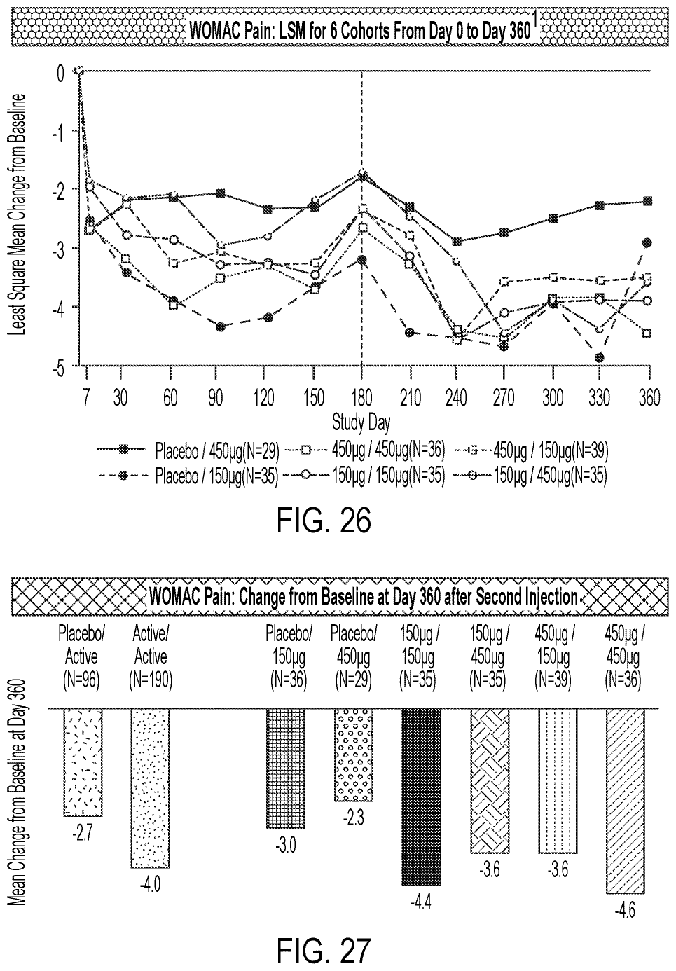

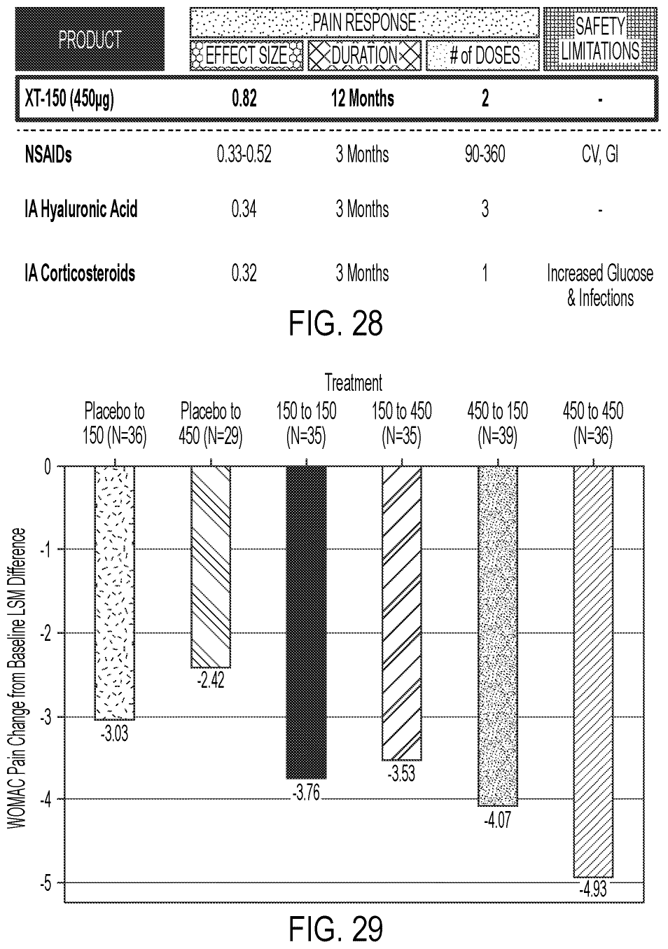

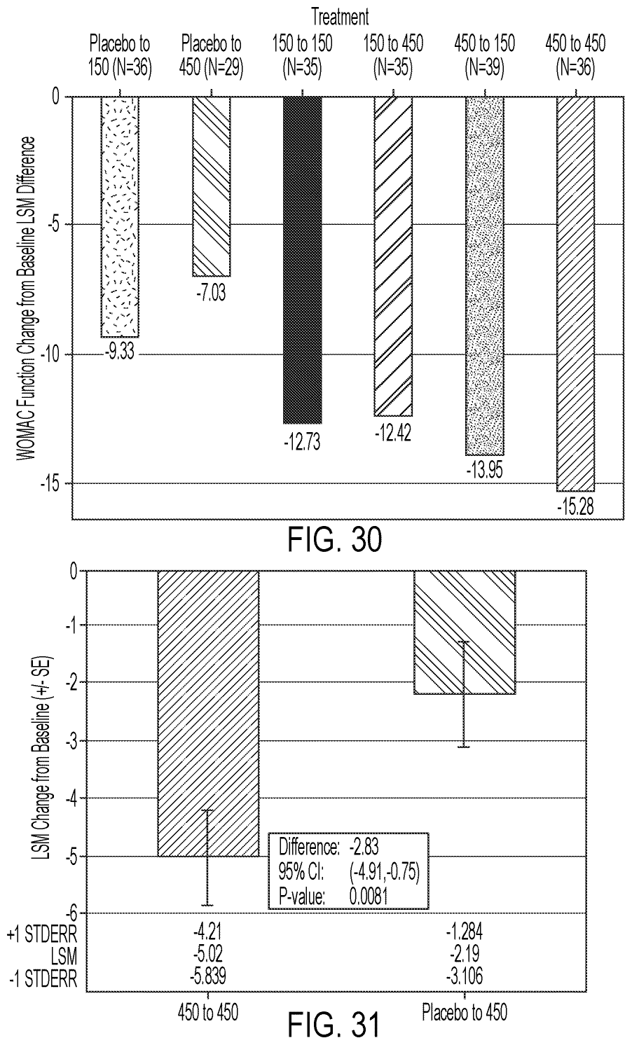

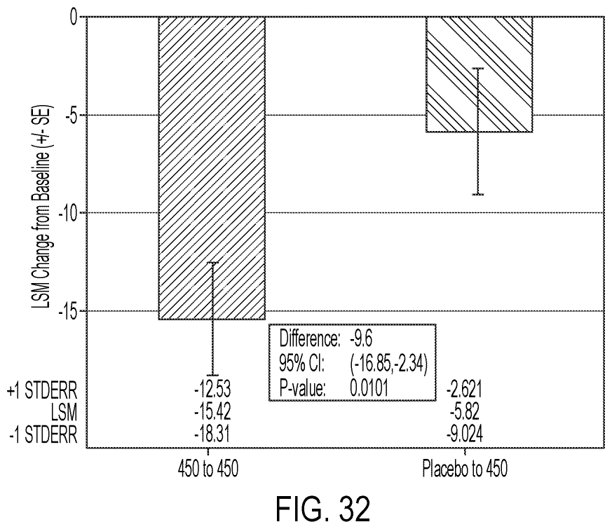

The term “XT-150” refers to a circular vector or plasmid of SEQ ID NO: 1 as provided herein, and as shown in FIG. 7, which encodes IL-10^{F129S}. In some embodiments, XT-150 is provided in a composition comprising phosphate-buffered saline (pH 7.4), about 2.5 mg/mL D-mannose, and about 3% w/v sucrose. In some embodiments, the concentration of XT-150 is between 0.1-0.6 mg/mL. In some embodiments, the concentration of XT-150 is between 0.15-0.45 mg/mL. In some embodiments, the concentration of XT-150 is 0.15 or 0.45 mg/mL. The composition volume delivered depends on the joint to which the composition is administered. When delivered to a knee, the composition may be approximately 1 mL.

The term “XT-151” refers to a circular vector or plasmid of SEQ ID NO: 2 as provided herein which encodes IL-10^{F129S} and IL-10R1.

### DETAILED DESCRIPTION

The present disclosure is based on the development of a dosing regimen for treating inflammatory joint diseases in a subject. It was discovered that administering a first dose of a composition comprising an IL-10^{F129S} expression construct to a subject, followed by administration of a second dose approximately four to six months later, significantly reduces pain associated with osteoarthritis in the subject. Accordingly, the present disclosure provides methods for treating an inflammatory joint disease, such as, e.g., osteoarthritis, in a subject by injecting a first dose and second dose of a composition comprising an IL-10^{F129S} expression construct into an inflamed joint of the subject according to the dosing regimen described herein. Additionally, the present disclosure provides for the use of an IL-10^{F129S} expression construct for the treatment of an inflammatory joint disease. The present disclosure also provides kits comprising a first and second dose of an IL-10^{F129S} expression construct. The kits may be useful for treating an inflammatory joint disease in a subject.

Thus, in one aspect, provided herein are methods for treating an inflammatory joint disease in a subject, the method comprising: injecting into an inflamed joint of the subject a first dose of a composition comprising an IL-10^{F129S} expression construct, wherein the first dose of the composition comprises 100 μg to 600 μg of the IL-10^{F129S} expression construct; and injecting into the inflamed joint of the subject a second dose of the composition 150-360 days after injection of the first dose, wherein the second dose of the composition comprises 100 μg to 600 μg of the IL-10^{F129S} expression construct.

In some embodiments, the second dose of the composition is injected about 150-270 days, about 150-260 days, about 150-250 days, about 150-240 days, about 150-230 days, about 150-220 days, about 150-210 days, about 160-200 days, or about 170-190 days after injection of the first dose. In certain embodiments, the second dose of the composition is injected about 150-330 days, about 150-330 days, about 150-330 days, about 105-330 days, about 140-330 days, about 150-330 days, about 160-330 days, about 170-330 days, or about 180-330 days after injection of the first dose. In some embodiments, the second dose of the composition is injected 150 or more days after injection of the first dose. In certain embodiments, the second dose of the composition is injected 150 days after injection of the first dose. In some embodiments, the second dose of the composition is injected about 180 days after injection of the first dose. In certain embodiments, the second dose of the composition is injected 180 days after injection of the first dose.

In some embodiments, the first dose comprises 100 μg to 200 μg, 110 μg to 190 μg, 120 μg to 180 μg, 130 μg to 170 μg, or 140 μg to 160 μg of the IL-10^{F129S} expression construct. In certain embodiments, the first dose comprises about 150 μg of the IL-10^{F129S} expression construct. In some embodiments, the first dose comprises 400 μg to 500 μg, 410 μg to 490 μg, 420 μg to 480 μg, 430 μg to 470 μg, or 440 μg to 460 μg of the IL-10^{F129S} expression construct. In certain embodiments, the first dose comprises about 450 μg of the IL-10^{F129S} expression construct. In some embodiments, the second dose comprises 400 μg to 500 μg, 410 μg to 490 μg, 420 μg to 480 μg, 430 μg to 470 μg, or 440 μg to 460 μg of the IL-10^{F129S} expression construct. In certain embodiments, the second dose comprises about 450 μg of the IL-10^{F129S} expression construct. In some embodiments, the first dose comprises about 150 μg of the IL-10^{F129S} expression construct, and the second dose comprises about 450 μg of the IL-10^{F129S} expression construct. In certain embodiments, the first dose comprises about 450 μg of the IL-10^{F129S} expression construct, and the second dose comprises about 450 μg of the IL-10^{F129S} expression construct. In some embodiments, the second dose of the composition is injected about 150-210 days after injection of the first dose.

In another aspect, provided herein is a method for treating an inflammatory joint disease in a subject, comprising: injecting into an inflamed joint of the subject a first dose of a composition comprising an IL-10^{F129S} expression construct, wherein the first dose of the composition comprises 450 μg of the IL-10^{F129S} expression construct; and injecting into the inflamed joint of the subject a second dose of the composition about 150-180 days after injection of the first dose, wherein the second dose of the composition comprises 450 μg of the IL-10^{F129S} expression construct. In another aspect, provided herein is a method for treating an inflammatory joint disease in a subject, comprising: injecting into an inflamed joint of the subject a first dose of a composition comprising an IL-10^{F129S} expression construct, wherein the first dose of the composition comprises 450 μg of the IL-10^{F129S} expression construct; and injecting into the inflamed joint of the subject a second dose of the composition about 180 days after injection of the first dose, wherein the second dose of the composition comprises 450 μg of the IL-10^{F129S} expression construct. In some embodiments, treating the subject with a first dose and a second dose results in increased efficacy compared to a method in which a subject is treated with only the first dose.

The methods described herein utilize a composition comprising an IL-10^{F129S} expression construct. In some embodiments, the construct used herein comprises: two AAV ITRs; a chicken β-actin promoter, a herpes simplex virus thymidine kinase (TK) promoter, an SV40 polyadenylation signal, and a cytomegalovirus immediate early promoter enhancer; and optionally, an antibiotic resistance marker. In some embodiments, the construct used herein comprises a sequence that is at least 80%, at least 85%, at least 90%, at least 95%, or at least 99% identical to the nucleotide sequence of SEQ ID NO: 1. In some embodiments, the expression construct consists of the nucleotide sequence of SEQ ID NO: 1.

As used herein, the composition injected into the joint comprises IL-10^{F129S} expression construct, as well as optional excipients and diluents. In some embodiments, the first dose of the composition and/or the second dose of the composition further comprises one or more excipients. In certain embodiments, the one or more excipients increase the uptake of the expression construct. In some embodiments, the one or more excipients are selected from the group consisting of a sugar, calcium phosphate, a dendrimer, an oligonucleotide, a high molecular weight hyaluronic acid, and a lipid. In some embodiments, the sugar is mannose, sucrose, or glucose. In certain embodiments, the sugar is D-mannose. In some embodiments, the sugar is sucrose. In certain embodiments, the one or more excipients are sugar and D-mannose. In some embodiments, the composition further comprises a diluent. In certain embodiments, the diluent is phosphate-buffered saline (pH 7.4). In some embodiments, the first and/or the second dose of the composition further comprises phosphate-buffered saline, sucrose, and D-mannose. In certain embodiments, the composition comprises about 3% w/v sucrose. In some embodiments, the composition comprises about 2.5 mg/mL D-mannose. In certain embodiments, the first and the second dose of the composition are formulated in a solution of phosphate-buffered saline (pH 7.4) with 3% w/v sucrose and 2.5 mg-mL D-mannose. In some embodiments, the composition comprises about 0.15-0.45 mg/mL of IL-10^{F129S} expression construct. In certain embodiments, the composition comprises about 0.15 mg/mL of IL-10^{F129S} expression construct. In some embodiments, the composition comprises about 0.45 mg/mL of IL-10^{F129S} expression construct.

As used herein, the composition comprising the IL-10^{F129S} expression construct is injected into a joint. In some embodiments, the inflamed joint is a knee, an elbow, a wrist, an ankle, a hip, a shoulder, a jaw, or a spine. In certain embodiments, the inflamed joint is a knee.

As detailed herein, the methods provided herein are methods for treating an inflammatory joint disease. In some embodiments, the inflammatory joint disease is arthritis, tendonitis, bursitis, inflammation of the ligament, synovitis, gout, facet syndrome, or systemic lupus erythematosus. In certain embodiments, the arthritis is rheumatoid arthritis. In some embodiments, the arthritis is osteoarthritis. In certain embodiments, the arthritis is osteoarthritis of the knee.

In some embodiments, the subject is a mammal. In certain embodiments, the subject is a human. In some embodiments, the subject is a primate, cattle, pig, horse, sheep, goat, camel, cat, or dog. In certain embodiments, the subject is an osteoarthritis patient with a WOMAC score of greater than 9. In certain embodiments, the subject is an osteoarthritis of the knee patient with a WOMAC score of greater than 9. In some embodiments, the subject is an osteoarthritis patient with a Kellgren-Lawrence (KL) grade of 1-4. In some embodiments, the subject benefits from treatment of the inflammatory joint disease for at least half a year from the injection of the first dose. In some embodiments, the composition administered in the first and the second dose result in no drug related serious adverse events. In some embodiments, the subject has minimal antibody production to IL-10 following injection of the composition for 360 days. In some embodiments, the subject has no anti-IL-10 antibodies within 360 days following injection of the composition.

Also provided herein is the use of a composition comprising an IL-10^{F129S} expression construct for the treatment of an inflammatory joint disease, wherein the composition is for administration in a first dose comprising 150 μg or 450 μg of the IL-10^{F129S} expression construct, and wherein the composition is for administration in a second dose comprising 450 μg of the IL-10^{F129S} expression construct 150-360 days after the first dose.

The present disclosure also provides a kit comprising a first dose and a second dose of an IL-10^{F129S} expression construct, wherein the first dose comprises 150 μg or 450 μg of the expression construct, and wherein the second dose comprises 450 μg of the expression construct; and wherein the first and/or the second dose of the composition is formulated in a sterile solution of phosphate-buffered saline (pH 7.4) with about 3% w/v sucrose and about 2.5 mg/mL D-mannose.

The present disclosure provides methods for treating an inflammatory joint disease in a subject, the method comprising injecting into an inflamed joint of the subject a first dose of a composition comprising an IL-10^{F129S} expression construct, wherein the first dose of the composition comprises 100 μg to 600 μg of the IL-10^{F129S} expression construct; and injecting into the inflamed joint of the subject a second dose of the composition 90-330 days after injection of the first dose, wherein the second dose of the composition comprises 100 μg to 600 μg of the IL-10^{F129S} expression construct. In certain embodiments, the IL-10^{F129S} expression construct is XT-150. Thus, in one aspect, the present disclosure provides methods for treating an inflammatory joint disease in a subject, the method comprising injecting into an inflamed joint of the subject a first dose of a composition comprising an IL-10^{F129S} expression construct, wherein the first dose of the composition comprises 100 μg to 600 μg of the IL-10^{F129S} expression construct; and injecting into the inflamed joint of the subject a second dose of the composition 150-210 days after injection of the first dose, wherein the second dose of the composition comprises 100 μg to 600 μg of the IL-10^{F129S} expression construct. In certain embodiments, the IL-10^{F129S} expression construct is XT-150.

In some embodiments, the method comprises injecting one or more additional doses (e.g., a third dose, a fourth dose, a fifth dose, etc.) into an inflamed joint of the subject. In some embodiments, the one or more additional doses are injected 90-330 days after injection of the previous dose. In some embodiments, the one or more additional doses are injected as needed (e.g., as symptoms of the inflammatory joint disease, such as pain, begin to reoccur in the subject). In some embodiments, the one or more additional doses are injected as needed, as determined by a medical professional. In some embodiments, additional doses are injected as needed for the life of the subject. In some embodiments, the one or more additional doses are injected before the reoccurrence of pain.

The second dose of the composition may be injected into an inflamed joint (e.g., knee, elbow, wrist, ankle, hip, shoulder, jaw, or spine) of the subject anywhere between 90 and 330 days after injection of the first dose. In some embodiments, the second dose of the composition is injected into the same inflamed joint as the first dose of the composition. In some embodiments, the second dose of the composition is injected anywhere between 90 and 270 days after injection of the first dose. In some embodiments, the second dose of the composition is injected about 100-260 days, about 110-250 days, about 120-240 days, about 130-230 days, about 140-220 days, about 150-210 days, about 160-200 days, or about 170-190 days after injection of the first dose. In some embodiments, the second dose of the composition is injected about 100-330 days, about 110-330 days, about 120-330 days, about 130-330 days, about 140-330 days, about 150-330 days, about 160-330 days, about 170-330 days, or about 180-330 days after injection of the first dose. In some embodiments, the second dose of the composition is injected about 90-120 days after injection of the first dose. The second dose of the composition may also be injected 180 or more days after injection of the first dose. In some embodiments, the second dose of the composition is injected about 180-330 days, about 180-320 days, about 180-310 days, about 180-300 days, about 180-290 days, about 180-280 days, about 180-270 days, about 180-260 days, about 180-250 days, about 180-240 days, about 180-230 days, about 180-220 days, about 180-210 days, about 180-200 days, or about 180-190 days after injection of the first dose. In certain embodiments, the second dose of the composition is injected about 170-190 days after injection of the first dose. In certain embodiments, the second dose of the composition is injected approximately 180 days after injection of the first dose. In certain embodiments, the second dose of the composition is injected at least 180 days after injection of the first dose. In certain embodiments, the second dose of the composition is injected at least 180 days after injection of the first dose but before 365 days after injection of the first dose. In certain embodiments, the second dose of the composition is injected 180 days after injection of the first dose.

The first dose and the second dose of the composition may each independently comprise about 100 μg to about 600 μg of the IL-10^{F129S} expression construct. In some embodiments, the first dose comprises 100 μg to 200 μg, 110 μg to 190 μg, 120 μg to 180 μg, 130 μg to 170 μg, or 140 μg to 160 μg of the IL-10^{F129S} expression construct (e.g., about 150 μg of the IL-10^{F129S} expression construct (e.g., XT-150)). In some embodiments, the first dose comprises 400 μg to 500 μg, 410 μg to 490 μg, 420 μg to 480 μg, 430 μg to 470 μg, or 440 μg to 460 μg of the IL-10^{F129S} expression construct (e.g., about 450 μg of the IL-10^{F129S} expression construct). In some embodiments, the second dose comprises 400 μg to 500 μg, 410 μg to 490 μg, 420 μg to 480 μg, 430 μg to 470 μg, or 440 μg to 460 μg of the IL-10^{F129S} expression construct (e.g., about 450 μg of the IL-10^{F129S} expression construct).

In certain embodiments, the present disclosure provides a method for treating an inflammatory joint disease in a subject comprising injecting into an inflamed joint of the subject a first dose of a composition comprising an IL-10F129S expression construct, wherein the first dose of the composition comprises 450 μg of the IL-10F129S expression construct; and injecting into the inflamed joint of the subject a second dose of the composition approximately 180 days after injection of the first dose, wherein the second dose of the composition comprises 450 μg of the IL-10F129S expression construct.

In certain embodiments, the present disclosure provides a method for treating an inflammatory joint disease in a subject comprising injecting into an inflamed joint of the subject a first dose of a composition comprising an IL-10^{F129S} expression construct, wherein the first dose of the composition comprises 450 μg of the IL-10^{F129S} expression construct; and injecting into the inflamed joint of the subject a second dose of the composition at least 180 days after injection of the first dose, wherein the second dose of the composition comprises 450 μg of the IL-10^{F129S} expression construct.

In certain embodiments, the first and/or the second dose of the composition further comprises phosphate-buffered saline, sucrose. and D-mannose. In some embodiments, the first and/or second dose of the administered composition is formulated in a solution of phosphate-buffered saline (pH 7.4) with sucrose and D-mannose. In some embodiments, the composition comprises about 3% w/v sucrose. In some embodiments, the composition comprises about 2.5 mg/mL D-mannose. In certain embodiments, the first and/or second dose of the composition is formulated in a solution of phosphate-buffered saline (pH 7.4) with 3% w/v sucrose and 2.5 mg/mL D-mannose.

In another aspect, the present disclosure provides for the use of a composition comprising an IL-10^{F129S} expression construct for the treatment of an inflammatory joint disease, wherein the composition is for administration in a first dose comprising 100 μg to 600 μg of the IL-10^{F129S} expression construct, and wherein the composition is for administration in a second dose comprising 100 μg to 600 μg of the IL-10^{F129S} expression construct 90-330 days after the first dose.

In another aspect, the present disclosure provides kits comprising a first dose and a second dose of an IL-10^{F129S} expression construct (e.g., XT-150). In some embodiments, the first dose comprises 100 μg to 600 μg of the expression construct, and the second dose comprises 100 μg to 600 μg of the expression construct. In certain embodiments, the first dose comprises about 450 μg of the expression construct. In certain embodiments, the second dose comprises about 450 μg of the expression construct. In some embodiments, the first and/or second dose of the composition is formulated in a sterile solution of phosphate-buffered saline (pH 7.4) with about 3% w/v sucrose and about 2.5 mg/mL D-mannose. The kits provided herein may be useful for treating a disease (e.g., an inflammatory joint disease) in a subject in need thereof.

### Methods for Treating an Inflammatory Joint Disease in a Subject

In one aspect, the present disclosure provides methods for treating an inflammatory joint disease in a subject, the method comprising injecting into an inflamed joint of the subject a first dose of a composition comprising an IL-10^{F129S} expression construct, wherein the first dose of the composition comprises 100 μg to 600 μg of the IL-10^{F129S} expression construct; and injecting into the inflamed joint of the subject a second dose of the composition 90-330 days after injection of the first dose, wherein the second dose of the composition comprises 100 μg to 600 μg of the IL-10^{F129S} expression construct.

In some embodiments, the second dose of the composition is injected into the same inflamed joint as the first dose of the composition. The second dose of the composition may be injected into an inflamed joint of the subject anywhere between 90 and 360 days after injection of the first dose. The second dose of the composition may be injected into an inflamed joint of the subject anywhere between 90 and 330 days after injection of the first dose. In some embodiments, the second dose of the composition is injected anywhere between 90 and 270 days after injection of the first dose. In some embodiments, the second dose of the composition is injected about 90-270 days, about 100-260 days, about 110-250 days, about 120-240 days, about 130-230 days, about 140-220 days, about 150-210 days, about 160-200 days, or about 170-190 days after injection of the first dose. In some embodiments, the second dose of the composition is injected about 100-260 days, about 110-200 days, about 120-190 days, about 120-180 days, about 130-190 days, about 130-180 days, about 140-190 days, about 140-180 days, about 150-190 days, about 150-180 days, about 160-190 days, about 160-180 days, about 170-190 days, or about 170-180 days after injection of the first dose. In some embodiments, the second dose of the composition is injected about 100-330 days, about 110-330 days, about 120-330 days, about 130-330 days, about 140-330 days, about 150-330 days, about 160-330 days, about 170-330 days, or about 180-330 days after injection of the first dose. In some embodiments, the second dose of the composition is injected about 150-270 days, about 150-260 days, about 150-250 days, about 150-240 days, about 150-230 days, about 150-220 days, about 150-210 days, about 160-200 days, or about 170-190 days after injection of the first dose. In some embodiments, the second dose of the composition is injected about 150-270 days, about 150-260 days, about 150-250 days, about 150-240 days, about 150-230 days, about 150-220 days, about 150-210 days, about 150-200 days, or about 150-190 days after injection of the first dose. In some embodiments, the second dose of the composition is injected about 100-260 days, about 110-250 days, about 120-240 days, about 130-230 days, about 140-220 days, about 150-210 days, about 160-200 days, or about 170-190 days after injection of the first dose. In some embodiments, the second dose of the composition is injected about 100-330 days, about 110-330 days, about 120-330 days, about 130-330 days, about 140-330 days, about 150-330 days, about 160-330 days, about 170-330 days, or about 180-330 days after injection of the first dose.

In some embodiments, the second dose of the composition is injected 180 or more days after injection of the first dose. In some embodiments, the second dose of the composition is injected about 180-330 days, about 180-320 days, about 180-310 days, about 180-300 days, about 180-290 days, about 180-280 days, about 180-270 days, about 180-260 days, about 180-250 days, about 180-240 days, about 180-230 days, about 180-220 days, about 180-210 days, about 180-200 days, or about 180-190 days after injection of the first dose.

In some embodiments, the second dose of the composition is injected 150 or more days after injection of the first dose. In some embodiments, the second dose of the composition is injected about 150-330 days, about 150-320 days, about 150-310 days, about 150-300 days, about 150-290 days, about 150-280 days, about 150-270 days, about 150-260 days, about 150-250 days, about 150-240 days, about 150-230 days, about 150-220 days, about 150-210 days, about 150-200 days, or about 150-190 days after injection of the first dose. In some embodiments, the second dose of the composition is injected about 150-330 days, about 150-330 days, about 150-330 days, about 105-330 days, about 140-330 days, about 150-330 days, about 160-330 days, about 170-330 days, or about 180-330 days after injection of the first dose.

In some embodiments, the second dose of the composition is injected about 90-120 days after injection of the first dose. In some embodiments, the second dose of the composition is injected about 90-120 days after injection of the first dose. In some embodiments, the second dose of the composition is injected about 150-180 days after injection of the first dose. The second dose of the composition may also be injected 130 or more days after injection of the first dose. The second dose of the composition may also be injected 135 or more days after injection of the first dose. The second dose of the composition may also be injected 14—or more days after injection of the first dose. The second dose of the composition may also be injected 145 or more days after injection of the first dose. The second dose of the composition may also be injected 150 or more days after injection of the first dose. The second dose of the composition may also be injected 155 or more days after injection of the first dose. The second dose of the composition may also be injected 160 or more days after injection of the first dose. The second dose of the composition may also be injected 165 or more days after injection of the first dose. The second dose of the composition may also be injected 170 or more days after injection of the first dose. The second dose of the composition may also be injected 175 or more days after injection of the first dose. The second dose of the composition may also be injected 180 or more days after injection of the first dose. The second dose of the composition may also be injected about 180 or more days after injection of the first dose. The second dose of the composition may also be injected 180 or more days after injection of the first dose. The second dose of the composition may also be injected 185 or more days after injection of the first dose. The second dose of the composition may also be injected 190 or more days after injection of the first dose. The second dose of the composition may also be injected 195 or more days after injection of the first dose. The second dose of the composition may also be injected 200 or more days after injection of the first dose. In some embodiments, the second dose of the composition is injected about 180-330 days, about 180-320 days, about 180-310 days, about 180-300 days, about 180-290 days, about 180-280 days, about 180-270 days, about 180-260 days, about 180-250 days, about 180-240 days, about 180-230 days, about 180-220 days, about 180-210 days, about 180-200 days, or about 180-190 days after injection of the first dose. In certain embodiments, the second dose of the composition is injected about 170-190 days after injection of the first dose.

In some embodiments, the second dose of the composition is injected about 15-330 days after injection of the first dose. In some embodiments, the second dose of the composition is injected about 20-330 days after injection of the first dose. In some embodiments, the second dose of the composition is injected about 30-330 days after injection of the first dose. In some embodiments, the second dose of the composition is injected about 40-330 days after injection of the first dose. In some embodiments, the second dose of the composition is injected about 50-330 days after injection of the first dose. In some embodiments, the second dose of the composition is injected about 60-330 days after injection of the first dose. In some embodiments, the second dose of the composition is injected about 90-330 days after injection of the first dose. In some embodiments, the second dose of the composition is injected about 120-330 days after injection of the first dose. In some embodiments, the second dose of the composition is injected about 15-150 days after injection of the first dose. In some embodiments, the second dose of the composition is injected about 20-120 days after injection of the first dose. In some embodiments, the second dose of the composition is injected about 30-90 days after injection of the first dose. In some embodiments, the second dose of the composition is injected about 40-60 days after injection of the first dose. In some embodiments, the second dose of the composition is injected about 40-50 days after injection of the first dose. In some embodiments, the second dose of the composition is injected about 20-60 days after injection of the first dose. In some embodiments, the second dose of the composition is injected about 30-40 days after injection of the first dose. In some embodiments, the second dose of the composition is injected about 20-40 days after injection of the first dose.

In certain embodiments, the second dose of the composition is injected approximately 180 days after injection of the first dose. In certain embodiments, the second dose of the composition is injected at least 180 days after injection of the first dose. In certain embodiments, the second dose of the composition is injected at least 180 days after injection of the first dose but before 365 days after injection of the first dose. In certain embodiments, the second dose of the composition is injected 180 days after injection of the first dose.

In certain embodiments, the second dose of the composition is injected approximately 150 days after injection of the first dose. In some embodiments, the second dose of the composition is injected 150 or more days after injection of the first dose. In certain embodiments, the second dose of the composition is injected at least 150 days after injection of the first dose. In certain embodiments, the second dose of the composition is injected at least 150 days after injection of the first dose but before 365 days after injection of the first dose. In certain embodiments, the second dose of the composition is injected 150 days after injection of the first dose.

In some embodiments, the method comprises injecting one or more additional doses (e.g., a third dose, a fourth dose, a fifth dose, etc.) into an inflamed joint of the subject. In some embodiments, the one or more additional doses are injected about 15-330 days after injection of the previous dose. In some embodiments, the one or more additional doses are injected about 20-330 days after injection of the previous dose. In some embodiments, the one or more additional doses are injected about 30-330 days after injection of the previous dose. In some embodiments, the one or more additional doses are injected about 40-330 days after injection of the previous dose. In some embodiments, the one or more additional doses are injected about 50-330 days after injection of the previous dose. In some embodiments, the one or more additional doses are injected about 60-330 days after injection of the previous dose. In some embodiments, the one or more additional doses are injected about 90-330 days after injection of the previous dose. In some embodiments, the one or more additional doses are injected about 120-330 days after injection of the previous dose. In some embodiments, the one or more additional doses are injected as needed (e.g., as symptoms of the inflammatory joint disease, such as pain, begin to reoccur in the subject). In some embodiments, the one or more additional doses are injected as needed, as determined by a medical professional. In some embodiments, additional doses are injected as needed for the life of the subject. In some embodiments, the one or more additional doses are injected before the reoccurrence of pain.

The first dose and the second dose of the composition may each independently comprise about 25 μg to 750 μg of the IL-10^{F129S} expression construct. The first dose and the second dose of the composition may each independently comprise about 50 μg to 500 μg of the IL-10^{F129S} expression construct. The first dose and the second dose of the composition may each independently comprise about 50 μg to 50 μg of the IL-10^{F129S} expression construct. The first dose and the second dose of the composition may each independently comprise about 100 μg to 600 μg of the IL-10^{F129S} expression construct. In some embodiments, the first dose comprises 100 μg to 200 μg, 110 μg to 190 μg, 120 μg to 180 μg, 130 μg to 170 μg, or 140 μg to 160 μg of the IL-10^{F129S} expression construct (e.g., about 150 μg of the IL-10^{F129S} expression construct). In some embodiments, the first dose comprises 400 μg to 500 μg, 410 μg to 490 μg, 420 μg to 480 μg, 430 μg to 470 μg, or 440 μg to 460 μg of the IL-10^{F129S} expression construct (e.g., about 450 μg of the IL-10^{F129S} expression construct). In some embodiments, the second dose comprises 400 μg to 500 μg, 410 μg to 490 μg, 420 μg to 480 μg, 430 μg to 470 μg, or 440 μg to 460 μg of the IL-10^{F129S} expression construct (e.g., about 450 μg of the IL-10^{F129S} expression construct). In some embodiments, the first dose comprises about 150 μg of the IL-10^{F129S} expression construct. In some embodiments, the first dose comprises about 450 μg of the IL-10^{F129S} expression construct. In some embodiments, the second dose comprises about 450 μg of the IL-10^{F129S} expression construct.

Any combination of the ranges described above for the first dose and the second dose may be utilized in the methods described herein. In some embodiments, the first dose comprises about 150 μg of the IL-10^{F129S} expression construct, and the second dose comprises about 450 μg of the IL-10^{F129S} expression construct. In some embodiments, the first dose comprises about 450 μg of the IL-10^{F129S} expression construct, and the second dose comprises about 450 μg of the IL-10^{F129S} expression construct. In some embodiments, the first dose comprises about 150 μg of the IL-10^{F129S} expression construct, and the second dose comprises about 450 μg of the IL-10^{F129S} expression construct. In some embodiments, the first dose comprises about 450 μg of the IL-10^{F129S} expression construct, and the second dose comprises about 450 μg of the IL-10^{F129S} expression construct. In certain embodiments, the first dose comprises about 450 μg of the IL-10^{F129S} expression construct, the second dose comprises about 450 μg of the IL-10^{F129S} expression construct, and the second dose of the composition is injected 180 days after injection of the first dose.

In certain embodiments, the first dose comprises about 450 μg of the IL-10^{F129S} expression construct, the second dose comprises about 450 μg of the IL-10^{F129S} expression construct, and the second dose of the composition is injected 150-360 days after injection of the first dose. In certain embodiments, the first dose comprises about 450 μg of the IL-10^{F129S} expression construct, the second dose comprises about 450 μg of the IL-10^{F129S} expression construct, and the second dose of the composition is injected 150-274 days after injection of the first dose. In certain embodiments, the first dose comprises about 450 μg of the IL-10^{F129S} expression construct, the second dose comprises about 450 μg of the IL-10^{F129S} expression construct, and the second dose of the composition is injected 150-240 days after injection of the first dose. In certain embodiments, the first dose comprises about 450 μg of the IL-10^{F129S} expression construct, the second dose comprises about 450 μg of the IL-10^{F129S} expression construct, and the second dose of the composition is injected 150-210 days after injection of the first dose. In certain embodiments, the first dose comprises about 450 μg of the IL-10^{F129S} expression construct, the second dose comprises about 450 μg of the IL-10^{F129S} expression construct, and the second dose of the composition is injected 150-210 days after injection of the first dose.

In certain embodiments, the first dose comprises about 450 μg of the IL-10^{F129S} expression construct, the second dose comprises about 450 μg of the IL-10^{F129S} expression construct, and the second dose of the composition is injected 180 days after injection of the first dose. In certain embodiments, the first dose comprises about 450 μg of the IL-10^{F129S} expression construct, the second dose comprises about 450 μg of the IL-10^{F129S} expression construct, and the second dose of the composition is injected 165 days after injection of the first dose. In certain embodiments, the first dose comprises about 450 μg of the IL-10^{F129S} expression construct, the second dose comprises about 450 μg of the IL-10^{F129S} expression construct, and the second dose of the composition is injected 150 days after injection of the first dose.

In certain embodiments, the first dose comprises about 450 μg of the IL-10^{F129S} expression construct, the second dose comprises about 450 μg of the IL-10^{F129S} expression construct, and the second dose of the composition is injected 200 days after injection of the first dose. In certain embodiments, the first dose comprises about 450 μg of the IL-10^{F129S} expression construct, the second dose comprises about 450 μg of the IL-10^{F129S} expression construct, and the second dose of the composition is injected 240 days after injection of the first dose. In certain embodiments, the first dose comprises about 450 μg of the IL-10^{F129S} expression construct, the second dose comprises about 450 μg of the IL-10^{F129S} expression construct, and the second dose of the composition is injected 270 days after injection of the first dose.

The IL-10^{F129S} expression construct may comprise any vector or expression platform, as well as any of the nucleic acid elements (e.g., promoters, polyadenylation signals, marker genes, etc.) described herein. In some embodiments, the IL-10^{F129S} expression construct comprises a plasmid backbone, such as a bacterial or viral plasmid backbone. In some embodiments, the IL-10^{F129S} expression construct comprises a nucleic acid sequence encoding IL-10^{F129S}. In some embodiments, the IL-10^{F129S} expression construct comprises adeno-associated virus (AAV) inverted terminal repeat (ITR) sequences flanking the nucleic acid sequence encoding interleukin-10^{F129S} (i.e., at the 5′ and 3′ positions relative to the nucleic acid sequence encoding IL-10^{F129S}).

In some embodiments, the IL-10^{F129S} expression construct comprises one or more promoter sequences. Exemplary promoters that may be used in the expression constructs utilized in the methods described herein include, but are not limited to, chicken or human β-actin promoters, cytomegalovirus immediate early promoters, glyceraldehyde 3-phosphate dehydrogenase (GADPH) promoters, elongation factor 1α (eF1α) promoters, GFAP promoters, murine leukemia virus (MLV) promoters, herpes simplex virus thymidine kinase (TK) promoters, and woodchuck hepatitis virus post-transcriptional regulatory element (WPRE) promoters. In some embodiments, the promoter (e.g., constitutive promoter) is selected from the group consisting of ubiquitin promoters, CMV promoters, β-actin promoters, histone H4 promoters, EF-1α promoters, PGK gene promoters, promoter elements controlled by RNA polymerase II, promoter elements controlled by RNA polymerase I, promoter elements controlled by RNA polymerase III, U6 promoters (e.g., U6-1 promoters, U6-8 promoters, U6-9 promoters), H1 promoter, 7SL promoter, human Y promoters (e.g., hY1 promoters, hY3 promoters, hY4 promoters, and hY5 promoters), human MRP-7-2 promoter, adenovirus VA1 promoter, human tRNA promoters, and 5s ribosomal RNA promoters. Exemplary promoters that may be used in the expression constructs utilized in the methods described herein include, but are not limited to, chicken or human β-actin promoters, cytomegalovirus immediate early promoters, CAG promoters, glyceraldehyde 3-phosphate dehydrogenase (GADPH) promoters, elongation factor 1α (eF1α) promoters, GFAP promoters, murine leukemia virus (MLV) promoters, herpes simplex virus thymidine kinase (TK) promoters, and woodchuck hepatitis virus post-transcriptional regulatory element (WPRE) promoters. In some embodiments, the promoter (e.g., constitutive promoter) is selected from the group consisting of ubiquitin promoters, CMV promoters, β-actin promoters, CAG promoters, histone H4 promoters, EF-1α promoters, PGK gene promoters, promoter elements controlled by RNA polymerase II, promoter elements controlled by RNA polymerase I, promoter elements controlled by RNA polymerase III, U6 promoters (e.g., U6-1 promoters, U6-8 promoters, U6-9 promoters), H1 promoter, 7SL promoter, human Y promoters (e.g., hY1 promoters, hY3 promoters, hY4 promoters, and hY5 promoters), human MRP-7-2 promoter, adenovirus VA1 promoter, human tRNA promoters, and 5s ribosomal RNA promoters. Any promoter known in the art may be used in the expression constructs described herein, and skilled artisan would be capable of identifying additional promoters suitable for use in the presently described methods. In certain embodiments, the IL-10^{F129S} expression construct comprises a cytomegalovirus immediate early promoter. In certain embodiments, the IL-10^{F129S} expression construct comprises a cytomegalovirus immediate early promoter enhancer. In certain embodiments, the IL-10^{F129S} expression construct comprises a chicken β-actin promoter. In certain embodiments, the IL-10^{F129S} expression construct comprises a herpes simplex virus thymidine kinase (TK) promoter. In certain embodiments, the IL-10^{F129S} expression construct comprises a CAG promoter. In certain embodiments, the IL-10^{F129S} expression construct comprises a eF1α promoter.

In some embodiments, the IL-10^{F129S} expression construct comprises a polyadenylation signal operably linked to the nucleic acid sequence encoding IL-10^{F129S}. The polyadenylation signal may be selected from the group consisting of an SV40 polyadenylation signal, a bovine growth hormone polyadenylation signal, and a synthetic polyadenylation signal. In certain embodiments, the polyadenylation signal is an SV40 polyadenylation signal.

In some embodiments, the IL-10^{F129S} expression construct comprises a marker gene. The marker gene may be, for example, an antibiotic resistance gene (e.g., a neomycin resistance marker, an ampicillin resistance marker, or a kanamycin resistance marker). In certain embodiments, the marker gene is a kanamycin resistance marker.

The IL-10^{F129S} expression construct may also comprise one or more nuclear targeting sequences. A nuclear targeting sequence may be included, for example, 5′ to the nucleic acid sequence encoding interleukin-10^{F129S} on the expression construct, or 3′ to the nucleic acid sequence encoding interleukin-10^{F129S}. In some embodiments, the IL-10^{F129S} expression construct comprises one or more nuclear targeting sequences 5′ to the nucleic acid sequence encoding interleukin-10^{F129S} and one or more nuclear targeting sequences 3′ to the nucleic acid sequence encoding interleukin-10^{F129S}. In some embodiments, a nuclear targeting sequence is an inverted terminal repeat (an ITR, e.g., an adeno-associated virus (AAV) ITR).

The IL-10^{F129S} expression construct may also comprise one or more control sequences operably linked to the nucleic acid sequence encoding IL-10^{F129S}. The one or more control sequences may each be independently selected from the group consisting of transcription termination sequences, upstream regulatory domains, and internal ribosome entry sites.

In some embodiments, the IL-10^{F129S} expression construct comprises two AAV ITRs; a chicken β-actin promoter, a herpes simplex virus thymidine kinase (TK) promoter, an SV40 polyadenylation signal, and a cytomegalovirus immediate early promoter enhancer; and optionally, an antibiotic resistance gene. In some embodiments, the IL-10^{F129S} expression construct comprises two AAV ITRs; a cytomegalovirus immediate early promoter, an SV40 polyadenylation signal, and a cytomegalovirus immediate early promoter enhancer; and optionally, an antibiotic resistance gene. In some embodiments, the IL-10^{F129S} expression construct comprises two AAV ITRs; a CAG promoter, a herpes simplex virus thymidine kinase (TK) promoter, an SV40 polyadenylation signal, and a cytomegalovirus immediate early promoter enhancer; and optionally, an antibiotic resistance gene. In some embodiments, the IL-10^{F129S} expression construct comprises two AAV ITRs; a eF1α promoter, a herpes simplex virus thymidine kinase (TK) promoter, an SV40 polyadenylation signal, and a cytomegalovirus immediate early promoter enhancer; and optionally, an antibiotic resistance gene.

In some embodiments, the IL-10^{F129S} expression construct comprises two AAV ITRs; a chicken β-actin promoter, a herpes simplex virus thymidine kinase (TK) promoter, an SV40 polyadenylation signal, and a cytomegalovirus immediate early promoter enhancer; and a neomycin resistance marker. The IL-10^{F129S} expression construct may also comprise two AAV ITRs; a cytomegalovirus immediate early promoter, an SV40 polyadenylation signal, and a cytomegalovirus immediate early promoter enhancer; and an ampicillin resistance marker. In some embodiments, the IL-10^{F129S} expression construct comprises two AAV ITRs; a cytomegalovirus immediate early promoter, an SV40 polyadenylation signal, and a cytomegalovirus immediate early promoter enhancer; and a kanamycin resistance marker.

In certain embodiments, the expression construct comprises a circular vector (i.e., the XT-150 plasmid) comprising a sequence that is at least 80%, at least 85%, at least 90%, at least 95%, at least 98%, or at least 99% identical to the nucleotide sequence:

(SEQ ID NO: 1)
AGCTTTGCAGCCGGGCGTAATAGCGAAGAGGCCCGCACCGATCGCCCTTCCCAACA
GTTGCGCAGCCTGAATGGCGAATGGACGCGCCCTGTAGCGGCGCATTAAGCGCGGC
GGGTGTGGTGGTTACGCGCAGCGTGACCGCTACACTTGCCAGCGCCCTAGCGCCCGC
TCCTTTCGCTTTCTTCCCTTCCTTTCTCGCCACGTTCGCCGGCTTTCCCCGTCAAGCTC
TAAATCGGGGGCTCCCTTTAGGGTTCCGATTTAGTGCTTTACGGCACCTCGACCCCA
AAAAACTTGATTAGGGTGATGGTTCACGTAGTGGGCCATCGCCCTGATAGACGGTTT
TTCGCCCTTTGACGTTGGAGTCCACGTTCTTTAATAGTGGACTCTTGTTCCAAACTGG
AACAACACTCAACCCTATCTCGGTCTATTCTTTTGATTTATAAGGGATTTTGCCGATT
TCGGCCTATTGGTTAAAAAATGAGCTGATTTAACAAAAATTTAACGCGAATTTTAAC
AAAATATTAACGCTTACAATTTCCTGATGCGGTATTTTCTCCTTACGCATCTGTGCGG
TATTTCACACCGCATATGGTGCACTCTCAGTACAATCTGCTCTGATGCCGCATAGTTA
AGCCAGCCCCGACACCCGCCAACACCCGCTGACGCGCCCTGACGGGCTTGTCTGCTC
CCGGCATCCGCTTACAGACAAGCTGTGACCGTCTCCGGGAGCTGCATGTGTCAGAG
GTTTTCACCGTCATCACCGAAACGCGCGAGACGAAAGGGCCTCGTGATACGCCTATT
TTTATAGGTTAATGTCATGATAATAATGGTTTCTTAGACGTCAGGTGGCACTTTTCGG
GGAAATGTGCGCGGAACCCCTATTTGTTTATTTTTCTAAATACATTCAAATATGTATC
CGCTCATGAGACAATAACCCTGATAAATGCTTCAATAATATTGAAAAAGGAAGAGT
AATTGAAACAAGATGGATTGCACGCAGGTTCTCCGGCCGCTTGGGTGGAGAGGCTA
TTCGGCTATGACTGGGCACAACAGACAATCGGCTGCTCTGATGCCGCCGTGTTCCGG
CTGTCAGCGCAGGGGCGCCCGGTTCTTTTTGTCAAGACCGACCTGTCCGGTGCCCTG
AATGAACTGCAGGACGAGGCAGCGCGGCTATCGTGGCTGGCCACGACGGGCGTTCC
TTGCGCAGCTGTGCTCGACGTTGTCACTGAAGCGGGAAGGGACTGGCTGCTATTGGG
CGAAGTGCCGGGGCAGGATCTCCTGTCATCTCACCTTGCTCCTGCCGAGAAAGTATC
CATCATGGCTGATGCAATGCGGCGGCTGCATACGCTTGATCCGGCTACCTGCCCATT
CGACCACCAAGCGAAACATCGCATCGAGCGAGCACGTACTCGGATGGAAGCCGGTC
TTGTCGATCAGGATGATCTGGACGAAGAGCATCAGGGGCTCGCGCCAGCCGAACTG
TTCGCCAGGCTCAAGGCGCGCATGCCCGACGGCGAGGATCTCGTCGTGACCCATGG
CGATGCCTGCTTGCCGAATATCATGGTGGAAAATGGCCGCTTTTCTGGATTCATCGA
CTGTGGCCGGCTGGGTGTGGCGGACCGCTATCAGGACATAGCGTTGGCTACCCGTGA
TATTGCTGAAGAGCTTGGCGGCGAATGGGCTGACCGCTTCCTCGTGCTTTACGGTAT
CGCCGCTCCCGATTCGCAGCGCATCGCCTTCTATCGCCTTCTTGACGAGTTCTTCTGA
GTAACCGTCAGACCAAGTTTACTCATATATACTTTAGATTGATTTAAAACTTCATTTT
TAATTTAAAAGGATCTAGGTGAAGATCCTTTTTGATAATCTCATGACCAAAATCCCT
TAACGTGAGTTTTCGTTCCACTGAGCGTCAGACCCCGTAGAAAAGATCAAAGGATCT
TCTTGAGATCCTTTTTTTCTGCGCGTAATCTGCTGCTTGCAAACAAAAAAACCACCGC
TACCAGCGGTGGTTTGTTTGCCGGATCAAGAGCTACCAACTCTTTTTCCGAAGGTAA
CTGGCTTCAGCAGAGCGCAGATACCAAATACTGTTCTTCTAGTGTAGCCGTAGTTAG
GCCACCACTTCAAGAACTCTGTAGCACCGCCTACATACCTCGCTCTGCTAATCCTGT
TACCAGTGGCTGCTGCCAGTGGCGATAAGTCGTGTCTTACCGGGTTGGACTCAAGAC
GATAGTTACCGGATAAGGCGCAGCGGTCGGGCTGAACGGGGGGTTCGTGCACACAG
CCCAGCTTGGAGCGAACGACCTACACCGAACTGAGATACCTACAGCGTGAGCTATG
AGAAAGCGCCACGCTTCCCGAAGGGAGAAAGGCGGACAGGTATCCGGTAAGCGGC
AGGGTCGGAACAGGAGAGCGCACGAGGGAGCTTCCAGGGGGAAACGCCTGGTATCT
TTATAGTCCTGTCGGGTTTCGCCACCTCTGACTTGAGCGTCGATTTTTGTGATGCTCG
TCAGGGGGGCGGAGCCTATGGAAAAACGCCAGCAACGCGGCCTTTTTACGGTTCCT
GGCCTTTTGCTGGCCTTTTGCTCACATGTTCTTTCCTGCGTTATCCCCTGATTCTGTGG
ATAACCGTATTACCGCCTTTGAGTGAGCTGATACCGCTCGCCGCAGCCGAACGACCG
AGCGCAGCGAGTCAGTGAGCGAGGAAGCGGAAGAGCGCCCAATACGCAAACCGCC
TCTCCCCGCGCGTTGGCCGATTCATTAATGCAGGGCTGCAGCATGCCTGCAGGCAGC
TGCGCGCTCGCTCGCTCACTGAGGCCGCCCGGGCGTCGGGCGACCTTTGGTCGCCCG
GCCTCAGTGAGCGAGCGAGCGCGCAGAGAGGGAGTGGCCAACTCCATCACTAGGGG
TTCCTGCGGCCGCACGCGTATCAATATTGGCCATTAGCCATATTATTCATTGGTTATA
TAGCATAAATCAATATTGGCTATTGGCCATTGCATACGTTGTATCTATATCATAATAT
GTACATTTATATTGGCTCATGTCCAATATGACCGCCATGTTGGCATTGATTATTGACT
AGTTATTAATAGTAATCAATTACGGGGTCATTAGTTCATAGCCCATATATGGAGTTC
CGCGTTACATAACTTACGGTAAATGGCCCGCCTGGCTGACCGCCCAACGACCCCCGC
CCATTGACGTCAATAATGACGTATGTTCCCATAGTAACGCCAATAGGGACTTTCCAT
TGACGTCAATGGGTGGAGTATTTACGGTAAACTGCCCACTTGGCAGTACATCAAGTG
TATCATATGCCAAGTCCGCCCCCTATTGACGTCAATGACGGTAAATGGCCCGCCTGG
CATTATGCCCAGTACATGACCTTACGGGACTTTCCTACTTGGCAGTACATCTACGTAT
TAGTCATCGCTATTACCATGGTCGAGGTGAGCCCCACGTTCTGCTTCACTCTCCCCAT
CTCCCCCCCCTCCCCACCCCCAATTTTGTATTTATTTATTTTTTAATTATTTTGTGCAG
CGATGGGGGCGGGGGGGGGGGGGGGGCGCGCGCCAGGCGGGGGGGGGCGGGGCG
AGGGGCGGGGCGGGGCGAGGCGGAGAGGTGCGGCGGCAGCCAATCAGAGCGGCGC
GCTCCGAAAGTTTCCTTTTATGGCGAGGCGGCGGCGGCGGCGGCCCTATAAAAAGC
GAAGCGCGCGGCGGGCGGGAGTCGCTGCGCGCTGCCTTCGCCCCGTGCCCCGCTCC
GCCGCCGCCTCGCGCCGCCCGCCCCGGCTCTGACTGACCGCGTTACTCCCACAGGTG
AGCGGGCGGGACGGCCCTTCTCCTCCGGGCTGTAATTAGCGCTTGGTTTAATGACGG
CTTGTTTCTTTTCTGTGGCTGCGTGAAAGCCTTGAGGGGCTCCGGGAGGGCCCTTTGT
GCGGGGGGAGCGGCTCGGGGGGTGCGTGCGTGTGTGTGTGCGTGGGGAGCGCCGCG
TGCGGCTCCGCGCTGCCCGGCGGCTGTGAGCGCTGCGGGCGCGGCGCGGGGCTTTG
TGCGCTCCGCAGTGTGCGCGAGGGGAGCGCGGCCGGGGGCGGTGCCCCGCGGTGCG
GGGGGGGCTGCGAGGGGAACAAAGGCTGCGTGCGGGGTGTGTGCGTGGGGGGGTG
AGCAGGGGGTGTGGGCGCGTCGGTCGGGCTGCAACCCCCCCTGCACCCCCCTCCCC
GAGTTGCTGAGCACGGCCCGGCTTCGGGTGCGGGGCTCCGTACGGGGCGTGGCGCG
GGGCTCGCCGTGCCGGGCGGGGGGTGGCGGCAGGTGGGGGTGCCGGGCGGGGCGG
GGCCGCCTCGGGCCGGGGAGGGCTCGGGGGAGGGGCGCGGCGGCCCCCGGAGCGC
CGGCGGCTGTCGAGGCGCGGCGAGCCGCAGCCATTGCCTTTTATGGTAATCGTGCGA
GAGGGCGCAGGGACTTCCTTTGTCCCAAATCTGTGCGGAGCCGAAATCTGGGAGGC
GCCGCCGCACCCCCTCTAGCGGGCGCGGGGCGAAGCGGTGCGGCGCCGGCAGGAAG
GAAATGGGCGGGGAGGGCCTTCGTGCGTCGCCGCGCCGCCGTCCCCTTCTCCCTCTC
CAGCCTCGGGGCTGTCCGCGGGGGGACGGCTGCCTTCGGGGGGGACGGGGCAGGGC
GGGGTTCGGCTTCTGGCGTGTGACCGGCGGCTCTAGAGCCTCTGCTAACCATGTTCA
TGCCTTCTTCTTTTTCCTACAGCTCCTGGGCAACGTGCTGGTTATTGTGCTGTCTCATC
ATTTTGGCAAAGAATTCGATATCAAGCTTGGTACCGAGCTCGGATCCACTAGTAACG
GCCGCCAGTGTGCTGGAATTCGCCCTTCCGCAGCGCCAGCATGCACAGCTCAGCACT
GCTCTGTTGCCTGGTCCTCCTGACTGGGGTGAGGGCCAGCCCAGGCCAGGGCACCCA
GTCTGAGAACAGCTGCACCCACTTCCCAGGCAACCTGCCTAACATGCTTCGAGATCT
CCGAGATGCCTTCAGCAGAGTGAAGACTTTCTTTCAAATGAAGGATCAGCTGGACA
ACTTGTTGTTAAAGGAGTCCTTGCTGGAGGACTTTAAGGGTTACCTGGGTTGCCAAG
CCTTGTCTGAGATGATCCAGTTTTACCTGGAGGAGGTGATGCCCCAAGCTGAGAACC
AAGACCCAGACATCAAGGCGCATGTGAACTCCCTGGGGGAGAACCTGAAGACCCTC
AGGCTGAGGCTACGGCGCTGTCATCGATCTCTTCCCTGTGAAAACAAGAGCAAGGC
CGTGGAGCAGGTGAAGAATGCCTTTAATAAGCTCCAAGAGAAAGGCATCTACAAAG
CCATGAGTGAGTTTGACATCTTCATCAACTACATAGAAGCCTACATGACAATGAAGA
TACGAAACTGAGACATCAGGGTGGCAAGCCGAATTCCCCCTAGCGGAAGGGCGAAT
TCTGCAGAATCGGATCCATCGATACCGTCGACCTCGAGGGGGGGCCCGGTACCCAA
TTCGCCCTATAGTGAGTCGTATTACGCGCGCAGCGGCCGACCATGGCCCAACTTGTT
TATTGCAGCTTATAATGGTTACAAATAAAGCAATAGCATCACAAATTTCACAAATAA
AGCATTTTTTTCACTGCATTCTAGTTGTGGTTTGTCCAAACTCATCAATGTATCTTAT
CATGTCTGGATCTCCGGACACGTGCGGACCGAGCGGCCGCAGGAACCCCTAGTGAT
GGAGTTGGCCACTCCCTCTCTGCGCGCTCGCTCGCTCACTGAGGCCGGGCGACCAAA
GGTCGCCCGACGCCCGGGCTTTGCCCGGGCGGCCTCAGTGAGCGAGCGAGCGCGCA
GCTGCCTGCAGGTCTGAGACAATAACCCTGATAAATGCTTCAATAATGTA.

In some embodiments, the expression construct comprises of the nucleotide sequence of SEQ ID NO: 1. In certain embodiments, the expression construct consists of the nucleotide sequence of SEQ ID NO: 1. In certain embodiments, the expression construct consists of the nucleotide sequence of SEQ ID NO: 1. In certain embodiments, the mutation comprises one or more deletions. In some embodiments, the mutation comprises one or more insertions. In certain embodiments, the mutation comprises insertions and deletions. In some embodiments, the mutation comprises one or more insertions and deletions. In some embodiments, the mutation comprises a point mutation with a single amino acid has been replaced (i.e., deleted then inserted). In certain embodiments, the vector comprises one or more, two or more, three or more, four or more, five or more, six or more, seven or more, eight or more, nine or more, or ten or more mutations relative to the nucleotide sequence of SEQ ID NO: 1.

In some embodiments, the vector comprises a nucleic acid sequence encoding IL-10, a nucleic acid sequence encoding IL-10R1, a plasmid backbone, CMV promoter, β-globin intron, growth hormone polyA, and 2 AAV2 ITRs. In some embodiments, the vector comprises a nucleic acid sequence encoding IL-10, a nucleic acid sequence encoding IL-10R1, a plasmid backbone, CAG promoter, β-globin intron, growth hormone polyA, and 2 AAV2 ITRs. In some embodiments, the vector comprises a nucleic acid sequence encoding IL-10, a nucleic acid sequence encoding IL-10R1, a plasmid backbone, eF1α promoter, β-globin intron, SV40 polyA, and 2 AAV2 ITRs.

In certain embodiments, the expression construct comprises a circular vector (i.e., the XT-151 plasmid) comprising a sequence that is at least 80%, at least 85%, at least 90%, at least 95%, at least 98%, or at least 99% identical to the nucleotide sequence:

(SEQ ID NO: 2)
AGCTTTGCAGCCG
GGCGTAATAGCGAAGAGGCCCGCACCGATCGCCCTTCCCAACAGTTGCGCAGCCTG
AATGGCGAATGGACGCGCCCTGTAGCGGCGCATTAAGCGCGGCGGGTGTGGTGGTT
ACGCGCAGCGTGACCGCTACACTTGCCAGCGCCCTAGCGCCCGCTCCTTTCGCTTTC
TTCCCTTCCTTTCTCGCCACGTTCGCCGGCTTTCCCCGTCAAGCTCTAAATCGGGGGC
TCCCTTTAGGGTTCCGATTTAGTGCTTTACGGCACCTCGACCCCAAAAAACTTGATTA
GGGTGATGGTTCACGTAGTGGGCCATCGCCCTGATAGACGGTTTTTCGCCCTTTGAC
GTTGGAGTCCACGTTCTTTAATAGTGGACTCTTGTTCCAAACTGGAACAACACTCAA
CCCTATCTCGGTCTATTCTTTTGATTTATAAGGGATTTTGCCGATTTCGGCCTATTGG
TTAAAAAATGAGCTGATTTAACAAAAATTTAACGCGAATTTTAACAAAATATTAACG
CTTACAATTTCCTGATGCGGTATTTTCTCCTTACGCATCTGTGCGGTATTTCACACCG
CATATGGTGCACTCTCAGTACAATCTGCTCTGATGCCGCATAGTTAAGCCAGCCCCG
ACACCCGCCAACACCCGCTGACGCGCCCTGACGGGCTTGTCTGCTCCCGGCATCCGC
TTACAGACAAGCTGTGACCGTCTCCGGGAGCTGCATGTGTCAGAGGTTTTCACCGTC
ATCACCGAAACGCGCGAGACGAAAGGGCCTCGTGATACGCCTATTTTTATAGGTTAA
TGTCATGATAATAATGGTTTCTTAGACGTCAGGTGGCACTTTTCGGGGAAATGTGCG
CGGAACCCCTATTTGTTTATTTTTCTAAATACATTCAAATATGTATCCGCTCATGAGA
CAATAACCCTGATAAATGCTTCAATAATATTGAAAAAGGAAGAGTAATTGAAACAA
GATGGATTGCACGCAGGTTCTCCGGCCGCTTGGGTGGAGAGGCTATTCGGCTATGAC
TGGGCACAACAGACAATCGGCTGCTCTGATGCCGCCGTGTTCCGGCTGTCAGCGCAG
GGGCGCCCGGTTCTTTTTGTCAAGACCGACCTGTCCGGTGCCCTGAATGAACTGCAG
GACGAGGCAGCGCGGCTATCGTGGCTGGCCACGACGGGCGTTCCTTGCGCAGCTGT
GCTCGACGTTGTCACTGAAGCGGGAAGGGACTGGCTGCTATTGGGCGAAGTGCCGG
GGCAGGATCTCCTGTCATCTCACCTTGCTCCTGCCGAGAAAGTATCCATCATGGCTG
ATGCAATGCGGCGGCTGCATACGCTTGATCCGGCTACCTGCCCATTCGACCACCAAG
CGAAACATCGCATCGAGCGAGCACGTACTCGGATGGAAGCCGGTCTTGTCGATCAG
GATGATCTGGACGAAGAGCATCAGGGGCTCGCGCCAGCCGAACTGTTCGCCAGGCT
CAAGGCGCGCATGCCCGACGGCGAGGATCTCGTCGTGACCCATGGCGATGCCTGCT
TGCCGAATATCATGGTGGAAAATGGCCGCTTTTCTGGATTCATCGACTGTGGCCGGC
TGGGTGTGGCGGACCGCTATCAGGACATAGCGTTGGCTACCCGTGATATTGCTGAAG
AGCTTGGCGGCGAATGGGCTGACCGCTTCCTCGTGCTTTACGGTATCGCCGCTCCCG
ATTCGCAGCGCATCGCCTTCTATCGCCTTCTTGACGAGTTCTTCTGAGTAACCGTCAG
ACCAAGTTTACTCATATATACTTTAGATTGATTTAAAACTTCATTTTTAATTTAAAAG
GATCTAGGTGAAGATCCTTTTTGATAATCTCATGACCAAAATCCCTTAACGTGAGTT
TTCGTTCCACTGAGCGTCAGACCCCGTAGAAAAGATCAAAGGATCTTCTTGAGATCC
TTTTTTTCTGCGCGTAATCTGCTGCTTGCAAACAAAAAAACCACCGCTACCAGCGGT
GGTTTGTTTGCCGGATCAAGAGCTACCAACTCTTTTTCCGAAGGTAACTGGCTTCAG
CAGAGCGCAGATACCAAATACTGTTCTTCTAGTGTAGCCGTAGTTAGGCCACCACTT
CAAGAACTCTGTAGCACCGCCTACATACCTCGCTCTGCTAATCCTGTTACCAGTGGC
TGCTGCCAGTGGCGATAAGTCGTGTCTTACCGGGTTGGACTCAAGACGATAGTTACC
GGATAAGGCGCAGCGGTCGGGCTGAACGGGGGGTTCGTGCACACAGCCCAGCTTGG
AGCGAACGACCTACACCGAACTGAGATACCTACAGCGTGAGCTATGAGAAAGCGCC
ACGCTTCCCGAAGGGAGAAAGGCGGACAGGTATCCGGTAAGCGGCAGGGTCGGAA
CAGGAGAGCGCACGAGGGAGCTTCCAGGGGGAAACGCCTGGTATCTTTATAGTCCT
GTCGGGTTTCGCCACCTCTGACTTGAGCGTCGATTTTTGTGATGCTCGTCAGGGGGG
CGGAGCCTATGGAAAAACGCCAGCAACGCGGCCTTTTTACGGTTCCTGGCCTTTTGC
TGGCCTTTTGCTCACATGTTCTTTCCTGCGTTATCCCCTGATTCTGTGGATAACCGTA
TTACCGCCTTTGAGTGAGCTGATACCGCTCGCCGCAGCCGAACGACCGAGCGCAGC
GAGTCAGTGAGCGAGGAAGCGGAAGAGCGCCCAATACGCAAACCGCCTCTCCCCGC
GCGTTGGCCGATTCATTAATGCAGGGCTGCAGCATGCCTGCAGGCAGCTGCGCGCTC
GCTCGCTCACTGAGGCCGCCCGGGCGTCGGGCGACCTTTGGTCGCCCGGCCTCAGTG
AGCGAGCGAGCGCGCAGAGAGGGAGTGGCCAACTCCATCACTAGGGGTTCCTGCGG
CCGCACGCGTGCTCCGGTGCCCGTCAGTGGGCAGAGCGCACATCGCCCACAGTCCC
CGAGAAGTTGGGGGGAGGGGTCGGCAATTGAACCGGTGCCTAGAGAAGGTGGCGC
GGGGTAAACTGGGAAAGTGATGTCGTGTACTGGCTCCGCCTTTTTCCCGAGGGTGGG
GGAGAACCGTATATAAGTGCAGTAGTCGCCGTGAACGTTCTTTTTCGCAACGGGTTT
GCCGCCAGAACACAGCTGGTGGGGTAGGGATGAGGGAGGGAGGGGCATTGTGATGT
ACAGGGCTGCTCTGTGAGATCAAGGGTCTCTTAAGGGTGGGAGCTGGGGCAGGGAC
TACGAGAGCAGCCAGATGGGCTGAAAGTGGAACTCAAGGGGTTTCTGGCACCTACC
TACCTGCTTCCCGCTGGGGGGTGGGGAGTTGGCCCAGAGTCTTAAGATTGGGGCAG
GGTGGAGAGGTGGGCTCTTCCTGCTTCCCACTCATCTTATAGCTTTCTTTCCCCAGAT
CCGAATTCGAGATCCAAACCAAGGAGGAAAGGATATCACAGAGGAGACCTCGAGC
CCATCAACAAGTTTGTACAAAAAAGCAGGCACCATGCTGCCGTGCCTCGTAGTGCTG
CTGGCGGCGCTCCTCAGCCTCCGTCTTGGCTCAGACGCTCATGGGACAGAGCTGCCC
AGCCCTCCGTCTGTGTGGTTTGAAGCAGAATTTTTCCACCACATCCTCCACTGGACA
CCCATCCCAAATCAGTCTGAAAGTACCTGCTATGAAGTGGCGCTCCTGAGGTATGGA
ATAGAGTCCTGGAACTCCATCTCCAACTGTAGCCAGACCCTGTCCTATGACCTTACC
GCAGTGACCTTGGACCTGTACCACAGCAATGGCTACCGGGCCAGAGTGCGGGCTGT
GGACGGCAGCCGGCACTCCAACTGGACCGTCACCAACACCCGCTTCTCTGTGGATG
AAGTGACTCTGACAGTTGGCAGTGTGAACCTAGAGATCCACAATGGCTTCATCCTCG
GGAAGATTCAGCTACCCAGGCCCAAGATGGCCCCCGCAAATGACACATATGAAAGC
ATCTTCAGTCACTTCCGAGAGTATGAGATTGCCATTCGCAAGGTGCCGGGAAACTTC
ACGTTCACACACAAGAAAGTAAAACATGAAAACTTCAGCCTCCTAACCTCTGGAGA
AGTGGGAGAGTTCTGTGTCCAGGTGAAACCATCTGTCGCTTCCCGAAGTAACAAGG
GGATGTGGTCTAAAGAGGAGTGCATCTCCCTCACCAGGCAGTATTTCACCGTGACCA
ACGTCATCATCTTCTTTGCCTTTGTCCTGCTGCTCTCCGGAGCCCTCGCCTACTGCCT
GGCCCTCCAGCTGTATGTGCGGCGCCGAAAGAAGCTACCCAGTGTCCTGCTCTTCAA
GAAGCCCAGCCCCTTCATCTTCATCAGCCAGCGTCCCTCCCCAGAGACCCAAGACAC
CATCCACCCGCTTGATGAGGAGGCCTTTTTGAAGGTGTCCCCAGAGCTGAAGAACTT
GGACCTGCACGGCAGCACAGACAGTGGCTTTGGCAGCACCAAGCCATCCCTGCAGA
CTGAAGAGCCCCAGTTCCTCCTCCCTGACCCTCACCCCCAGGCTGACAGAACGCTGG
GAAACGGGGAGCCCCCTGTGCTGGGGGACAGCTGCAGTAGTGGCAGCAGCAATAGC
ACAGACAGCGGGATCTGCCTGCAGGAGCCCAGCCTGAGCCCCAGCACAGGGCCCAC
CTGGGAGCAACAGGTGGGGAGCAACAGCAGGGGCCAGGATGACAGTGGCATTGAC
TTAGTTCAAAACTCTGAGGGCCGGGCTGGGGACACACAGGGTGGCTCGGCCTTGGG
CCACCACAGTCCCCCGGAGCCTGAGGTGCCTGGGGAAGAAGACCCAGCTGCTGTGG
CATTCCAGGGTTACCTGAGGCAGACCAGATGTGCTGAAGAGAAGGCAACCAAGACA
GGCTGCCTGGAGGAAGAATCGCCCTTGACAGATGGCCTTGGCCCCAAATTCGGGAG
ATGCCTGGTTGATGAGGCAGGCTTGCATCCACCAGCCCTGGCCAAGGGCTATTTGAA
ACAGGATCCTCTAGAAATGACTCTGGCTTCCTCAGGGGCCCCAACGGGACAGTGGA
ACCAGCCCACTGAGGAATGGTCACTCCTGGCCTTGAGCAGCTGCAGTGACCTGGGA
ATATCTGACTGGAGCTTTGCCCATGACCTTGCCCCTCTAGGCTGTGTGGCAGCCCCA
GGTGGTCTCCTGGGCAGCTTTAACTCAGACCTGGTCACCCTGCCCCTCATCTCTAGC
CTGCAGTCAAGTGAGCGGGCCAAGCGGCAGCTGCTGAACTTCGACCTGCTGAAGCT
GGCCGGCGACGTGGAGAGCAACCCCGGGCCCATGCACAGCTCAGCACTGCTCTGTT
GCCTGGTCCTCCTGACTGGGGTGAGGGCCAGCCCAGGCCAGGGCACCCAGTCTGAG
AACAGCTGCACCCACTTCCCAGGCAACCTGCCTAACATGCTTCGAGATCTCCGAGAT
GCCTTCAGCAGAGTGAAGACTTTCTTTCAAATGAAGGATCAGCTGGACAACTTGTTG
TTAAAGGAGTCCTTGCTGGAGGACTTTAAGGGTTACCTGGGTTGCCAAGCCTTGTCT
GAGATGATCCAGTTTTACCTGGAGGAGGTGATGCCCCAAGCTGAGAACCAAGACCC
AGACATCAAGGCGCATGTGAACTCCCTGGGGGAGAACCTGAAGACCCTCAGGCTGA
GGCTACGGCGCTGTCATCGATCTCTTCCCTGTGAAAACAAGAGCAAGGCCGTGGAG
CAGGTGAAGAATGCCTTTAATAAGCTCCAAGAGAAAGGCATCTACAAAGCCATGAG
TGAGTTTGACATCTTCATCAACTACATAGAAGCCTACATGACAATGAAGATACGAAA
CTGAACCCAGCTTTCTTGTACAAAGTGGTGCTAGCACTCTCAGTACAATCTGCTCTG
ATGCCGCATAGTTAAGCCAGTATCTGCTCCCTGCTTGTGTGTTGGAGGTCGCTGAGT
AGTGCGCGAGCAAAATTTAAGCTACAACAAGGCAAGGCTTGACCGACAATTGCATG
AAGAATCTGCTTAGGGTTAGGCGTTTTGCGCTGCTTCGCGATGTACGGGCCAGATAT
ACGCGTACCGGTTAGTAATGATCGACAATCAACCTCTGGATTACAAAATTTGTGAAA
GATTGACTGGTATTCTTAACTATGTTGCTCCTTTTACGCTATGTGGATACGCTGCTTT
AATGCCTTTGTATCATGCTATTGCTTCCCGTATGGCTTTCATTTTCTCCTCCTTGTATA
AATCCTGGTTGCTGTCTCTTTATGAGGAGTTGTGGCCCGTTGTCAGGCAACGTGGCG
TGGTGTGCACTGTGTTTGCTGACGCAACCCCCACTGGTTGGGGCATTGCCACCACCT
GTCAGCTCCTTTCCGGGACTTTCGCTTTCCCCCTCCCTATTGCCACGGCGGAACTCAT
CGCCGCCTGCCTTGCCCGCTGCTGGACAGGGGCTCGGCTGTTGGGCACTGACAATTC
CGTGGTGTTGTCGGGGAAATCATCGTCCTTTCCTTGGCTGCTCGCCTATGTTGCCACC
TGGATTCTGCGCGGGACGTCCTTCTGCTACGTCCCTTCGGCCCTCAATCCAGCGGAC
CTTCCTTCCCGCGGCCTGCTGCCGGCTCTGCGGCCTCTTCCGCGTCTTCGCCTTCGCC
CTCAGACGAGTCGGATCTCCCTTTGGGCCGCCTCCCCGCCTGGCGATGGTACCCAAT
TCGCCCTATAGTGAGTCGTATTACGCGCGCAGCGGCCGACCATGGCCCAACTTGTTT
ATTGCAGCTTATAATGGTTACAAATAAAGCAATAGCATCACAAATTTCACAAATAAA
GCATTTTTTTCACTGCATTCTAGTTGTGGTTTGTCCAAACTCATCAATGTATCTTATC
ATGTCTGGATCTCCGGACACGTGCGGACCGAGCGGCCGCAGGAACCCCTAGTGATG
GAGTTGGCCACTCCCTCTCTGCGCGCTCGCTCGCTCACTGAGGCCGGGCGACCAAAG
GTCGCCCGACGCCCGGGCTTTGCCCGGGCGGCCTCAGTGAGCGAGCGAGCGCGCAG
CTGCCTGCAGGTCTGAGACAATAACCCTGATAAATGCTTCAATAATGTA.

In some embodiments, the vector comprises the nucleotide sequence of SEQ ID NO: 2. In certain embodiments, the vector consists of the nucleotide sequence of SEQ ID NO: 2. In certain embodiments, the vector comprises one mutation relative to the nucleotide sequence of SEQ ID NO: 2. In certain embodiments, the mutation comprises one or more deletions. In some embodiments, the mutation comprises one or more insertions. In certain embodiments, the mutation comprises insertions and deletions. In some embodiments, the mutation comprises one or more insertions and deletions. In some embodiments, the mutation comprises a point mutation with a single amino acid has been replaced (i.e., deleted then inserted). In certain embodiments, the vector comprises one or more, two or more, three or more, four or more, five or more, six or more, seven or more, eight or more, nine or more, or ten or more mutations relative to the nucleotide sequence of SEQ ID NO: 2.

In some embodiments, the plasmid used herein encodes a IL-10 polypeptide. In some embodiments, the IL-10 polypeptide is wild type IL-10. In some embodiments, the IL-10 polypeptide comprises a mutation. In some embodiments, the mutation is within the portion of the IL-10 polypeptide comprising: LRRCHRFLPCENKSK (SEQ ID NO: 26). In some embodiments, the mutation is within the portion of the IL-10 polypeptide: LRRCHRFLPCENKSK (SEQ ID NO: 26) of any one of SEQ ID: 3 through SEQ ID: 22, or any other IL-10 peptide. In certain embodiments, the mutation is within the portion of the IL-10 polypeptide: LRRCHRFLPCENKSK (SEQ ID NO: 26) of any one of SEQ ID:3 through SEQ ID: 22. In some embodiments, the mutation is within the portion of the IL-10 polypeptide: LRRCHRFLPCENKSK (SEQ ID NO: 26) of SEQ ID: 3, SEQ ID: 7, SEQ ID: 8, or SEQ ID: 21. In certain embodiments, the mutation is within the portion of the IL-10 polypeptide: LRRCHRFLPCENKSK (SEQ ID NO: 26) of SEQ ID: 4 through SEQ ID: 6, SEQ ID: 9, SEQ ID: 11-19, SEQ ID: 22-25. In some embodiments, the mutation is a substitution of serine for phenylalanine at: (i) position 129 of any one of SEQ ID: 3 through SEQ ID: 18 or SEQ ID: 23-25; (ii) position 130 of SEQ ID: 19 or SEQ ID: 20; (iii) position 132 of SEQ ID: 21; or (iv) position 171 of SEQ ID: 22. In some embodiments, the mutation is a substitution of serine for phenylalanine at position 129 of any one of SEQ ID: 3 through SEQ ID: 18 or SEQ ID: 23-25. In some embodiments, the mutation is a substitution of serine for phenylalanine at position 130 of SEQ ID: 19 or SEQ ID: 20. In some embodiments, the mutation is a substitution of serine for phenylalanine at position 132 of SEQ ID: 21. In some embodiments, the mutation is a substitution of serine for phenylalanine at position 171 of SEQ ID: 22. In some embodiments, the plasmid encodes a polypeptide at least 99%, 98%, 95%, 90%, 85%, and 80% identical to any one of SEQ ID NO: 3 through SEQ ID: 25.

In some embodiments, the plasmid encodes a protein with an amino acid sequence at least 100%, 99%, 98%, 95%, 90%, 85%, or 80% identical to an amino acid sequence native to a mammal. In certain embodiments, the plasmid encodes an amino sequence at least 100%, 99%, 98%, 95%, 90%, 85%, or 80% identical to an amino acid sequence native to a human or non-human animal. In certain embodiments, the plasmid encodes an amino sequence at least 100%, 99%, 98%, 95%, 90%, 85%, or 80% identical to an amino acid sequence native to a human. In certain embodiments, the plasmid encodes an amino sequence at least 100%, 99%, 98%, 95%, 90%, 85%, or 80% identical to an amino acid sequence native to a pet. In certain embodiments, the plasmid encodes an amino sequence at least 100%, 99%, 98%, 95%, 90%, 85%, or 80% identical to an amino acid sequence native to a farm animal. In certain embodiments, the plasmid encodes an amino sequence at least 100%, 99%, 98%, 95%, 90%, 85%, or 80% identical to an amino acid sequence native to a research animal. In certain embodiments, the plasmid encodes an amino sequence at least 100%, 99%, 98%, 95%, 90%, 85%, or 80% identical to an amino acid sequence native to a human, dog, horse, rats, mouse, cat, guinea pig, llama, gerbil, goat, sheep, ferret, rabbit, hamster, or monkey (e.g., chimp, macaques, marmoset, cynomolgus monkey, squirrel monkey). In certain embodiments, the plasmid encodes an amino sequence at least 100%, 99%, 98%, 95%, 90%, 85%, or 80% identical to an amino acid sequence native to a human, dog, horse, rats, mouse, cat, or dog. In certain embodiments, the plasmid encodes an amino sequence at least 100%, 99%, 98%, 95%, 90%, 85%, or 80% identical to an amino acid sequence native to a human, dog, horse, cat, or dog.

In some embodiments, the plasmid encodes a protein with an amino acid sequence at least 100%, 99%, 98%, 95%, 90%, 85%, or 80% identical to an amino acid sequence native to a mammal. In certain embodiments, the plasmid encodes an amino sequence at least 100%, 99%, 98%, 95%, 90%, 85%, or 80% identical to an amino acid sequence native to a human or non-human animal. In certain embodiments, the plasmid encodes an amino sequence at least 100%, 99%, 98%, 95%, 90%, 85%, or 80% identical to an amino acid sequence native to a human. In certain embodiments, the plasmid encodes an amino sequence at least 100%, 99%, 98%, 95%, 90%, 85%, or 80% identical to an amino acid sequence native to a pet. In certain embodiments, the plasmid encodes an amino sequence at least 100%, 99%, 98%, 95%, 90%, 85%, or 80% identical to an amino acid sequence native to a farm animal. In certain embodiments, the plasmid encodes an amino sequence at least 100%, 99%, 98%, 95%, 90%, 85%, or 80% identical to an amino acid sequence native to a research animal. In certain embodiments, the plasmid encodes an amino sequence at least 100%, 99%, 98%, 95%, 90%, 85%, or 80% identical to an amino acid sequence native to a human, dog, horse, rats, mouse, cat, guinea pig, llama, gerbil, goat, sheep, ferret, rabbit, hamster, or monkey (e.g., chimp, macaques, marmoset, cynomolgus monkey, squirrel monkey). In certain embodiments, the plasmid encodes an amino sequence at least 100%, 99%, 98%, 95%, 90%, 85%, or 80% identical to an amino acid sequence native to a human, dog, horse, rats, mouse, cat, or dog. In certain embodiments, the plasmid encodes an amino sequence at least 100%, 99%, 98%, 95%, 90%, 85%, or 80% identical to an amino acid sequence native to a human, dog, horse, cat, or dog.

Human IL-10:
(SEQ ID NO: 3)
MHSSALLCCLVLLTGVRASPGQGTQSENSCTHFPGNLPNMLRDLR
DAFSRVKTFFQMKDQLDNLLLKESLLEDFKGYLGCQALSEMIQFYLEEVMPQAENQDP
DIKAHVNSLGENLKTLRLRLRRCHRFLPCENKSKAVEQVKNAFNKLQEKGIYKAMSEF
DIFINYIEAYMTMKIRN
Rat IL-10:
(SEQ ID NO: 4)
MPGSALLCCLLLLAGVKTSKGHSIRGDNNCTHFPVSQTHMLRELRAA
FSQVKTFFQKKDQLDNILLTDSLLQDFKGYLGCQALSEMIKFYLVEVMPQAENHGPEIK
EHLNSLGEKLKTLWIQLRRCHRFLPCENKSKAVEQVKNDFNKLQDKGVYKAMNEFDIFI
NCIEAYVTLKMKN
Rat IL-10:
(SEQ ID NO: 5)
MLGSALLCCLLLLAGVKTSKGHSIRGDNNCTHFPVSQTHMLRELRAA
FSQVKTFFQKKDQLDNIVLTDSLLQDFKGYLGCQALSEMIKFYLVEVMPQAENHGPEIK
EHLNSLGEKLKTLWIQLRRCHRFLPCENKSKAVEQVKNDFNKLQDKGVYKAMNEFDIFI
NCIEAYVTLKMKN
Mouse IL-10:
(SEQ ID NO: 6)
MPGSALLCCLLLLTGMRISRGQYSREDNNCTHFPVGQSHMLLELR
TAFSQVKTFFQTKDQLDNILLTDSLMQDFKGYLGCQALSEMIQFYLVEVMPQAEKHGPE
IKEHLNSLGEKLKTLRMRLRRCHRFLPCENKSKAVEQVKSDFNKLQDQGVYKAMNEFD
IFINCIEAYMMIKMKS
Horse IL-10:
(SEQ ID NO: 7)
MHSSALLCYLVFLAGVGASRDRGTQSENSCTHFPTSLPHMLHELRA
AFSRVKTFFQMKDQLDNMLLNGSLLEDFKGYLGCQALSEMIQFYLEEVMPQAENHGPD
IKEHVNSLGEKLKTLRVRLRRCHRFLPCENKSKAVEQVKSAFSKLQEKGVYKAMSEFDI
FINYIEAYMTTKMKN
Cat IL-10:
(SEQ ID NO: 8)
MHSSALLCFLVFLAGVGASRHQSTLSEDNCTHESVSLPHMLRELRAAF
GKVKTFFQTKDELHSILLTRSLLEDFKGYLGCQALSEMIQFYLEEVMPQAENEDPDIKQH
VNSLGEKLKTLRLRLRRCHRFLPCENKSKVVEQVKSTFSKLQEKGVYKAMGEFDIFINYI
EAYMTMKMKI
Guinea pig IL-10:
(SEQ ID NO: 9)
MPGSALLCCLALLAGVKASQGTNTQSEDSCAHFPAGLPHM
LRELRAAFGRVKTFFQTQDQLDNVLLNKSLLEDFKGYLGCQALSEMIQFYLVEVMPQA
EKHGPEIKEHLNSLGEKLKTLRMRLRRCHRFLPCENKSKAVEQVKSDFNKLQDQGVYK
AMNEFDIFINCIEAYMMIKMKS
Llama IL-10:
(SEQ ID NO: 10)
MPRSALLCCLILLAGVAASRDQGTQSENSCAHFPASLPHMLRELRA
AFGRVKTFFQMKDQLDNMLLTRSLLEDFKGYLGCQALSEMIQFYLEEVMPQAENHGPD
IKEHVNSLGEKLKTLRLRLRRCHRFLPCENKSKAVEQVRGVFSKLQEKGVYKAMSEFDI
FINYIEAYMTMKMKN
Gerbil IL-10:
(SEQ ID NO: 11)
MPSSALLYCLILLAGVRPSRGEYPRNESNCTHFPVSQTHMLRELRA
AFSQVKTFFQKKDQLDNILLTDSLQQDFKGYLGCQALSEMIQFYLVEVMPQAENHGPEI
KENLNSLGEKLKTLRMQLRRCHRFLPCENKSKAVEQVKNDFNKLQEKGVYKAMNEFDI
FINCIEAYMTIKMKS
Chimp IL-10:
(SEQ ID NO: 12)
MHSSALLCCLVLLTGVRASPGQGTQSENSCTHFPGNLPNMLRDLR
DAFSRVKTFFQMKDQLDNLLLKESLLEDFKGYLGCQALXEMIQFYLEEVMPQAENQDP
DIKVHVNSLGENLKTLRLRLRRCHRFLPCENKSKAVEQVKNAFNKLQEKGIYKAMSEF
DIFINYIEAYMTMKIRN
Rhesus macaques IL-10:
(SEQ ID NO: 13)
MHSSALLCCLVLLTGVRASPGQGTQSENSCTRFPGNLPH
MLRDLRDAFSRVKTFFQMKDQLDNILLKESLLEDFKGYLGCQALSEMIQFYLEEVMPQ
AENHDPDIKEHVNSLGENLKTLRLRLRRCHRFLPCENKSKAVEQVKNAFSKLQEKGVY
KAMSEFDIFINYIEAYMTMKIQN
Marmoset IL-10:
(SEQ ID NO: 14)
MHSSALLCCLVFLTGVRASPGQGTQSENSCTHFPGSLPHMLREL
RVAFSRVKTFFQKKDQLDSMLLKESLLEDFKGYLGCQALSEMIQFYLEEVMPQAENHD
PDIKEHVNSLGEKLKTFRLRLRRCHRFLPCENKSKAVVQVKNAVSKLQEKGIYKAMSEF
DIFIDYIEAYMTMKAQN
Pig-tailed macaque IL-10:
(SEQ ID NO: 15)
MHSSALLCCLVLLTGVRASPGQGTQSENSCTRFPGNLP
HMLRDLRDAFSRVKTFFQMKDQLDNILLKESLLEDFKGYLGCQALSEMIQFYLEEVMP
QAENHDPDIKEHVNSLGENLKTLRLRLRRCHRFLPCENKSKAVEQVMNAFSKLQEKGV
YKAMSEFDIFINYIEAYMTMKIQN
Cynomolgus monkey IL-10:
(SEQ ID NO: 16)
MHSSALLCCLVLLTGVRASPGQGTQSENSCTRFPG
NLPHMLRDLRDAFSRVKTFFQMKDQLDNILLKESLLEDFKGYLGCQALSEMIQFYLEEV
MPQAENHDPDIKEHVNSLGENLKTLRLRLRRCHRFLPCENKSKAVEQVKNAFSKLQEK
GVYKAMSEFDIFINYIEAYMTMKIRN
Sooty mangabey IL-10:
(SEQ ID NO: 17)
MHSSALLCCLVLLTGVRASPGQGTQSENSCTRFPGNLPH
MLRDLRDVFSRVKTFFQMKDQLDNILLKESLLEDFKGYLGCQALSEMIQFYLEEVMPQ
AENHDPDIKEHVNSLGENLKTLRLRLRRCHRFLPCENKSKAVEQVKNAFSKLQEKGVY
KAMSEFDIFINYIEAYMTMKIQN
Squirrel monkey IL-10:
(SEQ ID NO: 18)
MHSSALLCCLVFLTGVRASPGQGTQSENSCTHFPGSLPH
MLRELRVAFGRVKTFFQKKDQLDSMLLKESLLEDFKGYLGCQALSEMIQFYLEEVMPQ
AENHDPDIKEHVNSLGEKLKTFRLRLRRCHRFLPCENKSKAVAQVKNAVSKLQEKGVY
KAMSEFDIFIDYIEAYMTMKTQN
Goat IL-10:
(SEQ ID NO: 19)
MPSSSALLCCLVFLAGVAASRDASTLSDSSCTHFPASLPHMLRELRA
AFGKVKTFFQMKDQLNSMLLTQSLLDDFKGYLGCQALSEMIQFYLEEVMPQAENHGPD
IKEHVNSLGEKLKTLRLRLRRCHRFLPCENKSKAVEQVKRVFNMLQERGVYKAMSEFDI
FINYIESYMTTKM
Sheep IL-10:
(SEQ ID NO: 20)
MPSSSAVLCCLVFLAGVAASRDASTLSDSSCTHFPASLPHMLRDVR
AAFGKVKTFFQMKDQLNSMLLTQSLLDDFKGYLGCQALSEMIQFYLEEVMPQAENHGP
DIKEHVNSLGEKLKTLRLRLRRCHRFLPCENKSKAVEQVKRVFNMLQERGVYKAMSEF
DIFINYIESYMTTKM
Dog IL-10:
(SEQ ID NO: 21)
MHGSALLCCCLVLLAGVGASRHQSTLLEDDCTHFPASLPHMLRELR
AAFGRVKIFFQMKDKLDNILLTGSLLEDFKSYLGCQALSEMIQFYLEEVMPRAENHDPDI
KNHVNSLGEKLKTLRLRLRLRRCHRFLPCENKSKAVEQVKSAFSKLQEKGVYKAMSEF
DIFINYIETYMTMRMKI
Ferret IL-10:
(SEQ ID NO: 22)
MNGEASPHPHIKGGPVGEGLHTRGLLLQHQTTRPALGRQSFTMPSP
ALLCCLVLLAGVGASRHQSALSEDNCTHFPASLPHMLRELRAAFGRVKTFFQMKDKLG
SILLTGSLLEDFKGYLGCQALSEMIQFYLEEVMPQAENHDPEVKEHVNSLGEKLKTLRL
RLRRCHRFLPCENKSKAVEQVKSAFSKLQERGVYKAMSEFDIFINYIETYMTMRMKI
Rabbit IL-10:
(SEQ ID NO: 23)
MLSSALLCCLVFLGGTGASRGQDTPAENSCIHFPGGLPHMLRELRA
AFGRVKTFFQSKDQLNSMLLTESLLEDLKGYLGCQALSEMIQFYLKDVMPQAENHSPAI
REHVNSLGENLKTLRLRLRQCHRFLPCENKSKAVEQVKSAFSKLQEEGVYKAMSEFDIF
INYIETYMTMKIKS
Golden hamster IL-10:
(SEQ ID NO: 24)
MLGSALLCCLLLLAGVGPSRGQYTQHESNCTHFPVSQTH
MLRELRTAFSQVKTFFQKKDQLDNILLTDSLLQDFKGYLGCQTLSEMIQFYLVEVMPQA
ENHGPEIKEHLNSLGEKLKTLRRQLQRCHRFLPCENKSKAVEQVKDNFNKLQEKGVFK
AMNEFDIFINCIEVYMTIKMKS
Chinese Hamster IL-10:
(SEQ ID NO: 25)
MPVSALLCCLLLLAGVGPSRGQYTQQENNCTHFPVSQTH
MLRELRTAFSQVKTFFQKKDQLDNILLTDSLVQDFEGYLGCQTLSEMIQFYLVEVMPQA
ENHGPEIKEHLNSLGEKLKTLRRQLQRCHRFLPCENKSKAVEKVKSDFNKLQEKGVYK
AMNEFDIFINCIETYMTIKMKS
Human IL-10^{F129S}:
(SEQ ID NO: 27)
ATGCACAGCTCAGCACTGCTCTGTTGCCTGGTCCTCCTGAC
TGGGGTGAGGGCCAGCCCAGGCCAGGGCACCCAGTCTGAGAACAGCTGCACCCACT
TCCCAGGCAACCTGCCTAACATGCTTCGAGATCTCCGAGATGCCTTCAGCAGAGTGA
AGACTTTCTTTCAAATGAAGGATCAGCTGGACAACTTGTTGTTAAAGGAGTCCTTGC
TGGAGGACTTTAAGGGTTACCTGGGTTGCCAAGCCTTGTCTGAGATGATCCAGTTTT
ACCTGGAGGAGGTGATGCCCCAAGCTGAGAACCAAGACCCAGACATCAAGGCGCAT
GTGAACTCCCTGGGGGAGAACCTGAAGACCCTCAGGCTGAGGCTACGGCGCTGTCA
TCGATCTCTTCCCTGTGAAAACAAGAGCAAGGCCGTGGAGCAGGTGAAGAATGCCT
TTAATAAGCTCCAAGAGAAAGGCATCTACAAAGCCATGAGTGAGTTTGACATCTTCA
TCAACTACATAGAAGCCTACATGACAATGAAGATACGAAACTGAG
Human IL-10:
(SEQ ID NO: 28)
ATGCACAGCTCAGCACTGCTCTGTTGCCTGGTCCTCCTGACTGG
GGTGAGGGCCAGCCCAGGCCAGGGCACCCAGTCTGAGAACAGCTGCACCCACTTCC
CAGGCAACCTGCCTAACATGCTTCGAGATCTCCGAGATGCCTTCAGCAGAGTGAAG
ACTTTCTTTCAAATGAAGGATCAGCTGGACAACTTGTTGTTAAAGGAGTCCTTGCTG
GAGGACTTTAAGGGTTACCTGGGTTGCCAAGCCTTGTCTGAGATGATCCAGTTTTAC
CTGGAGGAGGTGATGCCCCAAGCTGAGAACCAAGACCCAGACATCAAGGCGCATGT
GAACTCCCTGGGGGAGAACCTGAAGACCCTCAGGCTGAGGCTACGGCGCTGTCATC
GATTTCTTCCCTGTGAAAACAAGAGCAAGGCCGTGGAGCAGGTGAAGAATGCCTTT
AATAAGCTCCAAGAGAAAGGCATCTACAAAGCCATGAGTGAGTTTGACATCTTCAT
CAACTACATAGAAGCCTACATGACAATGAAGATACGAAACTGAG

In some embodiments, first dose of the composition and/or the second dose of the composition further comprises one or more excipients. The first dose of the composition and/or the second dose of the composition may be administered to the subject with an excipient for increasing the uptake of the vector. Classes of such excipients that may be used in the methods described herein include, but are not limited to, a sugar, calcium phosphate, a dendrimer, an oligonucleotide, a high molecular weight hyaluronic acid, and a lipid. In some embodiments, the sugar is mannose, sucrose, or glucose. In some embodiments, the sugar is D-mannose, sucrose, or glucose. In certain embodiments, the excipient is D-mannose. In certain embodiments, the excipient is sucrose. In certain embodiments, the excipient is a combination of D-mannose and sucrose. In some embodiments, the composition is formulated to include such an excipient (i.e., such that the excipient is administered concurrently with the composition).

The composition may also further comprise various other excipients or diluents. In some embodiments, the diluent is or comprises phosphate-buffered saline. Additional exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof. In some embodiments, the composition further comprises a diluent. In some embodiments, the diluent is phosphate-buffered saline (pH 7.4).

The volume of the composition administered to a joint depends on the joint. In some embodiments, the first and/or second dose of the administered composition is formulated in a solution, wherein the volume of administration is approximately 1 mL. In some embodiments, the volume of composition administered to a subject is approximately 1 mL+/−5%. In some embodiments, the volume of composition administered to a subject is approximately 0.01 mL+/−5%. In some embodiments, the volume of composition administered to a subject is approximately 0.05 mL+/−5%. In some embodiments, the volume of composition administered to a subject is approximately 0.1 mL+/−5%. In some embodiments, the volume of composition administered to a subject is approximately 0.5 mL+/−5%. In some embodiments, the volume of composition administered to a subject is approximately 1.5 mL+/−5%.

In some embodiments, the first and/or the second dose of the composition further comprises phosphate-buffered saline, sucrose. and D-mannose. In some embodiments, the first and/or the second dose of the composition is formulated in a solution of phosphate-buffered saline with sucrose and D-mannose. In some embodiments, the composition comprises about 3% w/v sucrose. In some embodiments, the composition comprises about 2.5 mg/mL D-mannose. In certain embodiments, the first and/or the second dose of the composition is formulated in a solution of phosphate-buffered saline (pH 7.4) with 3% w/v sucrose and 2.5 mg/mL D-mannose.

In some embodiments, the composition comprises about 0.05-0.6 mg/mL of IL-10^{F129S} expression construct. In some embodiments, the composition comprises about 0.1-0.5 mg/mL of IL-10^{F129S} expression construct. In some embodiments, the composition comprises about 0.15-0.45 mg/mL of IL-10^{F129S} expression construct. In some embodiments, the composition comprises about 0.15 mg/mL of IL-10^{F129S} expression construct. In some embodiments, the composition comprises about 0.45 mg/mL of IL-10^{F129S} expression construct.

In some embodiments, the first and/or the second dose of the composition comprises 100-600 μg of the IL-10^{F129S} expression construct and is formulated in a solution of phosphate-buffered saline with sucrose and D-mannose. In certain embodiments, the first and/or the second dose of the composition comprises 100-600 μg of the IL-10^{F129S} expression construct and is formulated in a solution of phosphate-buffered saline (pH 7.4) with 3% w/v sucrose and 2.5 mg/mL D-mannose.

In some embodiments, the first and/or the second dose of the composition comprises 150 or 450 μg of the IL-10^{F129S} expression construct and is formulated in a solution of phosphate-buffered saline with sucrose and D-mannose. In certain embodiments, the first and/or the second dose of the composition comprises 150 or 450 μg of the IL-10^{F129S} expression construct and is formulated in a solution of phosphate-buffered saline (pH 7.4) with 3% w/v sucrose and 2.5 mg/mL D-mannose.

In some embodiments, the first and/or the second dose of the composition comprises 0.1-0.6 mg/mL of the IL-10^{F129S} expression construct and is formulated in a solution of phosphate-buffered saline with sucrose and D-mannose. In certain embodiments, the first and/or the second dose of the composition comprises 0.1-0.6 mg/mL of the IL-10^{F129S} expression construct and is formulated in a solution of phosphate-buffered saline (pH 7.4) with 3% w/v sucrose and 2.5 mg/mL D-mannose.

In some embodiments, the first and/or the second dose of the composition comprises 150 or 450 μg of the IL-10^{F129S} expression construct and is formulated in a solution of phosphate-buffered saline with sucrose and D-mannose. In certain embodiments, the first and/or the second dose of the composition comprises 150 or 450 μg of the IL-10^{F129S} expression construct and is formulated in a solution of phosphate-buffered saline (pH 7.4) with 3% w/v sucrose and 2.5 mg/mL D-mannose.

In some embodiments, the first and/or second dose of the administered composition is formulated in a solution of phosphate-buffered saline (pH 7.4) with sucrose and D-mannose, wherein the volume of administration is approximately 1 mL. In some embodiments, the composition comprises about 2.5 mg/mL D-mannose. In certain embodiments, the first and/or second dose of the composition is formulated in a solution of phosphate-buffered saline (pH 7.4) with 3% w/v sucrose and 2.5 mg/mL D-mannose, wherein the volume of administration is approximately 1 mL.

In certain embodiments, the IL-10^{F129S} expression construct described herein is provided in the composition in an effective amount. In certain embodiments, the effective amount is a therapeutically effective amount. In certain embodiments, the effective amount is a prophylactically effective amount. In certain embodiments, the effective amount is an amount effective for treating an inflammatory joint disease (e.g., osteoarthritis) in a subject. In some embodiments, the effective amount is 100-600 μg. In some embodiments, the effective amount is 100-500 μg. In some embodiments, the effective amount is 150-450 μg. In some embodiments, the effective amount is 150 or 450 μg. In some embodiments, the effective amount is 150 μg. In some embodiments, the effective amount is 450 μg. In some embodiments, the effective amount is 0.1-0.6 mg/mL. In some embodiments, the effective amount is 0.1-0.5 mg/mL. In some embodiments, the effective amount is 0.15-0.45 mg/mL. In some embodiments, the effective amount is 0.15 or 0.45 mg/mL. In some embodiments, the effective amount is 0.15 mg/mL. In some embodiments, the effective amount is 0.45 mg/mL.

Any inflammatory joint disease, and any inflamed joint, may be treated using the methods provided herein. In some embodiments, the inflammatory joint disease is arthritis (e.g., rheumatoid arthritis or osteoarthritis), tendonitis, bursitis, inflammation of the ligament, synovitis, gout, facet syndrome, or systemic lupus erythematosus. In certain embodiments, the inflammatory joint disease is rheumatoid arthritis. In certain embodiments, the inflammatory joint disease is osteoarthritis. In some embodiments, the inflamed joint is a hand (e.g., finger), a foot (e.g., toe), a neck, a hip, a knee, an elbow, a wrist, an ankle, a hip, a shoulder, a jaw, or a spine (including, e.g., the facet joint). In some embodiments, the inflamed joint is a knee, an elbow, a wrist, an ankle, a hip, a shoulder, a jaw, or a spine (including, e.g., the facet joint). In certain embodiments, the inflamed joint is a knee. In certain embodiments, the inflammatory joint disease is osteoarthritis of the knee.

In certain embodiments, the present disclosure provides methods for treating an inflammatory joint disease in a subject comprising injecting into an inflamed joint of the subject a first dose of a composition comprising an IL-10^{F129S} expression construct, wherein the first dose of the composition comprises 450 μg of the IL-10^{F129S} expression construct; and injecting into the inflamed joint of the subject a second dose of the composition 150-360 days after injection of the first dose, wherein the second dose of the composition comprises 450 μg of the IL-10^{F129S} expression construct. In certain embodiments, the present disclosure provides a method for treating an inflammatory joint disease in a subject comprising injecting into an inflamed joint of the subject a first dose of a composition comprising an IL-10^{F129S} expression construct, wherein the first dose of the composition comprises 450 μg of the IL-10^{F129S} expression construct; and injecting into the inflamed joint of the subject a second dose of the composition about 150-180 days after injection of the first dose, wherein the second dose of the composition comprises 450 μg of the IL-10^{F129S} expression construct. In certain embodiments, the present disclosure provides a method for treating an inflammatory joint disease in a subject comprising injecting into an inflamed joint of the subject a first dose of a composition comprising an IL-10^{F129S} expression construct, wherein the first dose of the composition comprises 450 μg of the IL-10^{F129S} expression construct; and injecting into the inflamed joint of the subject a second dose of the composition 120 days after injection of the first dose, wherein the second dose of the composition comprises 450 μg of the IL-10^{F129S} expression construct. In certain embodiments, the present disclosure provides a method for treating an inflammatory joint disease in a subject comprising injecting into an inflamed joint of the subject a first dose of a composition comprising an IL-10^{F129S} expression construct, wherein the first dose of the composition comprises 450 μg of the IL-10^{F129S} expression construct; and injecting into the inflamed joint of the subject a second dose of the composition about 150 days after injection of the first dose, wherein the second dose of the composition comprises 450 μg of the IL-10^{F129S} expression construct. In certain embodiments, the present disclosure provides a method for treating an inflammatory joint disease in a subject comprising injecting into an inflamed joint of the subject a first dose of a composition comprising an IL-10^{F129S} expression construct, wherein the first dose of the composition comprises 450 μg of the IL-10^{F129S} expression construct; and injecting into the inflamed joint of the subject a second dose of the composition about 180 days after injection of the first dose, wherein the second dose of the composition comprises 450 μg of the IL-10^{F129S} expression construct. In certain embodiments, the present disclosure provides a method for treating an inflammatory joint disease in a subject comprising injecting into an inflamed joint of the subject a first dose of a composition comprising an IL-10^{F129S} expression construct, wherein the first dose of the composition comprises 450 μg of the IL-10^{F129S} expression construct; and injecting into the inflamed joint of the subject a second dose of the composition 210 days after injection of the first dose, wherein the second dose of the composition comprises 450 μg of the IL-10^{F129S} expression construct. In certain embodiments, the present disclosure provides a method for treating an inflammatory joint disease in a subject comprising injecting into an inflamed joint of the subject a first dose of a composition comprising an IL-10^{F129S} expression construct, wherein the first dose of the composition comprises 450 μg of the IL-10^{F129S} expression construct; and injecting into the inflamed joint of the subject a second dose of the composition 240 days after injection of the first dose, wherein the second dose of the composition comprises 450 μg of the IL-10^{F129S} expression construct.

In certain embodiments, the present disclosure provides methods for treating osteoarthritis in a subject comprising injecting into an inflamed joint of the subject a first dose of a composition comprising an IL-10^{F129S} expression construct, wherein the first dose of the composition comprises 450 μg of the IL-10^{F129S} expression construct; and injecting into the inflamed joint of the subject a second dose of the composition 150-360 days after injection of the first dose, wherein the second dose of the composition comprises 450 μg of the IL-10^{F129S} expression construct. In certain embodiments, the present disclosure provides a method for treating osteoarthritis in a subject comprising injecting into an inflamed joint of the subject a first dose of a composition comprising an IL-10^{F129S} expression construct, wherein the first dose of the composition comprises 450 μg of the IL-10^{F129S} expression construct; and injecting into the inflamed joint of the subject a second dose of the composition 120 days after injection of the first dose, wherein the second dose of the composition comprises 450 μg of the IL-10^{F129S} expression construct. In certain embodiments, the present disclosure provides a method for treating osteoarthritis in a subject comprising injecting into an inflamed joint of the subject a first dose of a composition comprising an IL-10^{F129S} expression construct, wherein the first dose of the composition comprises 450 μg of the IL-10^{F129S} expression construct; and injecting into the inflamed joint of the subject a second dose of the composition 150 days after injection of the first dose, wherein the second dose of the composition comprises 450 μg of the IL-10^{F129S} expression construct. In certain embodiments, the present disclosure provides a method for treating osteoarthritis in a subject comprising injecting into an inflamed joint of the subject a first dose of a composition comprising an IL-10^{F129S} expression construct, wherein the first dose of the composition comprises 450 μg of the IL-10^{F129S} expression construct; and injecting into the inflamed joint of the subject a second dose of the composition 180 days after injection of the first dose, wherein the second dose of the composition comprises 450 μg of the IL-10^{F129S} expression construct. In certain embodiments, the present disclosure provides a method for treating osteoarthritis in a subject comprising injecting into an inflamed joint of the subject a first dose of a composition comprising an IL-10^{F129S} expression construct, wherein the first dose of the composition comprises 450 μg of the IL-10^{F129S} expression construct; and injecting into the inflamed joint of the subject a second dose of the composition 210 days after injection of the first dose, wherein the second dose of the composition comprises 450 μg of the IL-10^{F129S} expression construct. In certain embodiments, the present disclosure provides a method for treating osteoarthritis in a subject comprising injecting into an inflamed joint of the subject a first dose of a composition comprising an IL-10^{F129S} expression construct, wherein the first dose of the composition comprises 450 μg of the IL-10^{F129S} expression construct; and injecting into the inflamed joint of the subject a second dose of the composition 240 days after injection of the first dose, wherein the second dose of the composition comprises 450 μg of the IL-10^{F129S} expression construct. In some embodiments, the osteoarthritis is osteoarthritis of the knee.

In certain embodiments, the present disclosure provides a method for treating an inflammatory joint disease in a subject, comprising injecting into an inflamed joint of the subject a first dose of a composition comprising an IL-10^{F129S} expression construct, wherein the first dose of the composition comprises 450 μg of the IL-10^{F129S} expression construct; and injecting into the inflamed joint of the subject a second dose of the composition 150-360 days after injection of the first dose, wherein the second dose of the composition comprises 450 μg of the IL-10^{F129S} expression construct, and wherein the first dose and the second dose of the composition are formulated in a solution of phosphate-buffered saline (pH 7.4) with 3% w/v sucrose and 2.5 mg/mL D-mannose. In certain embodiments, the present disclosure provides a method for treating an inflammatory joint disease in a subject, comprising injecting into an inflamed joint of the subject a first dose of a composition comprising an IL-10^{F129S} expression construct, wherein the first dose of the composition comprises 450 μg of the IL-10^{F129S} expression construct; and injecting into the inflamed joint of the subject a second dose of the composition 120 days after injection of the first dose, wherein the second dose of the composition comprises 450 μg of the IL-10^{F129S} expression construct, and wherein the first dose and the second dose of the composition are formulated in a solution of phosphate-buffered saline (pH 7.4) with 3% w/v sucrose and 2.5 mg/mL D-mannose. In certain embodiments, the present disclosure provides a method for treating an inflammatory joint disease in a subject, comprising injecting into an inflamed joint of the subject a first dose of a composition comprising an IL-10^{F129S} expression construct, wherein the first dose of the composition comprises 450 μg of the IL-10^{F129S} expression construct; and injecting into the inflamed joint of the subject a second dose of the composition 150 days after injection of the first dose, wherein the second dose of the composition comprises 450 μg of the IL-10^{F129S} expression construct, and wherein the first dose and the second dose of the composition are formulated in a solution of phosphate-buffered saline (pH 7.4) with 3% w/v sucrose and 2.5 mg/mL D-mannose. In certain embodiments, the present disclosure provides a method for treating an inflammatory joint disease in a subject, comprising injecting into an inflamed joint of the subject a first dose of a composition comprising an IL-10^{F129S} expression construct, wherein the first dose of the composition comprises 450 μg of the IL-10^{F129S} expression construct; and injecting into the inflamed joint of the subject a second dose of the composition 180 days after injection of the first dose, wherein the second dose of the composition comprises 450 μg of the IL-10^{F129S} expression construct, and wherein the first dose and the second dose of the composition are formulated in a solution of phosphate-buffered saline (pH 7.4) with 3% w/v sucrose and 2.5 mg/mL D-mannose. In certain embodiments, the present disclosure provides a method for treating an inflammatory joint disease in a subject, comprising injecting into an inflamed joint of the subject a first dose of a composition comprising an IL-10^{F129S} expression construct, wherein the first dose of the composition comprises 450 μg of the IL-10^{F129S} expression construct; and injecting into the inflamed joint of the subject a second dose of the composition 210 days after injection of the first dose, wherein the second dose of the composition comprises 450 μg of the IL-10^{F129S} expression construct, and wherein the first dose and the second dose of the composition are formulated in a solution of phosphate-buffered saline (pH 7.4) with 3% w/v sucrose and 2.5 mg/mL D-mannose. In certain embodiments, the present disclosure provides a method for treating an inflammatory joint disease in a subject, comprising injecting into an inflamed joint of the subject a first dose of a composition comprising an IL-10^{F129S} expression construct, wherein the first dose of the composition comprises 450 μg of the IL-10^{F129S} expression construct; and injecting into the inflamed joint of the subject a second dose of the composition 240 days after injection of the first dose, wherein the second dose of the composition comprises 450 μg of the IL-10^{F129S} expression construct, and wherein the first dose and the second dose of the composition are formulated in a solution of phosphate-buffered saline (pH 7.4) with 3% w/v sucrose and 2.5 mg/mL D-mannose. In certain embodiments, the present disclosure provides a method for treating an inflammatory joint disease in a subject, comprising injecting into an inflamed joint of the subject a first dose of a composition comprising an IL-10^{F129S} expression construct, wherein the first dose of the composition comprises 450 μg of the IL-10^{F129S} expression construct; and injecting into the inflamed joint of the subject a second dose of the composition 270 days after injection of the first dose, wherein the second dose of the composition comprises 450 μg of the IL-10^{F129S} expression construct, and wherein the first dose and the second dose of the composition are formulated in a solution of phosphate-buffered saline (pH 7.4) with 3% w/v sucrose and 2.5 mg/mL D-mannose.

In some embodiments, the subject in which an inflammatory joint disease is treated is a mammal. In certain embodiments, the subject is a non-human animal (e.g., primate, cattle, pig, horse, sheep, goat, camel, cat, or dog). In certain embodiments, the subject is a human, horse, dog, or cat. In certain embodiments, the subject is a human, horse, or dog. In certain embodiments, the subject is a human. A subject who may benefit from treatment using the methods described herein may be selected based on various criteria. In some embodiments, the subject has been diagnosed with osteoarthritis. In some embodiments, the subject is an osteoarthritis of the knee patient with a WOMAC score of greater than 9 (indicating moderate-to-severe disease). In some embodiments, the subject is an osteoarthritis patient with a WOMAC score of greater than 9 (indicating moderate-to-severe disease). In some embodiments, the subject is an osteoarthritis patient with a Kellgren-Lawrence (KL) grade of 1-4 (e.g., 2 or 3) (i.e., KL3 is Kellgren-Lawrence grade 3). In some embodiments, the subject benefits from treatment of the inflammatory joint disease for at least one year from the injection of the first dose. In some embodiments, the subject benefits from treatment of the inflammatory joint disease for at least half of a year from the injection of the first dose. In some embodiments, the subject benefits from treatment of the inflammatory joint disease for at least 4 months from the injection of the first dose. In some embodiments, the subject benefits from treatment of the inflammatory joint disease for at least 3 months from the injection of the first dose. In some embodiments, the composition injected in the first and the second dose has an excellent safety profile. In some embodiments, the composition administered in the first and the second dose result in no drug related serious adverse events. In some embodiments, the subject has no immune response following injection of the composition. In certain embodiments, the subject has minimal or no antibody production to IL-10 following injection of the composition (i.e., after 180 or more days (e.g., 360 days)). In some embodiments, the subject has no anti-IL-10 antibodies within 360 days following injection of the composition.

The present disclosure also contemplates use of a composition comprising an IL-10^{F129S} expression construct for the treatment of an inflammatory joint disease, wherein the composition is for administration in a first dose comprising 100 μg to 600 μg of the IL-10^{F129S} expression construct, and wherein the composition is for administration in a second dose comprising 100 μg to 600 μg of the IL-10^{F129S} expression construct 90-30 days after the first dose. The present disclosure also contemplates use of a composition comprising an IL-10^{F129S} expression construct for the treatment of an inflammatory joint disease, wherein the composition is for administration in a first dose comprising 100 μg to 600 μg of the IL-10^{F129S} expression construct, and wherein the composition is for administration in a second dose comprising 100 μg to 600 μg of the IL-10^{F129S} expression construct 90-330 days after the first dose.

The present disclosure also contemplates use of a composition comprising an IL-10^{F129S} expression construct for the treatment of an inflammatory joint disease, wherein the composition is for administration in a first dose comprising 100 μg to 600 μg of the IL-10^{F129S} expression construct, and wherein the composition is for administration in a second dose comprising 100 μg to 600 μg of the IL-10^{F129S} expression construct 120-30 days after the first dose. The present disclosure also contemplates use of a composition comprising an IL-10^{F129S} expression construct for the treatment of an inflammatory joint disease, wherein the composition is for administration in a first dose comprising 100 μg to 600 μg of the IL-10^{F129S} expression construct, and wherein the composition is for administration in a second dose comprising 100 μg to 600 μg of the IL-10^{F129S} expression construct 120-330 days after the first dose.

The present disclosure also contemplates use of a composition comprising an IL-10^{F129S} expression construct for the treatment of an inflammatory joint disease, wherein the composition is for administration in a first dose comprising 100 μg to 600 μg of the IL-10^{F129S} expression construct, and wherein the composition is for administration in a second dose comprising 100 μg to 600 μg of the IL-10^{F129S} expression construct 180-30 days after the first dose. The present disclosure also contemplates use of a composition comprising an IL-10^{F129S} expression construct for the treatment of an inflammatory joint disease, wherein the composition is for administration in a first dose comprising 100 μg to 600 μg of the IL-10^{F129S} expression construct, and wherein the composition is for administration in a second dose comprising 100 μg to 600 μg of the IL-10^{F129S} expression construct 180-330 days after the first dose.

### Kits

Also encompassed by the disclosure are kits (e.g., pharmaceutical packs). The kits provided may comprise a first dose and a second dose of a pharmaceutical composition comprising an IL-10^{F129S} expression construct as described herein and a container (e.g., a vial, ampule, bottle, syringe, and/or dispenser package, or other suitable container). Also provided herein are kits comprising a first dose and a second dose of an IL-10F129S expression construct, wherein the first dose comprises 150 μg or 450 μg of the expression construct, and wherein the second dose comprises 450 μg of the expression construct; and wherein the first and/or the second dose of the composition is formulated in a sterile solution of phosphate-buffered saline (pH 7.4) with about 3% w/v sucrose and about 2.5 mg/mL D-mannose. In some embodiments, the provided kits may optionally further include a second container comprising a pharmaceutical excipient for dilution or suspension of a composition (e.g., phosphate-buffered saline). In some embodiments, the composition provided in the first container and the pharmaceutical excipient provided in the second container are combined to form one unit dosage form.

Thus, in one aspect, the present disclosure provides kits including a first dose and a second dose of an IL-10^{F129S} expression construct. In some embodiments, the first dose comprises 100 μg to 600 μg of the expression construct, and the second dose comprises 100 μg to 600 μg of the expression construct. In certain embodiments, the first dose comprises about 450 μg of the expression construct. In certain embodiments, the second dose comprises about 450 μg of the expression construct. In some embodiments, the first dose comprises 450 μg of the expression construct and the second dose comprises 450 μg of the expression construct. In some embodiments, the first and/or the second dose of the composition is formulated in a sterile solution of phosphate-buffered saline (pH 7.4) with about 3% w/v sucrose and about 2.5 mg/mL D-mannose. In some embodiments, the first and/or the second dose of the composition comprises 0.45 mg/mL of vector and is formulated in a sterile solution of phosphate-buffered saline (pH 7.4) with about 3% w/v sucrose and about 2.5 mg/mL D-mannose. In some embodiments, the first and/or the second dose of the composition is formulated in a sterile solution of phosphate-buffered saline (pH 7.4) with about 3% w/v sucrose and about 2.5 mg/mL D-mannose. In some embodiments, the first and/or the second dose of the composition comprises 0.1-0.6 mg/mL of vector and is formulated in a sterile solution of phosphate-buffered saline (pH 7.4) with about 3% w/v sucrose and about 2.5 mg/mL D-mannose. In some embodiments, the first and/or the second dose of the composition comprises 0.15-0.45 mg/mL of vector and is formulated in a sterile solution of phosphate-buffered saline (pH 7.4) with about 3% w/v sucrose and about 2.5 mg/mL D-mannose. In some embodiments, the first and/or the second dose of the composition comprises 0.15 mg/mL of vector and is formulated in a sterile solution of phosphate-buffered saline (pH 7.4) with about 3% w/v sucrose and about 2.5 mg/mL D-mannose. The volume of the injected composition depends on the joint. In some embodiments, the injected composition is approximately 0.5-5 mL in volume. In some embodiments, the injected composition is approximately 1 mL in volume. In certain embodiments, the kits are useful for treating a disease (e.g., an inflammatory joint disease) in a subject in need thereof. In certain embodiments, the kits are useful for preventing a disease (e.g., an inflammatory joint disease) in a subject in need thereof. In certain embodiments, the kits are useful for reducing the risk of developing a disease (e.g., an inflammatory joint disease, such as osteoarthritis) in a subject in need thereof.

In certain embodiments, a kit described herein further includes instructions for using the kit. A kit described herein may also include information as required by a regulatory agency, such as the U.S. Food and Drug Administration (FDA). In certain embodiments, the information included in the kits is prescribing information. In certain embodiments, the kits and instructions provide for treating a disease (e.g., an inflammatory joint disease, such as osteoarthritis) in a subject in need thereof. In certain embodiments, the kits and instructions provide for preventing a disease (e.g., an inflammatory joint disease) in a subject in need thereof. In certain embodiments, the kits and instructions provide for reducing the risk of developing a disease (e.g., an inflammatory joint disease, such as osteoarthritis) in a subject in need thereof. A kit described herein may include one or more additional pharmaceutical agents described herein as a separate composition.

### Additional Embodiments

1. A method for treating an inflammatory joint disease in a subject, the method comprising:
injecting into an inflamed joint of the subject a first dose of a composition comprising an IL-10^{F129S} expression construct, wherein the first dose of the composition comprises 100 μg to 600 μg of the IL-10^{F129S} expression construct; and
injecting into the inflamed joint of the subject a second dose of the composition 90-330 days after injection of the first dose, wherein the second dose of the composition comprises 100 μg to 600 μg of the IL-10^{F129S} expression construct.

2. The method of embodiment 1, wherein the second dose of the composition is injected about 90-270 days, about 100-260 days, about 110-250 days, about 120-240 days, about 130-230 days, about 140-220 days, about 150-210 days, about 160-200 days, or about 170-190 days after injection of the first dose.

3. The method of embodiment 1, wherein the second dose of the composition is injected about 100-330 days, about 110-330 days, about 120-330 days, about 130-330 days, about 140-330 days, about 150-330 days, about 160-330 days, about 170-330 days, or about 180-330 days after injection of the first dose.

4. The method of embodiment 1, wherein the second dose of the composition is injected 180 or more days after injection of the first dose.

5. The method of embodiment 1, wherein the second dose of the composition is injected about 180-330 days, about 180-320 days, about 180-310 days, about 180-300 days, about 180-290 days, about 180-280 days, about 180-270 days, about 180-260 days, about 180-250 days, about 180-240 days, about 180-230 days, about 180-220 days, about 180-210 days, about 180-200 days, or about 180-190 days after injection of the first dose.

6. The method of embodiment 1, wherein the second dose of the composition is injected about 170-190 days after injection of the first dose.

7. The method of any one of embodiments 1-6, wherein the second dose of the composition is injected 180 days after injection of the first dose.

8. The method of any one of embodiments 1-7, wherein the first dose comprises 100 μg to 200 μg, 110 μg to 190 μg, 120 μg to 180 μg, 130 μg to 170 μg, or 140 μg to 160 μg of the IL-10^{F129S} expression construct.

9. The method of any one of embodiments 1-8, wherein the first dose comprises about 150 μg of the IL-10^{F129S} expression construct.

10. The method of any one of embodiments 1-7, wherein the first dose comprises 400 μg to 500 μg, 410 μg to 490 μg, 420 μg to 480 μg, 430 μg to 470 μg, or 440 μg to 460 μg of the IL-10^{F129S} expression construct.

11. The method of any one of embodiments 1-7 or 10, wherein the first dose comprises about 450 μg of the IL-10^{F129S} expression construct.

12. The method of any one of embodiments 1-11, wherein the second dose comprises 400 μg to 500 μg, 410 μg to 490 μg, 420 μg to 480 μg, 430 μg to 470 μg, or 440 μg to 460 μg of the IL-10^{F129S} expression construct.

13. The method of any one of embodiments 1-12, wherein the second dose comprises about 450 μg of the IL-10^{F129S} expression construct.

14. The method of any one of embodiments 1-7, wherein the first dose comprises about 150 μg of the IL-10^{F129S} expression construct, and the second dose comprises about 450 μg of the IL-10^{F129S} expression construct.

15. The method of any one of embodiments 1-7, wherein the first dose comprises about 450 μg of the IL-10^{F129S} expression construct, and the second dose comprises about 450 μg of the IL-10^{F129S} expression construct.

16. The method of embodiment 15, wherein the second dose of the composition is injected 180 days after injection of the first dose.

17. The method of any one of embodiments 1-16, wherein treating the subject with a first dose and a second dose results in increased efficacy compared to a method in which a subject is treated with only the first dose.

18. The method of any one of embodiments 1-17, wherein the IL-10^{F129S} expression construct comprises a plasmid backbone.

19. The method of embodiment 18, wherein the plasmid backbone comprises a bacterial or viral backbone.

20. The method of any one of embodiments 1-19, wherein the IL-10^{F129S} expression construct comprises a nucleic acid sequence encoding IL-10^{F129S}.

21. The method of embodiment 20, wherein the IL-10^{F129S} expression construct comprises adeno-associated virus (AAV) inverted terminal repeat (ITR) sequences flanking the nucleic acid sequence encoding interleukin-10^{F129S}.

22. The method of any one of embodiments 1-21, wherein the IL-10^{F129S} expression construct comprises one or more promoter sequences.

23. The method of embodiment 22, wherein the one or more promoter sequences are each independently selected from the group consisting of chicken or human β-actin promoters, cytomegalovirus immediate early promoters, glyceraldehyde 3-phosphate dehydrogenase (GADPH) promoters, elongation factor 1α (eF1α) promoters, GFAP promoters, murine leukemia virus (MLV) promoters, herpes simplex virus thymidine kinase (TK) promoters, and woodchuck hepatitis virus post-transcriptional regulatory element (WPRE) promoters.

24. The method of embodiment 22 or 23, wherein the IL-10^{F129S} expression construct comprises a cytomegalovirus immediate early promoter.

25. The method of any one of embodiments 1-24, wherein the IL-10^{F129S} expression construct comprises a polyadenylation signal operably linked to the nucleic acid sequence encoding IL-10^{F129S}.

26. The method of embodiment 25, wherein the polyadenylation signal is selected from the group consisting of an SV40 polyadenylation signal, a bovine growth hormone polyadenylation signal, and a synthetic polyadenylation signal.

27. The method of embodiment 25 or 26, wherein the polyadenylation signal is an SV40 polyadenylation signal.

28. The method of any one of embodiments 1-27, wherein the IL-10^{F129S} expression construct comprises a marker gene.

29. The method of embodiment 28, wherein the marker gene is selected from the group consisting of a neomycin resistance marker, an ampicillin resistance marker, and a kanamycin resistance marker.

30. The method of embodiment 28 or 29, wherein the marker gene is a kanamycin resistance marker.

31. The method of any one of embodiments 1-30, wherein the IL-10^{F129S} expression construct comprises one or more nuclear targeting sequences.

32. The method of embodiment 31, wherein the nuclear targeting sequence is 5′ to the nucleic acid sequence encoding interleukin-10^{F129S}.

33. The method of embodiment 31, wherein the nuclear targeting sequence is 3′ to the nucleic acid sequence encoding interleukin-10^{F129S}.

34. The method of embodiment 31, wherein the IL-10^{F129S} expression construct comprises one or more nuclear targeting sequences 5′ to the nucleic acid sequence encoding interleukin-10^{F129S} and one or more nuclear targeting sequences 3′ to the nucleic acid sequence encoding interleukin-10^{F129S}.

35. The method of any one of embodiments 1-34, wherein the IL-10^{F129S} expression construct comprises one or more control sequences operably linked to the nucleic acid sequence encoding IL-10^{F129S}.

36. The method of embodiment 35, wherein the one or more control sequences are each independently selected from the group consisting of transcription termination sequences, upstream regulatory domains, and internal ribosome entry sites.

37. The method of embodiment 1, wherein the construct comprises:
two AAV ITRs;
a chicken β-actin promoter, a herpes simplex virus thymidine kinase (TK) promoter, an SV40 polyadenylation signal, and a cytomegalovirus immediate early promoter enhancer; and
a neomycin resistance marker.

38. The method of embodiment 1, wherein the construct comprises:
two AAV ITRs;
a cytomegalovirus immediate early promoter, an SV40 polyadenylation signal, and a cytomegalovirus immediate early promoter enhancer; and
an ampicillin resistance marker.

39. The method of embodiment 1, wherein the construct comprises:
two AAV ITRs;
a cytomegalovirus immediate early promoter, an SV40 polyadenylation signal, and a cytomegalovirus immediate early promoter enhancer; and
a kanamycin resistance marker.

40. The method of any one of embodiments 1-39, wherein the expression construct comprises a sequence that is at least 80%, at least 85%, at least 90%, at least 95%, or at least 99% identical to the nucleotide sequence of SEQ ID NO: 1.

41. The method of any one of embodiments 1-40, wherein the expression construct consists of the nucleotide sequence of SEQ ID NO: 1.

42. The method of any one of embodiments 1-41, wherein the first dose of the composition and/or the second dose of the composition is administered with an excipient that increases the uptake of the expression construct.

43. The method of embodiment 42, wherein the excipient that increases the uptake of the expression construct is selected from the group consisting of a sugar, calcium phosphate, a dendrimer, an oligonucleotide, a high molecular weight hyaluronic acid, and a lipid.

44. The method of embodiment 43, wherein the sugar is D-mannose, sucrose, or glucose.

45. The method of embodiment 43 or 44, wherein the sugar is D-mannose.

46. The method of any one of embodiments 42-45, wherein the excipient that increases the uptake of the expression construct is administered concurrently with the composition.

47. The method of any one of embodiments 1-46, wherein the composition further comprises a diluent.

48. The method of embodiment 47, wherein the diluent is phosphate-buffered saline (pH 7.4).

49. The method of any one of embodiments 1-48, wherein the inflamed joint is a knee, an elbow, a wrist, an ankle, a hip, a shoulder, a jaw, or a spine.

50. The method of embodiment 49, wherein the inflamed joint is a knee.

51. The method of any one of embodiments 1-50, wherein the inflammatory joint disease is arthritis, tendonitis, bursitis, inflammation of the ligament, synovitis, gout, facet syndrome, or systemic lupus erythematosus.

52. The method of embodiment 51, wherein the arthritis is rheumatoid arthritis.

53. The method of embodiment 51, wherein the arthritis is osteoarthritis.

54. The method of any one of embodiments 1-53, wherein the first and/or the second dose of the composition is formulated in a solution of phosphate-buffered saline with sucrose and D-mannose.

55. The method of embodiment 54, wherein the composition comprises about 3% w/v sucrose.

56. The method of embodiment 54 or 55, wherein the composition comprises about 2.5 mg/mL D-mannose.

57. The method of any one of embodiments 1-56, wherein the first and the second dose of the composition are formulated in a solution of phosphate-buffered saline (pH 7.4) with 3% w/v sucrose and 2.5 mg-mL D-mannose.

58. A method for treating an inflammatory joint disease in a subject, comprising:
injecting into an inflamed joint of the subject a first dose of a composition comprising an IL-10^{F129S} expression construct, wherein the first dose of the composition comprises 450 μg of the IL-10^{F129S} expression construct; and
injecting into the inflamed joint of the subject a second dose of the composition 180 days after injection of the first dose, wherein the second dose of the composition comprises 450 μg of the IL-10^{F129S} expression construct.

59. The method of embodiment 58, wherein the first and the second dose of the composition are formulated in a solution of phosphate-buffered saline (pH 7.4) with 3% w/v sucrose and 2.5 mg-mL D-mannose.

60. The method of any one of embodiments 1-59, wherein the subject is a mammal.

61. The method of any one of embodiments 1-60, wherein the subject is a human.

62. The method of any one of embodiments 1-60, wherein the subject is a primate, cattle, pig, horse, sheep, goat, camel, cat, or dog.

63. The method of any one of embodiments 1-62, wherein the subject is an osteoarthritis patient with a WOMAC score of greater than 9.

64. The method of embodiment 63, wherein a WOMAC score of greater than 9 indicates that the subject has moderate-to-severe osteoarthritis.

65. The method of any one of embodiments 1-64, wherein the subject is an osteoarthritis patient with a Kellgren-Lawrence (KL) grade of 1-4.

66. The method of any one of embodiments 1-65, wherein the subject benefits from treatment of the inflammatory joint disease for at least one year from the injection of the first dose.

67. The method of any one of embodiments 1-66, wherein the composition administered in the first and the second dose has an excellent safety profile.

68. The method of any one of embodiments 1-67, wherein the subject has minimal antibody production to IL-10 following injection of the composition.

69. Use of a composition comprising an IL-10^{F129S} expression construct for the treatment of an inflammatory joint disease, wherein the composition is for administration in a first dose comprising 100 μg to 600 μg of the IL-10^{F129S} expression construct, and wherein the composition is for administration in a second dose comprising 100 μg to 600 μg of the IL-10^{F129S} expression construct 90-330 days after the first dose.

70. A kit comprising a first dose and a second dose of an IL-10^{F129S} expression construct, wherein the first dose comprises 100 μg to 600 μg of the expression construct, and wherein the second dose comprises 100 μg to 600 μg of the expression construct.

71. The kit of embodiment 70, wherein the first dose comprises about 450 μg of the expression construct.

72. The kit of embodiment 70 or 71, wherein the second dose comprises about 450 μg of the expression construct.

73. The kit of any one of embodiments 70-72, wherein the first and/or the second dose of the composition is formulated in a sterile solution of phosphate-buffered saline (pH 7.4) with about 3% w/v sucrose and about 2.5 mg/mL D-mannose.

### EXAMPLES

### Example 1: Clinical Studies of Treatment of Knee Osteoarthritis

A randomized, double-blind, placebo-controlled Phase 2 study (study 204) of XT-150 was conducted in patients with moderate-to-severe pain due to osteoarthritis of the knee. Stage A of this study comprised a placebo-controlled efficacy assessment at six months, and stage B comprised an additional six-month safety follow-up (with an opportunity for second injection to the knee previously injected). The objectives of this study included 1) establishing safety of XT-150 with single (stage A) and repeat (stage B) dosing; 2) evaluating efficacy of XT-150 in a broader patient population with moderate to severe pain; 3) determining the preferred dose by comparing 150 μg and 450 μg efficacy to placebo; and 4) evaluating the benefit of repeat dosing.

Insights from this study confirmed that XT-150 continues to show the safety profile seen in previous studies, with no drug related serious adverse events in 286 patients dosed with either the single or a repeat dose. Interim top-line results did not distinguish XT-150 single-dose from placebo in 6-month endpoint as measured by 30% responder rates in expanded osteoarthritis patient severity population. A single dose of XT-150 at 450 μg showed pain reduction, separating from placebo, in a more severe osteoarthritis targeted patient population with a baseline WOMAC pain score of 9-14, KL3 as measured by 50% responder rates. Double-dosed patients (including those administered a 150 μg first dose and a 150 μg second dose, a 150 μg first dose and a 450 μg second dose, a 450 μg first dose and a 150 μg second dose, or a 450 μg first dose and a 450 μg second dose) targeted at 9-14 WOMAC pain score and KL2/3 pain scores showed statistically significant differences from single dose treatment as measured by 50% responder rates. Double doses of 450 μg differentiated statistically over a single injection of 450 μg. Enhanced improvement in WOMAC function scores were also observed in repeat dosing with statistically significant differentiation from single dose. Finally, a meaningful benefit of repeat dosing with 450 μg of XT-150 was observed across a broader osteoarthritis severity population (≥9 WOMAC pain score). More than half of the targeted patients achieved a substantial ≥50% pain reduction. XT-150 therefore offers a safe and transformational therapy for sustained pain reduction benefit of at least 360 days.

Overall, these studies showed that a single dose of XT-150 at 450 μg results in pain reduction separating from placebo for more severe osteoarthritis patients. A meaningful benefit of repeat dosing (e.g., two 450 μg doses spaced, for example, approximately 180 days apart) was also observed across a broader osteoarthritis severity population (i.e., wider starting WOMAC scores). Targeted patients, with a baseline WOMAC 9-14, receiving two injections of XT-150 showed statistically significant improvement in 50% responder rates compared for WOMAC pain to a single XT-150 dose. Enhanced improvement in WOMAC function scores were also observed with repeat dosing with statistical significance. Finally, XT-150 shows a good safety profile, and no drug related serious adverse events were observed to date in 286 patients dosed.

### Example 2: Clinical Studies of Treatment of Knee Osteoarthritis

A subsequent trial was designed to allow a more complete evaluation of the safety and efficacy of repeat dosing. In Stage A, 150 μg and 450 μg single injection cohorts were studied versus placebo for 6 months; and in Stage B, subjects in all three cohorts had the option for a second injection, pre-randomized to either 150 μg or 450 μg, administered at the subject's request from Day 180 to Day 330. Subjects and investigators remained blinded to who was on placebo/150 μg/450 μg for stage A, and to which dose they would receive in Stage B throughout the entire 360 days of the study. Unlike other pain medications, XT-150 is not a short-acting analgesic, but rather is ‘disease-altering’ at the site of inflammation. The sustained release of IL-10 seeks to restore cytokine homeostasis and inflammatory balance in the knee. This mechanism of action delivers a long-lasting benefit to patients, and thus it sought to determine whether a second dose maintained or improved efficacy from the first dose. Stage B was designed where patients could elect a second injection at any time from Day 180 to Day 330 of the trial. While this option was elective, it is important to emphasize that all patients at Day 0 were pre-randomized to receive either 150 μg or 450 μg doses if they elected the second dose. The vast majority of patients (>85%) elected to receive active dose in Stage B. Site investigators and patients were completely blind to which six cohorts patients were assigned to. In other words, they did not know if patients were in the single active injection vs repeat active injection group. In addition, all patients were allowed to maintain their existing stable pain medication regimen to mimic a real-world situation where patients, given their severity, would likely be on a number of additional pain medications. As such, any efficacy and safety data observed is on top of existing medications with no washout period prior to starting in the study. To assist with patient recruitment and retention, it was determined not to maintain the original placebo subjects for one year on placebo in this study. Hence, all injections in Part B were with XT-150 so anyone in the study was guaranteed to receive at least one dose of XT-150.

A total of 55 patients received XT-150 at doses from 15 μg to 600 μg; this includes 44 patients who received a single dose and 11 patients who received a second dose. There were no drug related serious adverse events (SAEs). The only drug-related adverse event were transient, mild-moderate discomfort in the knee following injection seen in 8 patients. No treatment related changes were detected in CBC, coagulation, metabolic profile, or acute phase biomarkers. XT-150 plasmid DNA could be detected by qPCR in blood specimens drawn 4 hours after intra-articular injection. There were 2 patients with detectable XT-150 plasmid DNA at Day 7, and no plasmid DNA detected was detected at Day 14 in the blood. Anti-IL-10 antibodies were not detected following single or repeat doses. Serum IL-10 levels were low, at physiologic levels, and were not related to dose or time following XT-150 injection. The highest IL-10 level detected was 277.4 μg/mL at baseline in a patient receiving placebo and declined to 182.5 μg/mL at Day 180.

For patients with WOMAC 9-20, the difference between a single vs. repeat dose is statically significant, suggesting that the target patient population and screening threshold for future studies should be consistent WOMAC pain >9 (moderate to severe pain definition) at screening and baseline.

Based on the safety evaluations conducted, no safety concerns were identified for XT-150 when administered as a single or multiple injections in the knee at doses of 150 μg or 450 μg in adult participants with osteoarthritic pain of the knee. The 18 SAEs were considered unrelated to the study treatment. Only 6 treatment emergent adverse events (TEAE) were deemed related to study treatment across the entire study. This included 3 events of arthralgia (1 mild, 2 moderate), one event of injection site swelling (moderate), one event of injection site pain (mild) and one event of injection site warmth (moderate). Adverse events of special interest (AESI) reported in this study included nausea, headache, back pain, influenza like illness, and pyrexia. None of the AESIs were deemed related to study treatment. There were no AESIs arising from abnormal clinical laboratory assessments. There were no differences in the incidence of abnormal (clinically significant) injection site examinations across treatments. There was no incidence of anti-IL-10 antibodies in any participant throughout the study. There were no clear differences in safety as assessed by clinical laboratory tests, physical examinations, or vital signs, between participants following XT-150 150 μg, XT-150 450 μg or placebo, no dose-related trends in participants who received XT-150.

The study confirmed the safety of two (2) doses of XT-150 and provided insights and findings with regards to efficacy. The data support XT-150 as a potential transformative therapy for patients with moderate to severe OA of the knee.

### EQUIVALENTS AND SCOPE

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permit the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A method for treating an inflammatory joint disease in a subject, the method comprising:
injecting into an inflamed joint of the subject a first dose of a composition comprising an IL-10^{F129S} expression construct, wherein the first dose of the composition comprises 100 μg to 600 μg of the IL-10^{F129S} expression construct; and
injecting into the inflamed joint of the subject a second dose of the composition 150-360 days after injection of the first dose, wherein the second dose of the composition comprises 100 μg to 600 μg of the IL-10^{F129S} expression construct.

2. The method of claim 1, wherein the second dose of the composition is injected about 150-270 days, about 150-260 days, about 150-250 days, about 150-240 days, about 150-230 days, about 150-220 days, about 150-210 days, about 160-200 days, or about 170-190 days after injection of the first dose.

3. The method of claim 1, wherein the second dose of the composition is injected about 150-330 days, about 150-330 days, about 150-330 days, about 105-330 days, about 140-330 days, about 150-330 days, about 160-330 days, about 170-330 days, or about 180-330 days after injection of the first dose.

4. The method of claim 1, wherein the second dose of the composition is injected 150 or more days after injection of the first dose.

5. The method of claim 1, wherein the second dose of the composition is injected about 150 days after injection of the first dose.

6. The method of claim 1, wherein the second dose of the composition is injected 180 or more days after injection of the first dose.

7. The method of claim 1, wherein the second dose of the composition is injected about 180 days after injection of the first dose.

8. The method of any one of claims 1-7, wherein the first dose comprises 100 μg to 200 μg, 110 μg to 190 μg, 120 μg to 180 μg, 130 μg to 170 μg, or 140 μg to 160 μg of the IL-10^{F129S} expression construct.

9. The method of any one of claims 1-8, wherein the first dose comprises about 150 μg of the IL-10^{F129S} expression construct.

10. The method of any one of claims 1-7, wherein the first dose comprises 400 μg to 500 μg, 410 μg to 490 μg, 420 μg to 480 μg, 430 μg to 470 μg, or 440 μg to 460 μg of the IL-10^{F129S} expression construct.

11. The method of any one of claims 1-7 or 10, wherein the first dose comprises about 450 μg of the IL-10^{F129S} expression construct.

12. The method of any one of claims 1-11, wherein the second dose comprises 400 μg to 500 μg, 410 μg to 490 μg, 420 μg to 480 μg, 430 μg to 470 μg, or 440 μg to 460 μg of the IL-10^{F129S} expression construct.

13. The method of any one of claims 1-12, wherein the second dose comprises about 450 μg of the IL-10^{F129S} expression construct.

14. The method of any one of claims 1-7, wherein the first dose comprises about 150 μg of the IL-10^{F129S} expression construct, and the second dose comprises about 450 μg of the IL-10^{F129S} expression construct.

15. The method of any one of claims 1-7, wherein the first dose comprises about 450 μg of the IL-10^{F129S} expression construct, and the second dose comprises about 450 μg of the IL-10^{F129S} expression construct.

16. The method of claim 14 or 15, wherein the second dose of the composition is injected about 150-210 days after injection of the first dose.

17. A method for treating an inflammatory joint disease in a subject, comprising:
injecting into an inflamed joint of the subject a first dose of a composition comprising an IL-10^{F129S} expression construct, wherein the first dose of the composition comprises 450 μg of the IL-10^{F129S} expression construct; and
injecting into the inflamed joint of the subject a second dose of the composition about 150-180 days after injection of the first dose, wherein the second dose of the composition comprises 450 μg of the IL-10^{F129S} expression construct.

18. The method of any one of claims 1-17, wherein treating the subject with a first dose and a second dose results in increased efficacy compared to a method in which a subject is treated with only the first dose.

19. The method of any one of claims 1-18, wherein the construct comprises:
two AAV ITRs;
a chicken β-actin promoter, a herpes simplex virus thymidine kinase (TK) promoter, an SV40 polyadenylation signal, and a cytomegalovirus immediate early promoter enhancer; and
an antibiotic resistance marker.

20. The method of any one of claims 1-19, wherein the expression construct comprises a sequence that is at least 80%, at least 85%, at least 90%, at least 95%, or at least 99% identical to the nucleotide sequence of SEQ ID NO: 1.

21. The method of any one of claims 1-20, wherein the expression construct consists of the nucleotide sequence of SEQ ID NO: 1.

22. The method of any one of claims 1-21, wherein the first dose of the composition and/or the second dose of the composition further comprises one or more excipients.

23. The method of claim 22, wherein the one or more excipients increase the uptake of the expression construct.

24. The method of claim 22 or 23, wherein the one or more excipients are selected from the group consisting of a sugar, calcium phosphate, a dendrimer, an oligonucleotide, a high molecular weight hyaluronic acid, and a lipid.

25. The method of claim 24, wherein the sugar is mannose, sucrose, or glucose.

26. The method of claim 24 or 25, wherein the sugar is D-mannose.

27. The method of claim 24 or 25, wherein the sugar is sucrose.

28. The method of claim 22 or 23, wherein the one or more excipients are sugar and D-mannose.

29. The method of any one of claims 1-28, wherein the composition further comprises a diluent.

30. The method of claim 29, wherein the diluent is phosphate-buffered saline (pH 7.4).

31. The method of any one of claims 1-30, wherein the first and/or the second dose of the composition further comprises phosphate-buffered saline, sucrose, and D-mannose.

32. The method of claim 31, wherein the composition comprises about 3% w/v sucrose.

33. The method of claim 31 or 32, wherein the composition comprises about 2.5 mg/mL D-mannose.

34. The method of any one of claims 1-33, wherein the first and the second dose of the composition are formulated in a solution of phosphate-buffered saline (pH 7.4) with 3% w/v sucrose and 2.5 mg-mL D-mannose.

35. The method of claim any one of claims 1-34, wherein the composition comprises about 0.15-0.45 mg/mL of IL-10^{F129S} expression construct.

36. The method of claim any one of claims 1-35, wherein the composition comprises about 0.15 mg/mL of IL-10^{F129S} expression construct.

37. The method of claim any one of claims 1-35, wherein the composition comprises about 0.45 mg/mL of IL-10^{F129S} expression construct.

38. The method of any one of claims 1-37, wherein the inflamed joint is a knee, an elbow, a wrist, an ankle, a hip, a shoulder, a jaw, or a spine.

39. The method of claim 38, wherein the inflamed joint is a knee.

40. The method of any one of claims 1-39, wherein the inflammatory joint disease is arthritis, tendonitis, bursitis, inflammation of the ligament, synovitis, gout, facet syndrome, or systemic lupus erythematosus.

41. The method of claim 40, wherein the arthritis is rheumatoid arthritis.

42. The method of claim 40, wherein the arthritis is osteoarthritis.

43. The method of claim 40, wherein the arthritis is osteoarthritis of the knee.

44. The method of any one of claims 1-43, wherein the subject is a mammal.

45. The method of any one of claims 1-44, wherein the subject is a human.

46. The method of any one of claims 1-45, wherein the subject is a primate, cattle, pig, horse, sheep, goat, camel, cat, or dog.

47. The method of any one of claims 1-46, wherein the subject is an osteoarthritis patient with a WOMAC score of greater than 9.

48. The method of any one of claims 1-47, wherein the subject is an osteoarthritis of the knee patient with a WOMAC score of greater than 9.

49. The method of any one of claims 1-48, wherein the subject is an osteoarthritis patient with a Kellgren-Lawrence (KL) grade of 1-4.

50. The method of any one of claims 1-49, wherein the subject benefits from treatment of the inflammatory joint disease for at least half a year from the injection of the first dose.

51. The method of any one of claims 1-50, wherein the composition administered in the first and the second dose result in no drug related serious adverse events.

52. The method of any one of claims 1-51, wherein the subject has minimal antibody production to IL-10 following injection of the composition for 360 days.

53. The method of any one of claims 1-52, wherein the subject has no anti-IL-10 antibodies within 360 days following injection of the composition.

54. Use of a composition comprising an IL-10^{F129S} expression construct for the treatment of an inflammatory joint disease, wherein the composition is for administration in a first dose comprising 150 μg to 450 μg of the IL-10^{F129S} expression construct, and wherein the composition is for administration in a second dose comprising 150 μg to 450 μg of the IL-10^{F129S} expression construct 150-360 days after the first dose.

55. A kit comprising a first dose and a second dose of an IL-10^{F129S} expression construct, wherein the first dose comprises 150 μg to 450 μg of the expression construct, and wherein the second dose comprises 150 μg to 450 μg of the expression construct; and
wherein the first and/or the second dose of the composition is formulated in a sterile solution of phosphate-buffered saline (pH 7.4) with about 3% w/v sucrose and about 2.5 mg/mL D-mannose.

56. The kit of claim 55, wherein the first dose comprises 450 μg of the expression construct and the second dose comprises 450 μg of the expression construct.
